(19) United States
(12) Patent Application Publication
(10) Pub. No.: US 2016/0038479 A1
(43) Pub. Date: Feb. 11, 2016

(54) Compositions with Thixotropy and Enhanced Dissolution Reproducibility and Stability

(71) Applicant: DURECT CORPORATION, Cupertino, CA (US)

(72) Inventors: Michael S. Zamloot, Austin, TX (US); Roger Fu, Saratoga, CA (US); Su Il Yum, Los Altos, CA (US); Wendy Chao, San Jose, CA (US); Huey-Ching Su, San Jose, CA (US)

(21) Appl. No.: 14/776,608

(22) PCT Filed: Mar. 14, 2014
(86) PCT No.: PCT/US14/29617

§ 371 Date: Sep. 14, 2015

Related U.S. Application Data
(60) Provisional application No. 61/801,270, filed on Mar. 15, 2013.

(51) Int. Cl. A61K 31/485; A61K 9/48

(52) U.S. Cl. CPC A61K 31/485; A61K 9/4858; A61K 9/485; A61K 9/4866; A61K 9/48; A61K 9/4816

(57) ABSTRACT

The present disclosure provides extended release compositions including, formulations that comprise such compositions, which exhibit desirable dissolution of an active agent while maintaining its physical stability in a dosage form including, for example, providing reduced sample variability, e.g., in the form of reduced inter-capsule variability and/or a reduction in storage-time dependent change in mean release of the active agent from the composition. Related methods of making and administering the disclosed compositions and formulations are also provided.

### CROSS-REFERENCE TO RELATED APPLICATIONS

This application claims the benefit of U.S. Provisional Patent Application No. 61/801,270, filed Mar. 15, 2013.

### INTRODUCTION

Extended release pharmaceutical compositions, including extended release oxycodone compositions, may include various pharmaceutically inactive components which contribute to the desired pharmacokinetic parameters of the active agent in the composition. Such compositions may also include pharmaceutically inactive components which contribute to one or more abuse-deterrent characteristics of the composition. In some such cases, extended release pharmaceutical compositions may be provided which are viscoelastic in nature with a combination of hydrophilic and hydrophobic components. In addition to solubility of the active agent in the composition, the release of the active agent may be controlled, at least in part, by balancing the viscoelastic, hydrophilic and/or hydrophobic nature of the composition. However, in some cases, the viscoelastic, hydrophilic, and/or hydrophobic nature of the composition may also contribute to undesirable sample variability during dissolution of the active agent from the composition. This undesirable sample variability may be evidenced by inter-capsule variability at a particular time point and/or as a storage-time dependent change in mean release of the active agent from the composition (aging). The present disclosure addresses these issues and provides related advantages.

### SUMMARY

The present disclosure provides compositions (e.g., extended release compositions) which exhibit desirable dissolution of an active agent while maintaining its physical stability in a dosage form including, for example, reduced sample variability such as in the form of reduced inter-capsule variability and/or a reduction in storage-time dependent change in mean release of the active agent from the composition (aging). Related methods of making and administering the disclosed compositions and formulations are also provided.

The present disclosure provides a composition comprising: a pharmacologically active agent; about 15% by weight to about 45% (e.g., about 18% to about 27% w/w) by weight, based on total weight of the composition, of a solvent; and about 1% by weight to about 20% (e.g., about 14% to about 19%) by weight, based on total weight of the composition, of a rheology modifier.

In some embodiments of each or any of the above or below mentioned embodiments, the solvent is a hydrophilic solvent.

In some embodiments of each or any of the above or below mentioned embodiments, the composition is within a hydroxypropylmethylcellulose (HPMC) capsule.

In some embodiments of each or any of the above or below mentioned embodiments, the solvent is triacetin, and the rheology modifier is isopropyl myristate (IPM).

In some embodiments of each or any of the above or below mentioned embodiments, the solvent is ethyl lactate, and the rheology modifier is isopropyl myristate (IPM).

In some embodiments of each or any of the above or below mentioned embodiments, the composition comprises a mineral particle.

In some embodiments of each or any of the above or below mentioned embodiments, the mineral particle comprises silicon dioxide, carnauba wax, or cetyl alcohol.

In some embodiments of each or any of the above or below mentioned embodiments, the pharmacologically active agent is selected from opioid, stimulant, and depressant.

In some embodiments of each or any of the above or below mentioned embodiments, the pharmacologically active agent is an opioid.

In some embodiments of each or any of the above or below mentioned embodiments, the pharmacologically active agent is a mu opioid agonist.

In some embodiments of each or any of the above or below mentioned embodiments, the pharmacologically active agent is selected from oxycodone, oxymorphone, hydrocodone, and hydromorphone, either in the free base form or a pharmaceutically acceptable salt form thereof.

In some embodiments of each or any of the above or below mentioned embodiments, the pharmacologically active agent is oxycodone.

In some embodiments of each or any of the above or below mentioned embodiments, the composition does not comprise more than 5% water by weight, based on total weight of the composition.

In some embodiments of each or any of the above or below mentioned embodiments, the composition comprises water at from about 1.0 to about 2.5% by weight, based on total weight of the composition.

The present disclosure provides a composition comprising: a pharmacologically active agent; a solvent; about 1% by weight to about 20% by weight, based on total weight of the composition, of a rheology modifier.

In some embodiments of each or any of the above or below mentioned embodiments, the solvent is a hydrophilic solvent.

In some embodiments of each or any of the above or below mentioned embodiments, the composition is within a hydroxypropylmethylcellulose (HPMC) capsule.

In some embodiments of each or any of the above or below mentioned embodiments, the solvent is triacetin, and the rheology modifier is isopropyl myristate (IPM).

In some embodiments of each or any of the above or below mentioned embodiments, the solvent is ethyl lactate, and the rheology modifier is isopropyl myristate (IPM).

In some embodiments of each or any of the above or below mentioned embodiments, the composition comprises a mineral particle.

In some embodiments of each or any of the above or below mentioned embodiments, the mineral particle comprises silicon dioxide.

In some embodiments of each or any of the above or below mentioned embodiments, the composition comprises a viscosity enhancing agent.

In some embodiments of each or any of the above or below mentioned embodiments, the viscosity enhancing agent comprises silicon dioxide, carnauba wax, or cetyl alcohol.

In some embodiments of each or any of the above or below mentioned embodiments, the pharmacologically active agent is selected from opioid, stimulant, and depressant.

In some embodiments of each or any of the above or below mentioned embodiments, the pharmacologically active agent is an opioid.

In some embodiments of each or any of the above or below mentioned embodiments, the pharmacologically active agent is a mu opioid agonist.

In some embodiments of each or any of the above or below mentioned embodiments, the pharmacologically active agent is selected from oxycodone, oxymorphone, hydrocodone, and hydromorphone, either in the free base form or a pharmaceutically acceptable salt form thereof.

In some embodiments of each or any of the above or below mentioned embodiments, the pharmacologically active agent is oxycodone.

The present disclosure provides a method of orally administering a composition, comprising: reducing a time-dependent change in an in vitro release profile of a composition by formulating the composition to include, in addition to a pharmacologically active agent, about 15% by weight to about 45% by weight, based on total weight of the composition, of a solvent, about 1% by weight to about 20% by weight, based on total weight of the composition, of a rheology modifier, and orally administering the composition.

In some embodiments of each or any of the above or below mentioned embodiments, the solvent is a hydrophilic solvent.

In some embodiments of each or any of the above or below mentioned embodiments, the composition is within a hydroxypropylmethylcellulose (HPMC) capsule.

In some embodiments of each or any of the above or below mentioned embodiments, the solvent is triacetin, and the rheology modifier is isopropyl myristate (IPM).

In some embodiments of each or any of the above or below mentioned embodiments, the solvent is ethyl lactate, and the rheology modifier is isopropyl myristate (IPM).

In some embodiments of each or any of the above or below mentioned embodiments, the composition comprises a mineral particle.

In some embodiments of each or any of the above or below mentioned embodiments, the mineral particle comprises silicon dioxide.

In some embodiments of each or any of the above or below mentioned embodiments, the composition comprises a viscosity enhancing agent.

In some embodiments of each or any of the above or below mentioned embodiments, the viscosity enhancing agent comprises silicon dioxide, carnauba wax, or cetyl alcohol.

In some embodiments of each or any of the above or below mentioned embodiments, the pharmacologically active agent is selected from opioid, stimulant, and depressant.

In some embodiments of each or any of the above or below mentioned embodiments, the pharmacologically active agent is an opioid.

In some embodiments of each or any of the above or below mentioned embodiments, the pharmacologically active agent is a mu opioid agonist.

In some embodiments of each or any of the above or below mentioned embodiments, the pharmacologically active agent is selected from oxycodone, oxymorphone, hydrocodone, and hydromorphone, either in the free base form or a pharmaceutically acceptable salt form thereof.

In some embodiments of each or any of the above or below mentioned embodiments, the pharmacologically active agent is oxycodone.

The present disclosure also provides a method of orally administering a composition, comprising: reducing a time-dependent change in an in vitro release profile of a composition by formulating the composition to include, in addition to a pharmacologically active agent, a solvent; about 1% by weight to about 20% by weight, based on total weight of the composition, of a rheology modifier, and orally administering the composition.

In some embodiments of each or any of the above or below mentioned embodiments, the solvent is a hydrophilic solvent.

In some embodiments of each or any of the above or below mentioned embodiments, the composition is within a hydroxypropylmethylcellulose (HPMC) capsule.

In some embodiments of each or any of the above or below mentioned embodiments, the solvent is triacetin, and the rheology modifier is isopropyl myristate (IPM).

In some embodiments of each or any of the above or below mentioned embodiments, the solvent is ethyl lactate, and the rheology modifier is isopropyl myristate (IPM).

In some embodiments of each or any of the above or below mentioned embodiments, the composition comprises a mineral particle.

In some embodiments of each or any of the above or below mentioned embodiments, the mineral particle comprises silicon dioxide, carnauba wax, or cetyl alcohol.

In some embodiments of each or any of the above or below mentioned embodiments, the pharmacologically active agent is selected from opioid, stimulant, and depressant.

In some embodiments of each or any of the above or below mentioned embodiments, the pharmacologically active agent is an opioid.

In some embodiments of each or any of the above or below mentioned embodiments, the pharmacologically active agent is a mu opioid agonist.

In some embodiments of each or any of the above or below mentioned embodiments, the pharmacologically active agent is selected from oxycodone, oxymorphone, hydrocodone, and hydromorphone, either in the free base form or a pharmaceutically acceptable salt form thereof.

In some embodiments of each or any of the above or below mentioned embodiments, the pharmacologically active agent is oxycodone.

The present disclosure also provides a composition comprising: a pharmacologically active agent; a solvent; a network former; and a mineral particle, wherein the mineral particle is present in the composition in an amount from about 1.9% by weight to about 3.0% by weight relative to the total weight of the composition.

In some embodiments of each or any of the above or below mentioned embodiments, the mineral particle comprises silicon dioxide, carnauba wax, or cetyl alcohol.

In some embodiments of each or any of the above or below mentioned embodiments, the pharmacologically active agent is selected from opioid, stimulant, and depressant.

In some embodiments of each or any of the above or below mentioned embodiments, the pharmacologically active agent is an opioid.

In some embodiments of each or any of the above or below mentioned embodiments, the pharmacologically active agent is a mu opioid agonist.

In some embodiments of each or any of the above or below mentioned embodiments, the pharmacologically active agent is selected from oxycodone, oxymorphone, hydrocodone, and hydromorphone, either in the free base form or a pharmaceutically acceptable salt form thereof.

In some embodiments of each or any of the above or below mentioned embodiments, the pharmacologically active agent is oxycodone.

In some embodiments of each or any of the above or below mentioned embodiments, the solvent comprises triacetin.

In some embodiments of each or any of the above or below mentioned embodiments, the solvent comprises ethyl lactate.

In some embodiments of each or any of the above or below mentioned embodiments, the composition comprises about 15% by weight to about 45% by weight of the solvent relative to the total weight of the composition.

In some embodiments of each or any of the above or below mentioned embodiments, the composition further comprises a rheology modifier.

In some embodiments of each or any of the above or below mentioned embodiments, the rheology modifier is IPM.

In some embodiments of each or any of the above or below mentioned embodiments, the composition comprises about 1% by weight to about 20% by weight of the IPM relative to the total weight of the composition.

In some embodiments of each or any of the above or below mentioned embodiments, the composition comprises: about 35% by weight to about 45% by weight of the HVLCM relative to the total weight of the composition, about 15% by weight to about 45% by weight of the solvent relative to the total weight of the composition, and about 4% by weight to about 5% by weight of the network former relative to the total weight of the composition.

In some embodiments of each or any of the above or below mentioned embodiments, the HVLCM is SAIB, the solvent is triacetin, and the network former is CAB.

In some embodiments of each or any of the above or below mentioned embodiments, the HVLCM is SAIB, the solvent is ethyl lactate, and the network former is CAB.

In some embodiments of each or any of the above or below mentioned embodiments, the composition comprises IPM.

In some embodiments of each or any of the above or below mentioned embodiments, the pharmacologically active agent is present in the composition at about 2% by weight to about 50% by weight relative to the total weight of the composition.

In some embodiments of each or any of the above or below mentioned embodiments, the composition is contained within a capsule.

The present disclosure provides a composition comprising: an opioid; triacetin or ethyl lactate; isopropyl myristate (IPM); and silicon dioxide, wherein the silicon dioxide, is present in the composition in an amount from about 1.9% by weight to about 3.0% by weight relative to the total weight of the composition.

In some embodiments of each or any of the above or below mentioned embodiments, the opioid is selected from oxycodone, oxymorphone, hydrocodone, and hydromorphone, either in the free base form or a pharmaceutically acceptable salt form thereof.

In some embodiments of each or any of the above or below mentioned embodiments, the opioid is oxycodone.

In some embodiments of each or any of the above or below mentioned embodiments, the opioid is present in the composition at about 5% by weight relative to the total weight of the composition.

The present disclosure provides a method for treating pain in a subject, the method comprising: orally administering to the subject a composition comprising an opioid; a solvent; a network former; and silicon dioxide, wherein the silicon dioxide is present in the composition in an amount from about 1.9% by weight to about 3.0% by weight relative to the total weight of the composition, wherein the composition is formulated for oral administration, and one or more symptoms or signs associated with the subject's pain is alleviated.

In some embodiments of each or any of the above or below mentioned embodiments, the opioid is selected from oxycodone, oxymorphone, hydrocodone, and hydromorphone, either in the free base form or a pharmaceutically acceptable salt form thereof.

In some embodiments of each or any of the above or below mentioned embodiments, the opioid is oxycodone.

In some embodiments of each or any of the above or below mentioned embodiments, the solvent comprises triacetin.

In some embodiments of each or any of the above or below mentioned embodiments, the solvent comprises ethyl lactate.

In some embodiments of each or any of the above or below mentioned embodiments, the composition comprises about 15% by weight to about 45% by weight of the solvent relative to the total weight of the composition.

In some embodiments of each or any of the above or below mentioned embodiments, the composition further comprises a rheology modifier.

In some embodiments of each or any of the above or below mentioned embodiments, the rheology modifier is IPM.

In some embodiments of each or any of the above or below mentioned embodiments, the pharmacologically active agent is present in the composition at about 2% by weight to about 50% by weight relative to the total weight of the composition.

In some embodiments of each or any of the above or below mentioned embodiments, the composition is contained within a capsule.

In some embodiments of each or any of the above or below mentioned embodiments, the composition is administered no more than twice in a 24-hour period.

The present disclosure also provides a method for treating pain in a subject, the method comprising: orally administering to the subject a composition comprising an opioid; triacetin or ethyl lactate; isopropyl myristate (IPM); and silicon dioxide, wherein the silicon dioxide, is present in the composition in an amount from about 1.9% by weight to about 3.0% by weight relative to the total weight of the composition, wherein the composition is formulated for oral administration, and one or more symptoms or signs associated with the subject's pain is alleviated.

In some embodiments of each or any of the above or below mentioned embodiments, the opioid is selected from oxycodone, oxymorphone, hydrocodone, and hydromorphone, either in the free base form or a pharmaceutically acceptable salt form thereof.

In some embodiments of each or any of the above or below mentioned embodiments, the opioid is oxycodone.

In some embodiments of each or any of the above or below mentioned embodiments, the opioid is present in the composition at about 5% by weight relative to the total weight of the composition.

In some embodiments of each or any of the above or below mentioned embodiments, the composition is encapsulated for oral administration.

In some embodiments of each or any of the above or below mentioned embodiments, the composition is contained within a capsule.

In some embodiments of each or any of the above or below mentioned embodiments, the composition is administered no more than twice in a 24-hour period.

The present disclosure also provides a method of orally administering a composition, comprising: improving reproducibility of an in vitro release profile of a composition by including about 1.9% by weight to about 3.0% by weight, relative to the total weight of the composition, of mineral particle in the composition, wherein the composition also includes a pharmacologically active agent, and a solvent; and orally administering the composition.

The present disclosure also provides a method of orally administering a composition, comprising: decreasing the variability of an in vitro release profile of a composition by including about 1.9% by weight to about 3.0% by weight, relative to the total weight of the composition, of mineral particle in the composition, wherein the composition also includes a pharmacologically active agent, a solvent; and orally administering the composition.

The present disclosure also provides a method of orally administering an encapsulated composition, comprising: forming a composition comprising: a pharmacologically active agent, a solvent, and a mineral particle, wherein the mineral particle is present in the composition in an amount from about 1.9% by weight to about 3.0% by weight relative to the total weight of the composition; improving an in vitro release profile of the composition by encapsulating the composition within a capsule comprising hydroxypropylmethylcellulose to form an encapsulated composition; and orally administering the encapsulated composition.

The present disclosure also provides a method of orally administering an encapsulated composition, comprising: forming a composition comprising: a pharmacologically active agent, a solvent, and a mineral particle, wherein the mineral particle is present in the composition in an amount from about 1.9% by weight to about 3.0% by weight relative to the total weight of the composition; reducing exposure of the composition to water by encapsulating the composition within a capsule to form an encapsulated composition; and orally administering the encapsulated composition.

The present disclosure also provides a composition comprising: a pharmacologically active agent (e.g., about 2% by weight to about 50% by weight, relative to the total weight of the composition, of an opioid, such as an opioid selected from oxycodone, oxymorphone, hydrocodone, and hydromorphone, either in the free base form or a pharmaceutically acceptable salt form thereof); triacetin; about 4% by weight to about 5% by weight of cellulose acetate butyrate (CAB) relative to the total weight of the composition; sucrose acetate isobutyrate (SAIB); isopropyl myristate (IPM); and a mineral particle (e.g., silicon dioxide), wherein the mineral particle is present in the composition in an amount from about 1.9% by weight to about 3.0% by weight (e.g., about 2.4% by weight to about 3.0% by weight), relative to the total weight of the composition, wherein the composition is contained within a capsule (e.g., a hydroxypropylmethylcellulose capsule). In some embodiments, the composition does not comprise more than 5% water by weight, based on total weight of the composition. For instance, the composition may comprise water at from about 1.0 to about 2.5% by weight, based on total weight of the composition.

### BRIEF DESCRIPTION OF THE DRAWINGS

FIG. 1 shows the mean oxycodone concentration-time profiles for Formulation 1, Formulation 2, and Formulation 3

FIG. 2 is a flow chart providing materials and methods for the preparation of selected hydromorphone HCl compositions.

FIG. 3 provides graphs showing the results of in vitro dissolution experiments for Reference Formulation A (with BHT) (Panel A) and Formulations 7 (Panel B) and 8 (Panel C).

FIG. 4 provides graphs showing the results of in vitro dissolution experiments for Reference Formulation A (with BHT) (Panel A) and Formulations 9 (Panel B) and 10 (Panel C).

FIG. 5 provides graphs showing the effects of IPM (Panel A) and SiO₂ (Panel B) on mean release of oxycodone relative to Reference Formulation A (with BHT).

FIG. 6 is a graph showing the effect of SiO₂ on an oxycodone mean release profile. Results for Formulation A′, and Formulations 11 and 12 are shown.

FIG. 7 provides graphs showing the effect of increased amounts of SiO₂ on inter-capsule variability during dissolution. Results for Formulation A′ (Panel A), and Formulations 11 (Panel B) and 12 (Panel C) are shown.

FIG. 8 is a graph showing the complex viscosity profiles for Formulations A′, 11 and 12. Increasing SiO₂ concentration above about 2% increases complex viscosity which may lead to decreasing reproducible deformation and therefore low inter-capsule variability during dissolution testing.

FIG. 9 is a graph showing mean release of oxycodone from Formulation A′ following storage for 1 month at 25° C. or 40° C.

FIG. 10 provides graphs showing inter-capsule variability during dissolution testing of Formulation A′ following storage for 1 month at 25° C. or 40° C.

FIG. 11 is a graph showing mean release of oxycodone from Formulation 11 following storage for 1 month at 25° C. or 40° C.

FIG. 12 provides graphs showing inter-capsule variability during dissolution testing of Formulation 11 following storage for 1 month at 25° C. or 40° C.

FIG. 13 is a graph showing mean release of oxycodone from Formulation 12 following storage for 1 month at 25° C. or 40° C.

FIG. 14 provides graphs showing inter-capsule variability during dissolution testing of Formulation 12 following storage for 1 month at 25° C. or 40° C.

FIG. 15 provides a flow chart showing exemplary materials and methods utilized in the preparation of hydromorphone hydrochloride formulations according to the present disclosure.

FIG. 16 is a graph showing Day 1 mean oxymorphone concentration-time data after administration of the control article Opana ER 20 mg and the test article Formulation 102 (20 mg).

FIG. 17 is a graph showing Day 1 mean oxymorphone concentration-time data after administration of the control article Opana ER 20 mg and the test article Formulation 102 (20 mg).

FIG. 18 is a graph showing Day 5 Mean oxymorphone concentration-time data after administration of the control article Opana ER 20 mg and the test article Formulation 102 20 mg twice daily.

FIG. 19 is a graph showing Day 5 Mean oxymorphone concentration-time data after administration of the control article Opana ER 20 mg and the test article Formulation 102 20 mg twice daily.

FIG. 20 is a graph showing Day 1 mean 6β-Hydroxyoxymorphone concentration-time data after administration of the control article Opana ER 20 mg and the test article formulation 102 (20 mg).

FIG. 21 is a graph showing Day 1 mean 6β-Hydroxyoxymorphone concentration-time data after administration of the control article Opana ER 20 mg and the test article formulation 102 (20 mg).

FIG. 22 is a graph showing Day 5 mean 6β-Hydroxyoxymorphone concentration-time data after administration of the Control Article Opana ER 20 mg and the test article Formulation 102 20 mg Twice Daily.

FIG. 23 is a graph showing Day 5 mean 6β-Hydroxyoxymorphone concentration-time data after administration of the Control Article Opana ER 20 mg and the test article Formulation 102 20 mg Twice Daily.

FIG. 24 is a graph showing Day 1 mean Oxymorphone-Glucuronide concentration-time data after administration of the control article Opana ER 20 mg and the test article Formulation 102 20 mg.

FIG. 25 is a graph showing Day 1 mean Oxymorphone-Glucuronide concentration-time data after administration of the control article Opana ER 20 mg and the test article Formulation 102 20 mg.

FIG. 26 is a graph showing Day 5 mean Oxymorphone-Glucuronide concentration-time data after administration of the control article Opana ER 20 mg and the test article Formulation 102 20 mg Twice Daily.

FIG. 27 is a graph showing Day 5 mean Oxymorphone-Glucuronide concentration-time data after administration of the control article Opana ER 20 mg and the test article Formulation 102 20 mg Twice Daily.

FIG. 28 is a graph showing Day 1 mean total exposure-time data after administration of the control article Opana ER 20 mg and the test article formulation 102 20 mg.

FIG. 29 is a graph showing Day 1 mean total exposure-time data after administration of the control article Opana ER 20 mg and the test article formulation 102 20 mg.

FIG. 30 is a graph showing Day 5 mean total exposure-time data after administration of the control article Opana ER 20 mg and the Test Article Formulation 102 20 mg Twice Daily.

FIG. 31 is a graph showing Day 5 mean total exposure-time data after administration of the control article Opana ER 20 mg and the Test Article Formulation 102 20 mg Twice Daily.

FIG. 32 is a graph showing initial dissolution results (T0) for select formulations in gelatin and HPMC capsules.

FIG. 33 is a graph showing cumulative % drug release over time for a formulation in hard gelatin capsules with storage conditions of 1 month at 25° C. and 40° C., or 30 months at 25° C.

FIG. 34 is a graph showing cumulative % drug release over time for the formulation of FIG. 33 in HPMC capsules with storage conditions of 1 month at 25° C. and 40° C., or 30 months at 25° C.

FIG. 35 is a graph showing cumulative % drug release over time for a formulation in hard gelatin capsules with storage conditions of 1 month at 25° C. and 40° C., or 30 months at 25° C.

FIG. 36 is a graph showing cumulative % drug release over time for the formulation of FIG. 35 in HPMC capsules with storage conditions of 1 month at 25° C. and 40° C., or 30 months at 25° C.

### DEFINITIONS

As used interchangeably herein, the terms “active agent”, “pharmacologically active agent” and “beneficial agent” refer to any substance intended for use in the diagnosis, cure, mitigation, treatment, or prevention of any disease, disorder, or condition or intended to affect the structure or function of the body, other than food. It can include any beneficial agent or substance that is biologically active or meant to alter animal physiology.

As used herein, the term “formulation” refers to one or more ingredients or compounds. For example, a drug formulation is any drug combined together with any pharmaceutically acceptable excipients, additives, solvents, carriers and other materials.

As used herein, the term “high viscosity liquid carrier material (HVLCM)” refers to a non-polymeric, non-water soluble liquid material having a viscosity of at least 5000 cP at 37° C. that does not crystallize neat at 25° C. and 1 atmosphere.

As used herein, the term “rheology modifier” refers to a substance that possesses both a hydrophobic and a hydrophilic moiety. Rheology modifiers suitable for use in the disclosed compositions and methods generally have a logarithm of octanol-water partition coefficient (“Log P”) of between about −7 and +15, e.g., between −5 and +10, e.g., between −1 and +7.

As used herein, the term “network former” refers to a material or compound that forms a network structure when introduced into a liquid medium (such as a HVLCM).

As used herein, the term “hydrophilic agent” means a compound or material having a natural affinity for aqueous systems. A material may be regarded as a hydrophilic agent for the purposes of this disclosure if the material displays a water sorption between about 10 to 100% (w/w). Hydrophilic agents will have a low Log P value, for example, a Log P of less than +1.

As used herein, the term “hydrophilic solvent” means a solvent meeting the definition of a hydrophilic agent as described above.

The term “solvent”, as used herein, refers to any substance that dissolves another substance (solute).

As used herein, the term “treatment”, “treat” and “treating” pain refers to eliminating, reducing, suppressing or ameliorating, either temporarily or permanently, either partially or completely, a clinical symptom, manifestation or progression of pain. In addition, or alternatively, the terms “treatment”, “treat” and “treating” as used herein with respect to the methods as described refer to inhibiting, delaying, suppressing, reducing, eliminating or ameliorating, either temporarily or permanently, either partially or completely, pain. In some embodiments the treating is effective to reduce a symptom, sign, and/or condition of pain in a subject by at least about 10% (e.g., 15%, 20%, 25%, 30%, 35%, 40%, 45%, 50%, 55%, 60%, 65%, 70%, 75%, 80%, 85%, 90%, 95%, or 100%) including, as compared to a baseline measurement of the symptom, sign, and/or condition made prior to the treatment. In some embodiments, the treating is effective to improve an assessment used to diagnose pain in a subject including, as compared to a baseline assessment made prior to the treatment. Such treating as provided herein need not be absolute to be useful.

The term “pharmaceutically acceptable salt,” as used herein, intends those salts that retain the biological effectiveness and properties of neutral active agents and are not otherwise unacceptable for pharmaceutical use.

As used herein, the term “viscosity enhancing agent” refers to a compound or material that can be added to an extended release composition in order to increase the viscosity of the resulting composition.

As used herein, the term “stabilizer” refers to any substance used to inhibit or reduce degradation (e.g., chemical) of other substances with which the stabilizer is mixed.

As used herein, the term “thixotropy” refers to the property exhibited by a composition of becoming a liquid (e.g., a decrease in viscosity) when a stress is applied to the composition.

The terms “% w/w” and “w %” are used interchangeably herein to refer to percent weight per weight.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a composition” includes a plurality of such compositions and reference to “the capsule” includes reference to one or more capsules and equivalents thereof known to those skilled in the art, and so forth. It is further noted that the claims may be drafted to exclude any element, e.g., any optional element. As such, this statement is intended to serve as antecedent basis for use of such exclusive terminology as “solely,” “only” and the like in connection with the recitation of claim elements, or use of a “negative” limitation.

To the extent the definition or usage of any term herein conflicts with a definition or usage of a term in an application or reference incorporated by reference herein, the instant application shall control.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. This is intended to provide support for all such combinations.

### DETAILED DESCRIPTION

As discussed previously herein, the viscoelastic, hydrophilic and/or hydrophobic nature of a pharmaceutical composition may contribute to undesirable sample variability during dissolution of the active agent from the composition. This undesirable sample variability may be evidenced by inter-capsule variability at a particular time point and/or as a storage-time dependent change in mean release of the active agent from the composition.

The present disclosure provides extended release compositions, including formulations that comprise such compositions, which exhibit desirable dissolution of an active agent while maintaining its physical stability in a dosage form including, for example, providing reduced sample variability such as in the form of reduced in vitro inter-capsule variability and/or a reduction in storage-time dependent change in mean in vitro release of the active agent from the composition.

Suitable in vitro dissolution test conditions for determining a time-dependent change in an in vitro release profile of a composition or inter-capsule variability of a composition, e.g., an oxycodone or hydrocodone containing composition are as follows: a USP Apparatus 2 dissolution tester modified to include a 20 mesh screen hanging basket to hold the test article is utilized with dissolution medium containing 1000 ml 0.1 N HCl with 0.5% (w/w) SDS. The dissolution medium is maintained at 37° C. with stirring with 100 rpm paddle speed over the course of a 24 hour dissolution test. Standard sampling time points of 0.5, 2, 3, 6, 12 and 24 hours are utilized. A 1 mL sample is taken at each time point and assayed using reverse-phase HPLC at 240 nm wavelength with a mobile phase including 0.35% (w/v) SDS/0.7% (v/v) acetic acid/44% (v/v) acetonitrile in water. Where the dissolution test is used to determining a time-dependent change in an in vitro release profile of a composition, the composition may be stored for a suitable period of time prior to testing, e.g., the composition may be stored at 25° C./60% relative humidity (RH) for from 1 to 6 months or at 40° C./75% RH for from 1 to 6 months. A suitable number of capsules per composition tested may be, e.g., 12 capsules.

Notably, it is demonstrated herein that the addition of Cab-o-Sil® (i.e., silicon dioxide) to an extended release matrix confers thixotropy and improves dissolution stability. Related methods of making and administering the disclosed compositions including, formulations that comprise such compositions, are also provided. The compositions and formulations of the present disclosure generally include a pharmacologically active agent, a solvent, and a mineral particle. In some embodiments, the compositions and formulations also include one or more of a rheology modifier, a network former, a hydrophilic agent, a viscosity enhancing agent and a stabilizing agent.

### Pharmacologically Active Agent

The pharmacologically active agents that may be included in the compositions of the present disclosure may include any type of biologically active compound or composition of matter which, when administered to an organism (human or animal subject) induces a desired pharmacologic and/or physiologic effect by local and/or systemic action.

Examples of such biologically active compounds or compositions of matter useful in the disclosed compositions include, but are not limited to, opioids, CNS depressants and stimulants.

Opioids are a class of potent narcotics that includes, for example, morphine, codeine, oxycodone and fentanyl and related drugs. Morphine is often used to alleviate severe pain. Codeine is used for milder pain. Other examples of opioids that can be prescribed to alleviate pain include oxycodone (e.g. OxyContin®-an oral, controlled release form of the drug); propoxyphene (e.g. Darvon™); hydrocodone (e.g. Vicodin™); hydromorphone (e.g. Dilaudid™); and meperidine (e.g. Demerol™).

In addition to relieving pain, opioids can also produce a sensation of euphoria, and when taken in large doses, can cause severe respiratory depression which can be fatal.

CNS depressants slow down normal brain function by increasing GABA activity, thereby producing a drowsy or calming effect. In higher doses, some CNS depressants can become general anesthetics, and in very high doses may cause respiratory failure and death. CNS depressants are frequently abused, and often the abuse of CNS depressants occurs in conjunction with the abuse of another substance or drug, such as alcohol or cocaine. Many deaths occur yearly through such drug abuse. CNS depressants can be divided into two groups, based on their chemistry and pharmacology: (1) Barbiturates, such as mephobarbital (e.g. Mebaral™) and pentobarbital sodium (e.g. Nembutal™), which are used to treat anxiety, tension, and sleep disorders. (2) Benzodiazepines, such as diazepam (e.g. Valium™), chlordiazepoxide HCl (e.g. Librium™), and alprazolam (e.g. Xanax™), which can be prescribed to treat anxiety, acute stress reactions, and panic attacks. Benzodiazepines that have a more sedating effect, such as triazolam (e.g. Halcion™) and estazolam (e.g. ProSom™) can be prescribed for short-term treatment of sleep disorders.

Stimulants are a class of drugs that enhance brain activity—they cause an increase in alertness, attention, and energy that is accompanied by increases in blood pressure, heart rate, and respiration. Stimulants are frequently prescribed for treating narcolepsy, attention-deficit hyperactivity disorder (ADHD), and depression. Stimulants may also be used for short-term treatment of obesity, and for patients with asthma. Stimulants such as dextroamphetamine (Dexedrine™) and methylphenidate (Ritalin™) have chemical structures that are similar to key brain neurotransmitters called monoamines, which include norepinephrine and dopamine. Stimulants increase the levels of these chemicals in the brain and body. This, in turn, increases blood pressure and heart rate, constricts blood vessels, increases blood glucose, and opens up the pathways of the respiratory system. In addition, the increase in dopamine is associated with a sense of euphoria that can accompany the use of these drugs.

Taking high doses of a stimulant can result in an irregular heartbeat, dangerously high body temperatures, and/or the potential for cardiovascular failure or lethal seizures. Taking high doses of some stimulants repeatedly over a short period of time can lead to hostility or feelings of paranoia in some individuals.

One class of biologically active compounds that may be included in the compositions of the present disclosure is the opioids class, which includes alfentanil, allylprodine, alphaprodine, anileridine, apomorphine, apocodeine, benzylmorphine, bezitramide, buprenorphine, butorphanol, clonitazene, codeine, cyclazocine, cyclorphen, cyprenorphine, desomorphine, dextromoramide, dextromethorphan, dezocine, diampromide, dihydrocodeine, dihydromorphine, dimenoxadol, dimepheptanol, dimethylthiambutene, dioxyaphetyl butyrate, dipipanone, eptazocine, ethoheptazine, ethylmethylthiambutene, ethylmorphine, etonitazene, fentanyl, heroin, hydrocodone, hydroxymethylmorphinan, hydromorphone, hydroxypethidine, isomethadone, ketobemidone, levallorphan, levorphanol, levophenacylmorphan, levomethorphan, lofentanil, meperidine, meptazinol, metazocine, methadone, methylmorphine, metopon, morphine, myrophine, nalbuphine, narceine, nicomorphine, norlevorphanol, normethadone, nalorphine, normorphine, norpipanone, ohmefentanyl, opium, oxycodone, oxymorphone, papaveretum, pentazocine, phenadoxone, phenomorphan, phenazocine, phenoperidine, pholcodine, piminodine, piritramide, propheptazine, promedol, profadol, properidine, propiram, propoxyphene, remifentanyl, sufentanyl, tramadol, tilidine, naltrexone, naloxone, nalmefene, methylnaltrexone, naloxone methiodide, nalorphine, naloxonazine, nalide, nalmexone, nalbuphine, nalorphine dinicotinate, naltrindole (NTI), naltrindole isothiocyanate (NTII), naltriben (NTB), nor-binaltorphimine (nor-BNI), tapentadol, beta-funaltrexamine (b-FNA), 7-Benzylidenenaltrexone (BNTX), cyprodime, N,N-diallyl-Tyr-Aib-Aib-Phe-Leu (ICI-174,864), 3-[1-(3-hydroxy-3-phenylpropyl)-3,4-dimethylpiperidin-4-yl]phenol (LY117413), [(−)-(1R,5R,9R)-5,9-diethyl-2-(3-furylmethyl)-2′-hydroxy-6,7-benzomorphan] (MR2266), etorphine, [D-Ala², NMe-Phe⁴, Gly-ol⁵]-enkephalin (DAMGO), CTOP (CAS No:103429-31-8), diprenorphine, naloxone benzoylhydrazone, bremazocine, ethylketocyclazocine, (U50,488), (U69,593), spiradoline, [D-Pen^{2,5}]Enkephalin (DPDPE), [D-Ala2,Glu4] deltorphin, [D-Ser², Leu⁵, Thr⁶]-enkephalin (DSLET), Met-enkephalin, Leu-enkephalin, β-endorphin, dynorphin A, dynorphin B, a-neoendorphin, or an opioid having the same pentacyclic nucleus as nalmefene, naltrexone, buprenorphine, levorphanol, meptazinol, pentazocine, dezocine, or their pharmacologically effective esters or salts.

In some embodiments, opioids for use in the compositions of the present disclosure are selected from morphine, hydrocodone, oxycodone, codeine, fentanyl (and its relatives), hydromorphone, meperidine, methadone, oxymorphone, propoxyphene or tramadol, or mixtures thereof. In some embodiments, opioids for use in the compositions of the present disclosure are selected from oxycodone, oxymorphone, hydrocodone and hydromorphone. In some embodiments, the opioids for use in the compositions of the present disclosure may be micronized. In some embodiments, the opioid may be provided in either in the free base form or a pharmaceutically acceptable salt form. With respect to the opioid oxycodone, it may be beneficial to provide formulations that have a reduced level of peroxide degradation products such as alpha beta unsaturated ketones (ABUK). In such cases, the compositions of the present disclosure can be subjected to peroxide contaminant reduction and/or removal techniques in accordance with known methods.

Other pharmacologically active compounds or compositions of matter useful in the disclosed compositions include prochlorperazine edisylate, ferrous sulfate, aminocaproic acid, potassium chloride, mecamylamine, procainamide, amphetamine (all forms including dexamphetamine, dextroamphetamine, d-S-amphetamine, and levo amphetamine), benzphetamine, isoproternol, methamphetamine, dexmethamphetamine, phenmetrazine, bethanechol, metacholine, pilocarpine, atropine, methascopolamine, isopropamide, tridihexethyl, phenformin, methylphenidate (all forms including dexmethylphenidate, d-threo methylphenidate, and dl-threo methylphenidate), oxprenolol, metroprolol, cimetidine, diphenidol, meclizine, prochlorperazine, phenoxybenzamine, thiethylp erazine, anisindone, diphenadione erythrityl, digoxin, isofurophate, reserpine, acetazolamide, methazolamide, bendroflumethiazide, chlorpropamide, tolazamide, chlormadinone, phenaglycodol, allopurinol, aluminum aspirin, methotrexate, acetyl sulfisoxazole, erythromycin, progestins, estrogenic progrestational, corticosteroids, hydrocortisone, hydrocorticosterone acetate, cortisone acetate, triamcinolone, methyltesterone, 17 beta-estradiol, ethinyl estradiol, ethinyl estradiol 3-methyl ether, prednisolone, 17-hydroxypro gesterone acetate, 19-nor-progesterone, norgestrel, orethindone, norethiderone, progesterone, norgestrone, norethynodrel, aspirin, indomethacin, naproxen, fenoprofen, sulindac, diclofenac, indoprofen, nitroglycerin, propranolol, metroprolol, sodium valproate, valproic acid, taxanes such as paclitaxel, camptothecins such as 9-aminocamptothecin, oxprenolol, timolol, atenolol, alprenolol, cimetidine, clonidine, imipramine, levodopa, chloropropmazine, resperine, methyldopa, dihydroxyphenylalanine, pivaloyloxyethyl ester of α-methyldopa hydrochloride, theophylline, calcium gluconate ferrous lactate, ketoprofen, ibuprofen, cephalexin, haloperiodol, zomepirac, vincamine, diazepam, phenoxybenzamine, β-blocking agents, calcium-channel blocking drugs such as nifedipine, diltiazen, verapamil, lisinopril, captopril, ramipril, fosimopril, benazepril, libenzapril, cilazapril cilazaprilat, perindopril, zofenopril, enalapril, indalapril, qumapril, and the like.

The active agent can be present in the compositions of the present disclosure in a neutral form, as a free base form, or in the form of a pharmaceutically acceptable salt. Pharmaceutically acceptable salts include salts of acidic or basic groups, which groups may be present in the active agents. Those active agents that are basic in nature are capable of forming a wide variety of salts with various inorganic and organic acids. Pharmaceutically acceptable acid addition salts of basic active agents suitable for use herein are those that form non-toxic acid addition salts, i.e., salts comprising pharmacologically acceptable anions, such as the hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Active agents that include an amino moiety may form pharmaceutically acceptable salts with various amino acids, in addition to the acids mentioned above. Suitable base salts can be formed from bases which form non-toxic salts, for example, aluminium, calcium, lithium, magnesium, potassium, sodium, zinc and diethanolamine salts. See, e.g., Berge et al. (1977) J. Pharm. Sci. 66:1-19, the disclosure of which is incorporated by reference herein.

In the compositions of the present disclosure, the pharmacologically active agent will be dissolved (fully or partially) in one or more components of the composition or dispersed within one or more components of the composition. The phrase “dissolved or dispersed” is intended to encompass all means of establishing a presence of the active agent in the subject compositions and includes dissolution, dispersion, partial dissolution and dispersion, and/or suspension and the like. In addition, in certain embodiments of the present disclosure wherein the active agent is in a solid particulate form suspended within one or more other components of the composition, the active agent particulate may be pre-treated with a micronization process such as those described in U.S. Application Publication No. 2009/0215808, the disclosure of which is incorporated by reference herein, to provide a particle population having a substantially homogeneous particle size the bulk of which fall within the micron (μm) range.

The pharmacologically active agent, which can include one or more suitable active agent, may be present in the disclosed compositions in an amount of from about 50 to about 0.1 percent by weight relative to the total weight of the composition (wt %), e.g., in an amount of from about 40 to about 0.1 wt %, in an amount of from about 30 to about 0.1 wt %, in an amount of from about 20 to about 0.1 wt %, in an amount of from about 10 to about 0.1 wt %, in an amount of from about 9 to about 0.1 wt %, in an amount of from about 8 to about 0.1 wt %, in an amount of from about 7 to about 0.1 wt %, in an amount of from about 6 to about 0.1 wt %, in an amount of from about 5 to about 0.1 wt %, in an amount of from about 4 to about 0.1 wt %, in an amount of from about 3 to about 0.1 wt %, in an amount of from about 2 to about 0.1 wt %, or in an amount of from about 1 to about 0.1 wt %, depending upon the identity of the active agent, the desired dose required for the dosage form, and the intended use thereof.

In some embodiments, the pharmacologically active agent may be present in the disclosed compositions in an amount from about 0.1 to about 5 w %, in an amount from about 5 to about 10 w %, in an amount from about 10 to about 20 w %, in an amount from about 20 to about 30 w %, in an amount from about 30 to about 40 w %, or in an amount from about 40 to about 50 w %, depending upon the identity of the active agent, the desired dose required for the dosage form, and the intended use thereof.

In some embodiments, the active agent is present in the composition in an amount of about 1 to about 10 wt %, and can thus be loaded into a suitable dosage form to provide single dosages ranging from about 0.01 mg to about 1000 mg, or from about 0.1 mg to about 500 mg, or from about 2 mg to about 250 mg, or from about 2 mg to about 250 mg, or from about 2 mg to about 150 mg, or from about 5 mg to about 100 mg, or from about 5 mg to about 80 mg. For example, in some embodiments, the active agent is present in the composition in an amount of from about 2 wt % to about 9 wt %, from about 3 wt % to about 8 wt %, from about 4 wt % to about 7 wt %, or from about 5 wt % to about 6 wt %. In some embodiments, the the active agent is present in the composition in an amount of about 1 wt %, about 2 wt %, about 3 wt %, about 4 wt %, about 5 wt %, about 6 wt %, about 7 wt %, about 8 wt %, about 9 wt %, or about 10 wt %.

For some embodiments that include an opioid active agent, exemplary single dosages include, but are not limited to, about 1, about 2, about 3, about 4, about 5, about 10, about 15, about 20, about 25, about 30, about 35, about 40, about 45, about 50, about 55, about 60, about 65, about 70, about 75, about 80, about 85, about 90, about 95, about 100, about 110, about 120, about 130, about 140, about 150 and about 160 mg.

In other embodiments that include a CNS depressant or CNS stimulant, exemplary single dosages include, but are not limited to, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, and about 100 mg.

In some embodiments, where the active agent includes oxycodone free base, the active agent is present in the composition in an amount of from about 50 to about 0.1 percent by weight relative to the total weight of the composition (wt %), e.g., in an amount of from about 40 to about 0.1 wt %, in an amount of from about 30 to about 0.1 wt %, in an amount of from about 20 to about 0.1 wt %, in an amount of from about 10 to about 0.1 wt %, in an amount of from about 9 to about 0.1 wt %, in an amount of from about 8 to about 0.1 wt %, in an amount of from about 7 to about 0.1 wt %, in an amount of from about 6 to about 0.1 wt %, in an amount of from about 5 to about 0.1 wt %, in an amount of from about 4 to about 0.1 wt %, in an amount of from about 3 to about 0.1 wt %, in an amount of from about 2 to about 0.1 wt %, or in an amount of from about 1 to about 0.1 wt %.

In some embodiments, where the active agent includes oxycodone free base, the active agent may be present in the disclosed compositions in an amount from about 0.1 to about 5 w %, in an amount from about 5 to about 10 w %, in an amount from about 10 to about 20 w %, in an amount from about 20 to about 30 w %, in an amount from about 30 to about 40 w %, or in an amount from about 40 to about 50 w %.

In some embodiments, where the active agent comprises oxycodone free base, the active agent is present in the composition in an amount of about 1 to about 10 wt %, and can thus be loaded into a suitable dosage form to provide single dosages ranging from about 0.01 mg to about 1000 mg, or from about 0.1 mg to about 500 mg, or from about 2 mg to about 250 mg, or from about 2 mg to about 250 mg, or from about 2 mg to about 150 mg, or from about 5 mg to about 100 mg, or from about 5 mg to about 80 mg. For example, in some embodiments, where the active agent comprises oxycodone free base, the active agent is present in the composition in an amount of from about 2 wt % to about 9 wt %, from about 3 wt % to about 8 wt %, from about 4 wt % to about 7 wt %, or from about 5 wt % to about 6 wt %. In some embodiments, where the active agent comprises oxycodone free base, the the active agent is present in the composition in an amount of about 1 wt %, about 2 wt %, about 3 wt %, about 4 wt %, about 5 wt %, about 6 wt %, about 7 wt %, about 8 wt %, about 9 wt %, or about 10 wt %.

For some embodiments, where the active agent comprises oxycodone free base, exemplary single dosages include, but are not limited to, about 1, about 2, about 3, about 4, about 5, about 10, about 15, about 20, about 25, about 30, about 35, about 40, about 45, about 50, about 55, about 60, about 65, about 70, about 75, about 80, about 85, about 90, about 95, about 100, about 110, about 120, about 130, about 140, about 150, and about 160 mg.

In some embodiments, where the active agent is oxycodone free base, the active agent is present in the composition in an amount of about 1 to about 10 wt %, and can thus be loaded into a suitable dosage form to provide single dosages ranging from about 0.01 mg to 1000 mg, or from about 0.1 mg to 500 mg, or from about 2 mg to 250 mg, or from about 2 mg to 250 mg, or from about 2 mg to 150 mg, or from about 5 mg to 100 mg, or from about 5 mg to 80 mg. For example, in some embodiments, the oxycodone free base is present in the composition in an amount of from about 2 wt % to about 9 wt %, from about 3 wt % to about 8 wt %, from about 4 wt % to about 7 wt %, or from about 5 wt % to about 6 wt %.

In some embodiments, the oxycodone free base is present in the composition in an amount of about 1 wt %, about 2 wt %, about 3 wt %, about 4 wt %, about 5 wt %, about 6 wt %, about 7 wt %, about 8 wt %, about 9 wt %, or about 10 wt %.

The precise amount of active agent desired can be determined by routine methods well known to pharmacological arts, and will depend on the type of agent, and the pharmacokinetics and pharmacodynamics of that agent.

### High Viscosity Liquid Carrier Material (HVLCM)

An HVLCM is a non-polymeric, non-water soluble liquid material having a viscosity of at least 5000 cP at 37° C. that does not crystallize neat at 25° C. and 1 atmosphere. The term “non-water soluble” refers to a material that is soluble in water to a degree of less than one percent by weight at 25° C. and 1 atmosphere. The term “non-polymeric” refers to esters or mixed esters having essentially no repeating units in the acid moiety of the ester, as well as esters or mixed esters having acid moieties wherein functional units in the acid moiety are repeated a small number of times (i.e., oligomers). Generally, materials having more than five identical and adjacent repeating units or mers in the acid moiety of the ester are excluded by the term “non-polymeric” as used herein, but materials containing dimers, trimers, tetramers, or pentamers are included within the scope of this term. When the ester is formed from hydroxy-containing carboxylic acid moieties that can further esterify, such as lactic acid or glycolic acid, the number of repeat units is calculated based upon the number of lactide or glycolide moieties, rather than upon the number of lactic acid or glycolic acid moieties, where a lactide repeat unit contains two lactic acid moieties esterified by their respective hydroxy and carboxy moieties, and where a glycolide repeat unit contains two glycolic acid moieties esterified by their respective hydroxy and carboxy moieties. Esters having 1 to about 20 etherified polyols in the alcohol moiety thereof, or 1 to about 10 glycerol moieties in the alcohol moiety thereof, are considered non-polymeric as that term is used herein. HVLCMs may be carbohydrate-based, and may include one or more cyclic carbohydrates chemically combined with one or more carboxylic acids. HVLCMs also include non-polymeric esters or mixed esters of one or more carboxylic acids, having a viscosity of at least 5,000 cP at 37° C., that do not crystallize neat at 25° C. and 1 atmosphere, wherein when the ester contains an alcohol moiety (e.g., glycerol). The ester may, for example comprise from about 2 to about 20 hydroxy acid moieties. Various HVLCMs, which may be used be included in disclosed compositions are described in U.S. Pat. Nos. 5,747,058; 5,968,542; and 6,413,536; the disclosures of each of which are incorporated by reference herein. The presently disclosed compositions may employ any HVLCM described in these patents but is not limited to any specifically described materials.

The HVLCM may be present in the composition at from about 35% by weight to about 45% by weight, based on total weight of the composition. For example, the HVLCM may be present in the composition at from about 36% by weight to about 45% by weight, from about 37% by weight to about 45% by weight, from about 38% by weight to about 45% by weight, from about 39% by weight to about 45% by weight, from about 40% by weight to about 45% by weight, from about 41% by weight to about 45% by weight, from about 42% by weight to about 45% by weight, from about 43% by weight to about 45% by weight, or from about 44% by weight to about 45% by weight relative to the total weight of the composition. In some embodiments, the HVLCM may be present in the composition at from about 35% by weight to about 37% by weight, from about 37% by weight to about 39% by weight, from about 39% by weight to about 41% by weight, from about 41% by weight to about 43% by weight, or from about 43% by weight to about 45% by weight relative to the total weight of the composition. In some embodiments, the HVLCM may be present in the composition at about 35% by weight, about 36% by weight, about 37% by weight, about 38% by weight, about 39% by weight, about 40% by weight, about 41% by weight, about 42% by weight, about 43% by weight, about 44% by weight, or about 45% by weight relative to the total weight of the composition.

In some embodiments, the amount of the HVLCM present in the composition is provided relative to the amount of the solvent present in the composition.

In some embodiments, Sucrose Acetate Isobutyrate (“SAIB”) may be included in the composition as the HVLCM. SAIB is a non-polymeric highly viscous liquid at temperatures ranging from −80° C. to over 100° C., it is a fully esterified sucrose derivative, at a nominal ratio of six isobutyrates to two acetates. The chemical structure of SAIB is provided in U.S. Application Publication No. 2009/0215808, the disclosure of which is incorporated by reference herein. The SAIB material is available from a variety of commercial sources including Eastman Chemical Company, where it is available as a mixed ester that does not crystallize but exists as a very highly viscous liquid. It is a hydrophobic, non-crystalline, low molecular weight molecule that is water insoluble and has a viscosity that varies with temperature. For example, pure SAIB exhibits a viscosity of approximately 2,000,000 centipoise (cP) at ambient temperature (RT) and approximately 600 cP at 80° C. The SAIB material has unique solution-viscosity relationship in that a SAIB solution established in a number of organic solvents has a significantly lower viscosity value than the pure SAIB material, and therefore the SAIB-organic solvent solutions render themselves capable of processing using conventional equipment such as mixers, liquid pumps and capsule production machines. SAIB also has applications in drug formulation and delivery, for example as described in U.S. Pat. Nos. 5,747,058; 5,968,542; 6,413,536; and 6,498,153, the disclosure of which are incorporated by reference herein.

In the compositions of the present disclosure, SAIB may be used as the HVLCM and may be present at from about 35% by weight to about 45% by weight, based on total weight of the composition. For example, the SAIB may be present in the composition at from about 36% by weight to about 45% by weight, from about 37% by weight to about 45% by weight, from about 38% by weight to about 45% by weight, from about 39% by weight to about 45% by weight, from about 40% by weight to about 45% by weight, from about 41% by weight to about 45% by weight, from about 42% by weight to about 45% by weight, from about 43% by weight to about 45% by weight, or from about 44% by weight to about 45% by weight relative to the total weight of the composition. In some embodiments, the SAIB may be present in the composition at from about 35% by weight to about 37% by weight, from about 37% by weight to about 39% by weight, from about 39% by weight to about 41% by weight, from about 41% by weight to about 43% by weight, or from about 43% by weight to about 45% by weight relative to the total weight of the composition. In some embodiments, the SAIB may be present in the composition at about 35% by weight, about 36% by weight, about 37% by weight, about 38% by weight, about 39% by weight, about 40% by weight, about 41% by weight, about 42% by weight, about 43% by weight, about 44% by weight, or about 45% by weight relative to the total weight of the composition.

In some embodiments, the amount of SAIB present in the composition is provided relative to the amount of the solvent present in the composition.

In some embodiments, it may be beneficial to provide a SAIB carrier material having a lower peroxide level to avoid peroxide-based degradation of various components of the composition and/or active agent. See, e.g., U.S. Patent Application Publication Number US 2007/0027105, “Peroxide Removal From Drug Delivery Vehicle”, the disclosure of which is incorporated by reference herein.

### Solvent

Solvents may be used in the compositions of the present disclosure to dissolve one or more of the following constituents: HVCLMs; active agents; network formers; rheology modifiers; viscosity enhancing agents; hydrophilic agents; and stabilizing agents. In some embodiments, the solvent can dissolve both the HVLCM and the network former. In addition, materials that can serve as rheology modifiers in certain compositions can also serve the function as a solvent to one or more constituent (e.g., the HVLCM, or the active agent), or serve solely as a solvent in other compositions. One example of such a solvent is IPM, which is a hydrophobic solvent. In one embodiment of the compositions of the present disclosure, therefore, a composition may include both a hydrophilic solvent and a hydrophobic solvent. Organic solvents suitable for use with the compositions of the present disclosure include, but are not limited to: substituted heterocyclic compounds such as N-methyl-2-pyrrolidone (NMP) and 2-pyrrolidone (2-pyrol); triacetin; ethyl lactate, esters of carbonic acid and alkyl alcohols such as propylene carbonate, ethylene carbonate and dimethyl carbonate; fatty acids such as acetic acid, lactic acid and heptanoic acid; alkyl esters of mono-, di-, and tricarboxylic acids such as 2-ethyoxyethyl acetate, ethyl acetate, methyl acetate, ethyl lactate, ethyl butyrate, diethyl malonate, diethyl glutonate, tributyl citrate, diethyl succinate, tributyrin, isopropyl myristate (IPM), dimethyl adipate, dimethyl succinate, dimethyl oxalate, dimethyl citrate, triethyl citrate, acetyl tributyl citrate, glyceryl triacetate; alkyl ketones such as acetone and methyl ethyl ketone; ether alcohols such as 2-ethoxyethanol, ethylene glycol dimethyl ether, glycofurol and glycerol formal; alcohols such as benzyl alcohol, ethanol and propanol; polyhydroxy alcohols such as propylene glycol, polyethylene glycol (PEG), glycerin (glycerol), 1,3-butyleneglycol, and isopropylidene glycol (2,2-dimethyl-1,3-dioxolone-4-methanol); Solketal; dialkylamides such as dimethylformamide, dimethylacetamide; dimethylsulfoxide (DMSO) and dimethylsulfone; tetrahydrofuran; lactones such as ε-caprolactone and butyrolactone; cyclic alkyl amides such as caprolactam; aromatic amides such as N,N-dimethyl-m-toluamide, and 1-dodecylazacycloheptan-2-one; and the like; and mixtures and combinations thereof.

In some embodiments, the solvent is selected from triacetin, ethyl lactate, N-methyl-2-pyrrolidone, 2-pyrrolidone, dimethylsulfoxide, ethyl lactate, propylene carbonate, and glycofurol. In some embodiments, the solvent is triacetin which is a hydrophilic solvent. In some embodiments, the hydrophilic triacetin solvent can be combined with the IPM rheology modifier which is a hydrophobic solvent to provide a solvent hydrophobic/hydrophilic solvent system within the composition. Alternatively, in some embodiments, the solvent is ethyl lactate which is a hydrophilic solvent. In some embodiments, the hydrophilic ethyl lactate solvent can be combined with the IPM rheology modifier which is a hydrophobic solvent to provide a solvent hydrophobic/hydrophilic solvent system within the composition.

The solvent, which can include one or more suitable solvent materials, can be present in the compositions at from about 15% by weight to about 45% by weight, based on total weight of the composition. For example, the solvent may be present in the composition at from about 16% by weight to about 45% by weight, at from about 17% by weight to about 45% by weight, at from about 18% by weight to about 45% by weight, at from about 19% by weight to about 45% by weight, at from about 20% by weight to about 45% by weight, at from about 21% by weight to about 45% by weight, from about 22% by weight to about 45% by weight, from about 23% by weight to about 45% by weight, from about 24% by weight to about 45% by weight, from about 25% by weight to about 45% by weight, from about 26% by weight to about 45% by weight, from about 27% by weight to about 45% by weight, from about 28% by weight to about 45% by weight, from about 29% by weight to about 45% by weight, from about 30% by weight to about 45% by weight, from about 31% by weight to about 45% by weight, from about 32% by weight to about 45% by weight, from about 33% by weight to about 45% by weight, from about 34% by weight to about 45% by weight, from about 35% by weight to about 45% by weight, from about 36% by weight to about 45% by weight, from about 37% by weight to about 45% by weight, from about 38% by weight to about 45% by weight, from about 39% by weight to about 45% by weight, from about 40% by weight to about 45% by weight, from about 41% by weight to about 45% by weight, from about 42% by weight to about 45% by weight, from about 43% by weight to about 45% by weight, from about 44% by weight to about 45% by weight relative to the total weight of the composition. In some embodiments, the solvent may be present in the composition at from about 15% by weight to about 17% by weight, from about 17% by weight to about 19% by weight, from about 19% by weight to about 21% by weight, from about 21% by weight to about 23% by weight, from about 23% by weight to about 25% by weight, from about 25% by weight to about 27% by weight, from about 27% by weight to about 29% by weight, from about 29% by weight to about 31% by weight, from about 31% by weight to about 33% by weight, from about 33% by weight to about 35% by weight, from about 35% by weight to about 37% by weight, from about 37% by weight to about 39% by weight, from about 39% by weight to about 41% by weight, from about 41% by weight to about 43% by weight, or from about 43% by weight to about 45% by weight relative to the total weight of the composition. In some embodiments, the solvent may be present in the composition at about 15% by weight, about 16% by weight, about 17% by weight, about 18% by weight, about 19% by weight about 20% by weight, about 21% by weight, about 22% by weight, about 23% by weight, about 24% by weight, about 25% by weight, about 26% by weight, about 27% by weight, about 28% by weight, about 29% by weight, about 30% by weight, about 31% by weight, about 32% by weight, about 33% by weight, about 34% by weight, about 35% by weight, about 36% by weight, about 37% by weight, about 38% by weight, about 39% by weight, about 40% by weight, about 41% by weight, about 42% by weight, about 43% by weight, about 44% by weight, or about 45% by weight relative to the total weight of the composition.

### Rheology Modifier

Rheology refers to the property of deformation and/or flow of a liquid, and rheology modifiers are used to modify viscosity and flow of a liquid composition. Rheology modifiers, which may be used in the compositions of the present disclosure include, for example, caprylic/capric triglyceride (e.g., Miglyol® 810), isopropyl myristate (IM or IPM), ethyl oleate, triethyl citrate, dimethyl phthalate, and benzyl benzoate. The rheology modifier, which can include one or more suitable rheology modifier materials, and can be present in the compositions at from about 1 to about 20 percent by weight relative to the total weight of the composition (wt %).

In some embodiments, the rheology modifier is or includes IPM. The IPM material is a pharmaceutically acceptable hydrophobic solvent. The rheology modifier, which can include one or more suitable rheology modifier materials, can be present in the compositions at from about 1 to about 20 percent by weight relative to the total weight of the composition (wt %), e.g., at about 1 wt %, at about 2 wt %, at about 3 wt %, at about 4 wt %, at about 5 wt %, at about 6 wt %, at about 7 wt %, at about 8 wt %, at about 9 wt %, at about 10 wt %, at about 11 wt %, at about 12 wt %, at about 13 wt %, at about 14 wt %, at about 15 wt %, at about 16 wt %, at about 17 wt %, at about 18 wt %, at about 19 wt %, or at about 20 wt %.

Alternatively, in some embodiments, the rheology modifier is caprylic/capric triglyceride (e.g., Miglyol® 812). The caprylic/capric triglyceride (e.g., Miglyol® 812) material is a pharmaceutically acceptable hydrophobic solvent. The rheology modifier, which can include one or more suitable rheology modifier materials, can be present in the compositions at from about 1 to about 20 percent by weight relative to the total weight of the composition (wt %), e.g., at about 1 wt %, at about 2 wt %, at about 3 wt %, at about 4 wt %, at about 5 wt %, at about 6 wt %, at about 7 wt %, at about 8 wt %, at about 9 wt %, at about 10 wt %, at about 11 wt %, at about 12 wt %, at about 13 wt %, at about 14 wt %, at about 15 wt %, at about 16 wt %, at about 17 wt %, at about 18 wt %, at about 19 wt %, or at about 20 wt %.

In some embodiments, the rheology modifier is present in the compositions of the present disclosure in an amount relative to the amount of solvent in the compositions.

### Network Former

Network formers may be added to a composition such that, upon exposure to an aqueous environment, they form a three dimensional network within the composition. While not intending to be bound by any particular theory, it is believed that the network former allows the formation of a micro-network within the composition upon exposure to an aqueous environment. This micro-network formation appears to be due, at least in part, to a phase inversion (e.g., a change in glass transition temperature, T_{g}) of the network former. The result is believed to be a skin or surface layer of precipitated network former at the interface between the composition and the aqueous environment of the GI tract, as well as the formation of a three-dimensional micro-network of precipitated network former within the composition. The network former is selected so as to have good solubility in the selected solvent used in the compositions, for example a solubility of between about 0.1 and 20 wt %. Additionally, good network formers will typically have a Log P between about −1 to 7. Suitable network formers include, for example, cellulose acetate butyrate (“CAB”), carbohydrate polymers, organic acids of carbohydrate polymers and other polymers, hydrogels, cellulose acetate phthalate, ethyl cellulose, Pluronic® (nonionic triblock copolymer), Eudragit® (polymethacrylate), Carbomer™ (polyacrylic acid), hydroxyl propyl methyl cellulose, other cellulose acetates such as cellulose triacetate, Poly(methyl methacrylate) (PMMA), as well as any other material capable of associating, aligning or congealing to form three-dimensional networks in an aqueous environment.

In some embodiments, the network former used in the compositions of the present disclosure is or includes a CAB having a number average molecular weight ranging from about 50,000 Daltons to about 100,000 Daltons, e.g., from about 60,000 Daltons to about 100,000 Daltons, from about 70,000 Daltons to about 100,000 Daltons, from about 80,000 Daltons to about 100,000 Daltons, or from about 90,000 Daltons to about 100,000 Daltons. In some embodiments, the network former used in the compositions of the present disclosure is or includes a CAB having a number average molecular weight ranging from about 60,000 Daltons to about 90,000 Daltons, or from about 70,000 Daltons to about 80,000 Daltons. In some embodiments, the network former used in the compositions of the present disclosure is or includes a CAB having a number average molecular weight of about 50,000 Daltons, about 55,000 Daltons, about 60,000 Daltons, about 65,000 Daltons, about 70,000 Daltons, about 75,000 Daltons, about 80,000 Daltons, about 85,000 Daltons, about 90,000 Daltons, about 95,000 Daltons, or about 100,000 Daltons.

In some embodiments, the network former used in the compositions of the present disclosure is or includes a CAB having at least one feature selected from a butyryl content ranging from about 17% to about 41%, an acetyl content ranging from about 13% to about 30%, and a hydroxyl content ranging from about 0.5% to about 1.7%. In some further embodiments, the network former used in the compositions of the present disclosure is or includes a CAB comprising at least two of a butyryl content ranging from about 17% to about 41%, an acetyl content ranging from about 13% to about 30%, and a hydroxyl content ranging from about 0.5% to about 1.7%. In still further embodiments, the network former used in the compositions of the present disclosure is or includes a CAB comprising all three of a butyryl content ranging from about 17% to about 41%, an acetyl content ranging from about 13% to about 30%, and a hydroxyl content ranging from about 0.5% to about 1.7%. In further embodiments, in addition to one of the above features of butyryl content, acetyl content and/or hydroxyl content, the CAB also has a number average molecular weight ranging from about 50,000 Daltons to about 100,000 Daltons, e.g., from about 60,000 Daltons to about 100,000 Daltons, from about 70,000 Daltons to about 100,000 Daltons, from about 80,000 Daltons to about 100,000 Daltons, or from about 90,000 Daltons to about 100,000 Daltons. In further embodiments, in addition to one of the above features of butyryl content, acetyl content and/or hydroxyl content, the CAB also has a number average molecular weight ranging from about 60,000 Daltons to about 90,000 Daltons, or from about 70,000 Daltons to about 80,000 Daltons. In further embodiments, in addition to one of the above features of butyryl content, acetyl content and/or hydroxyl content, the CAB also has a number average molecular weight of about 50,000 Daltons, about 55,000 Daltons, about 60,000 Daltons, about 65,000 Daltons, about 70,000 Daltons, about 75,000 Daltons, about 80,000 Daltons, about 85,000 Daltons, about 90,000 Daltons, about 95,000 Daltons, or about 100,000 Daltons.

Accordingly, in some embodiments, the network former used in the compositions of the present disclosure is or includes a CAB having a butyryl content ranging from about 17% to about 41%. In some embodiments, the network former used in the compositions of the present disclosure is or includes a CAB having an acetyl content ranging from about 13% to about 30%. In some embodiments, the network former used in the compositions of the present disclosure is or includes a CAB having a hydroxyl content ranging from about 0.5% to about 1.7%. In some embodiments, the network former used in the compositions of the present disclosure is or includes a CAB having a butyryl content ranging from about 17% to about 41% and an acetyl content ranging from about 13% to about 30%. In some embodiments, the network former used in the compositions of the present disclosure is or includes a CAB having a butyryl content ranging from about 17% to about 41% and a hydroxyl content ranging from about 0.5% to about 1.7%. In some embodiments, the network former used in the compositions of the present disclosure is or includes a CAB having an acetyl content ranging from about 13% to about 30% and a hydroxyl content ranging from about 0.5% to about 1.7%. In still other embodiments, the network former used in the compositions of the present disclosure is or includes a CAB having a butyryl content ranging from about 17% to about 41%, an acetyl content ranging from about 13% to about 30%, and a hydroxyl content ranging from about 0.5% to about 1.7%.

In some embodiments, the network former used in the compositions of the present disclosure is or includes cellulose acetate butyrate grade 381-20BP (“CAB 381-20BP” available from Eastman Chemicals). In some embodiments, the network former used in the compositions of the present disclosure is or includes a CAB, wherein the CAB is a non-biodegradable polymer material that has the following chemical and physical characteristics: butyryl content of about 36 wt %, acetyl content of about 15.5 wt %, hydroxyl content of about 0.8%, a melting point of from about 185-196° C., a glass transition temperature of about 128° C., and a number average of from about 66,000 to 83,000, e.g., about 70,000. In some embodiments, if a CAB material is used in the composition, it may be subjected to an ethanol washing step (and subsequent drying step) prior to addition to the formulation in order to remove potential contaminants therefrom.

In some embodiments, the network former of the present disclosure specifically excludes a network former having an acetyl content of about 2.0%, a butyryl content of about 46.0%, a hydroxyl content of 4.8%, a melting point of from about 150-160° C., a glass transition temperature of about 136° C., and a number average molecular weight of about 20,000, e.g., CAB-553-0.4 available from Eastman Chemicals).

In some embodiments, the network former of the present disclosure specifically excludes a network former, e.g, a CAB, which is soluble in ethanol.

The network former, which can include one or more suitable network former materials, can be present in the compositions at from about 0.1 to about 20 percent by weight relative to the total weight of the composition (wt %), e.g., at from about 1 to about 18 wt %, from about 2 to about 10 wt %, from about 4 to about 6 wt %, or at about 5 wt %. In some embodiments, a network former is present in the compositions of the present disclosure at from about 0.1 to about 1 wt %, about 1 to about 5 wt %, about 5 to about 10 wt %, about 10 to about 15 wt %, or about 15 to about 20 wt %. In some embodiments, a network former is present in the compositions of the present disclosure at about 0.1 wt %, about 0.5 wt %, about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, or about 20 wt %.

### Hydrophilic Agent

Materials that can be used as “hydrophilic agents” in the compositions of the present disclosure include those that have natural affinity for aqueous systems. A material may be regarded as a hydrophilic agent for the purposes of this disclosure if the material displays a water sorption between about 10 to 100% (w/w). Hydrophilic agents will have a low Log P value, for example, a Log P of less than +1. As discussed herein above, there are a number of constituents which may be used to produce the compositions of the present disclosure that can be classed as a hydrophilic material (e.g., a hydrophilic solvent), or at least a material having a hydrophilic portion (e.g., a rheology modifier). Since the HVLCM material used in the compositions is hydrophobic, it may be useful to include other materials in the composition that are hydrophilic in order to provide a carrier system that is balanced to have both hydrophobic and hydrophilic characteristics. For example, it is believed that the inclusion of one or more hydrophilic agents in the compositions of the present disclosure may participate in the control of active agent diffusion from the compositions. Accordingly, suitable hydrophilic agents include, but are not limited to, sugars such as sorbitol, lactose, mannitol, fructose, sucrose and dextrose, salts such as sodium chloride and sodium carbonate, starches, hyaluronic acid, glycine, fibrin, collagen, polymers such as hydroxylpropylcellulose (“HPC”), carboxymethylcellulose, hydroxyethyl cellulose (“HEC”); polyethylene glycol and polyvinylpyrrolidone, and the like. In some embodiments, a controlled release carrier system is provided that includes HEC as a hydrophilic agent.

The hydrophilic agent, which can include one or more suitable hydrophilic agent materials, e.g., HEC, can be present in the compositions at from about 0.1 to about 10 percent by weight relative to the total weight of the composition (wt %), e.g., from about 1 to about 8 wt %, from about 2 to about 7 wt %, from about 3 to about 6 wt %, or from about 4 to about 5 wt %. In some embodiments, a hydrophilic agent is present in the compositions of the present disclosure at about 0.1 wt % to about 0.5 wt %, about 0.5 wt % to about 1 wt %, about 1 wt % to about 5 wt %, or about 5 wt % to about 10 wt %. In some embodiments, a hydrophilic agent is present in the compositions of the present disclosure at about 0.1 wt %, about 0.5 wt %, about 1 wt %, about 2 wt %, about 3 wt %, about 4 wt %, about 5 wt %, about 6 wt %, about 7 wt %, about 8 wt %, about 9 wt %, or about 10 wt %.

### Viscosity Enhancing Agent

Viscosity enhancing agents (e.g., thixotropic thickening agents) can be selected to have good hydrogen bonding capability, such as a bonding capability greater than or equal to one per molecule. In certain cases, the viscosity enhancing agent has very low to no significant solubility in the composition. If the agent is soluble, then, in some embodiments, the solubility is less than 50 wt %. For inorganic or mineral viscosity enhancing agents, it is preferable if the material has a specific surface area greater than or equal to about 100 m²/g. Suitable viscosity enhancing agents include biodegradable and non-biodegradable polymer materials. Non-limiting examples of suitable biodegradable polymers and oligomers include: poly(lactide), poly(lactide-co-glycolide), poly(glycolide), poly(caprolactone), polyamides, polyanhydrides, polyamino acids, polyorthoesters, polycyanoacrylates, poly(phosphazines), poly(phosphoesters), polyesteramides, polydioxanones, polyacetals, polyketals, polycarbonates, polyorthocarbonates, degradable polyurethanes, polyhydroxybutyrates, polyhydroxyvalerates, polyalkylene oxalates, polyalkylene succinates, poly(malic acid), chitin, chitosan, and copolymers, terpolymers, oxidized cellulose, hydroxyethyl cellulose, or combinations or mixtures of the above materials. Suitable non-biodegradable polymers include: polyacrylates, ethylene-vinyl acetate polymers, cellulose and cellulose derivatives, acyl substituted cellulose acetates and derivatives thereof including cellulose acetate butyrate (CAB), which is also used herein as a network former, non-erodible polyurethanes, polystyrenes, polyvinyl chloride, polyvinyl fluoride, polyvinyl (imidazole), chlorosulphonated polyolefins, polyethylene oxide, and polyethylene. In some embodiments, a viscosity enhancing agent includes mixtures of esters of acids and hydroxyacids.

Other suitable viscosity enhancing materials include mineral particles such as clay compounds, including, talc, bentonite and kaolin; metal oxides including silicon dioxide, zinc oxide, magnesium oxide, titanium oxide, and calcium oxide; and fumed silica, reagent grade sand, precipitated silica, amorphous silica, colloidal silicon dioxide, fused silica, silica gel, and quartz. In some embodiments of the present disclosure, a colloidal silicon dioxide (Cab-o-Sil®) is used in the compositions as a viscosity enhancing agent. In some embodiments, cetyl alcohol or carnauba wax is used in the compositions as a viscosity enhancing agent.

The viscosity enhancing agent, e.g., mineral particle, which can include one or more suitable viscosity enhancing materials, can be present in the formulations at from about 2.4 to about 6.0 percent by weight relative to the total weight of the composition (wt %), e.g., at from about 2.5 to about 6.0 wt %, at from about 2.6 to about 6.0 wt %, at from about 2.7 to about 6.0 wt %, at from about 2.8 to about 6.0 wt %, at from about 2.9 to about 6.0 wt %, at from about 3.0 to about 6.0 wt %, at from about 3.1 to about 6.0 wt %, at from about 3.2 to about 6.0 wt %, at from about 3.3 to about 6.0 wt %, at from about 3.4 to about 6.0 wt %, at from about 3.5 to about 6.0 wt %, at from about 3.6 to about 6.0 wt %, at from about 3.7 to about 6.0 wt %, at from about 3.8 to about 6.0 wt %, at from about 3.9 to about 6.0 wt %, at from about 4.0 to about 6.0 wt %, at from about 4.1 to about 6.0 wt %, at from about 4.2 to about 6.0 wt %, at from about 4.3 to about 6.0 wt %, at from about 4.4 to about 6.0 wt %, at from about 4.5 to about 6.0 wt %, at from about 4.6 to about 6.0 wt %, at from about 4.7 to about 6.0 wt %, at from about 4.8 to about 6.0 wt %, at from about 4.9 to about 6.0 wt %, at from about 5.0 to about 6.0 wt %, at from about 5.1 to about 6.0 wt %, at from about 5.2 to about 6.0 wt %, at from about 5.3 to about 6.0 wt %, at from about 5.4 to about 6.0 wt %, at from about 5.5 to about 6.0 wt %, at from about 5.6 to about 6.0 wt %, at from about 5.7 to about 6.0 wt %, at from about 5.8 to about 6.0 wt %, or at from about 5.9 to about 6.0 wt %.

In some embodiments, a composition according to the present disclosure includes a viscosity enhancing agent, e.g., mineral particle, at from about 2.0 to about 3.0 percent by weight relative to the total weight of the composition. In some embodiments, a composition according to the present disclosure includes a viscosity enhancing agent, e.g., mineral particle, at from about 2.0 to about 2.2 wt %, at from about 2.2 wt % to about 2.4 wt %, at from about 2.4 wt % to about 2.6 wt %, at from about 2.6 wt % to about 2.8 wt %, or at from about 2.8 wt % to about 3.0 wt %.

In some embodiments, a composition according to the present disclosure includes a viscosity enhancing agent, e.g., mineral particle (e.g., silicon dioxide) at about 2.0 wt %, about 2.1 wt %, about 2.2 wt %, about 2.3 wt %, about 2.4 wt %, about 2.5 wt %, about 2.6 wt %, about 2.7 wt %, about 2.8 wt %, about 2.9 wt %, or about 3.0 wt %.

As discussed in the Examples below, providing a viscosity enhancing agent, e.g., a mineral particle such as silicon dioxide, in an amount outside of one or more of the ranges specified above may result in undesirable composition characteristics. For example, variability in a dissolution profile of the active agent from the composition, e.g., as evidenced by increased inter-capsule variability, may be seen at relatively low silicon dioxide levels. On the other hand, reduced processability may be seen at relatively high silicon dioxide levels due to an increase in the rigidity and/or viscosity of the composition. Accordingly, in some embodiments, the compositions of the present disclosure specifically exclude viscosity enhancing agents, e.g., mineral particles, in an amount outside of one or more of the ranges specified above.

In some embodiments an unexpected, beneficial balance between dissolution variability and processability may be achieved by including the viscosity enhancing agent, e.g., mineral particle such as silicon dioxide, at from about 2.4 to about 5.4 percent by weight relative to the total weight of the composition (wt %), e.g., at from about 2.4 to about 2.6 wt %, at from about 2.6 to about 2.8 wt %, at from about 2.8 to about 3.0 wt %, at from about 3.0 to about 3.2 wt %, at from about 3.2 to about 3.4 wt %, at from about 3.4 to about 3.6 wt %, at from about 3.6 to about 3.8 wt %, at from about 3.8 to about 4.0 wt %, at from about 4.0 to about 4.2 wt %, at from about 4.2 to about 4.4 wt %, at from about 4.4 to about 4.6 wt %, at from about 4.6 to about 4.8 wt %, at from about 4.8 to about 5.0 wt %, at from about 5.0 to about 5.2 wt %, or at from about 5.2 to about 5.4 wt %. Similarly, a beneficial balance between dissolution variability and processability may be achieved by including the viscosity enhancing agent, e.g., mineral particle such as silicon dioxide, at from about 2.6 to about 5.4 wt %, at from about 2.8 to about 5.4 wt %, at from about 3.0 to about 5.4 wt %, at from about 3.2 to about 5.4 wt %, at from about 3.4 to about 5.4 wt %, at from about 3.6 to about 5.4 wt %, at from about 3.8 to about 5.4 wt %, at from about 4.0 to about 5.4 wt %, at from about 4.2 to about 5.4 wt %, at from about 4.4 to about 5.4 wt %, at from about 4.6 to about 5.4 wt %, at from about 4.8 to about 5.4 wt %, at from about 5.0 to about 5.4 wt %, or at from about 5.2 to about 5.4 wt %.

As discussed above, a viscosity enhancing agent, e.g., mineral particle, such as silicon dioxide, cetyl alcohol or carnauba wax, when included at specific concentration ranges in the compositions of the present disclosure, may reduce dissolution variability of the composition, e.g., inter-capsule dissolution variability as determined using a USP Apparatus 2 dissolution tester and method as described below in the Examples. See also, USP-NF, Dissolution <711>. Rockville, Md.: US Pharmacopeial Convention; 2008, the disclosure of which is incorporated by reference herein.

### Stabilizing Agent

Materials that can be used as stabilizing agents in the compositions of the present disclosure include any material or substance that can inhibit or reduce degradation (e.g., by chemical reactions) of other substances or substances in the composition with which the stabilizer is mixed. Exemplary stabilizers typically are antioxidants that prevent oxidative damage and degradation, e.g., sodium citrate, ascorbyl palmitate, vitamin A, and propyl gallate and/or reducing agents. Other examples include ascorbic acid, vitamin E, sodium bisulfite, butylhydroxyl toluene (BHT), BHA, acetylcysteine, monothioglycerol, phenyl-alpha-nathylamine, lecithin, and EDTA. These stabilizing materials, which can include one or more of such suitable materials, can be present in the compositions at from about 0.001 to about 2 percent by weight relative to the total weight of the composition (wt %), e.g., at from about 0.01 to about 0.1 wt %, or at from about 0.01 to about 0.02 wt %. In some embodiments, the compositions of the present disclosure specifically exclude a stabilizing agent, such as those listed above.

### Surfactants

In some embodiments, a composition according to the present disclosure may include one or more surfactants. Materials that can be used as surfactants in the practice of the present disclosure include neutral and/or anionic/cationic excipients. Accordingly, suitable charged lipids include, without limitation, phosphatidylcholines (lecithin), and the like. Detergents will typically be a nonionic, anionic, cationic or amphoteric surfactant. Examples of suitable surfactants include, for example, Tergitol® and Triton® surfactants (Union Carbide Chemicals and Plastics); polyoxyethylenesorbitans, e.g., TWEEN® surfactants (Atlas Chemical Industries); polysorbates; polyoxyethylene ethers, e.g. Brij; pharmaceutically acceptable fatty acid esters, e.g., lauryl sulfate and salts thereof; ampiphilic surfactants (glycerides, etc.); Gelucire®s (saturated polyglycolized glyceride (e.g., Gattefosse brand); and like materials. Surfactants, which can include one or more suitable surfactant material, can be present in the compositions of the present disclosure at from about 0.01 to about 5 percent by weight relative to the total weight of the composition (wt %), e.g., at from about 0.1 to about 5 wt %, or at from about 0.1 to about 3 wt %. In some embodiments, a surfactant is present in the compositions of the present disclosure at about 0.1 wt %, about 0.5 wt %, about 1 wt %, about 2 wt %, about 3 wt %, about 4 wt %, or about 5 wt %.

In some embodiments, a suitable surfactant for incorporation into the compositions of the present disclosure includes one or more Gelucire® s (saturated polyglycolized glycerides). Suitable Gelucire® s include, e.g., Gelucire® 44/14 (lauroyl polyoxylglycerides) and Gelucire® 50/13 (stearoyl polyoxylglycerides). Accordingly, in some embodiments, a Gelucire® e.g., Gelucire® 44/14, Gelucire® 50/13, or a combination thereof, is present the compositions of the present disclosure at from about 0.01 to about 5 percent by weight relative to the total weight of the composition (wt %), e.g., at from about 0.1 to about 5 wt %, or at from about 0.1 to about 3 wt %. In some embodiments, a Gelucire® e.g., Gelucire® 44/14, Gelucire® 50/13, or a combination thereof, is present in the compositions of the present disclosure at about 0.1 wt %, about 0.5 wt %, about 1 wt %, about 2 wt %, about 3 wt %, about 4 wt %, or about 5 wt %.

### Exemplary Compositions

With reference to the various components discussed above, exemplary compositions are now described. In some embodiments a composition is provided which includes a pharmacologically active agent; about 15% by weight to about 45% by weight, based on total weight of the composition, of a solvent; and about 1% by weight to about 20% by weight, based on total weight of the composition, of a rheology modifier. Optionally, the composition may be provided within a capsule having a water content of less than about 10% by weight, e.g., an HPMC capsule having a water content of less than about 10% by weight, e.g., less than about 5% by weight.

In some embodiments a composition is provided which includes a pharmacologically active agent; about 18% by weight to about 27% by weight, based on total weight of the composition, of a solvent; and about 14% by weight to about 19% by weight, based on total weight of the composition, of a rheology modifier. Optionally, the composition may be provided within a capsule having a water content of less than about 10% by weight, e.g., an HPMC capsule having a water content of less than about 10% by weight, e.g., less than about 5% by weight.

In some embodiments, compositions are provided which include a pharmacologically active agent; a solvent; a network former; and a mineral particle, wherein the mineral particle is present in the composition in an amount from about 1.9% by weight to about 3.0% by weight relative to the total weight of the composition. The mineral particles may be selected from silicon dioxide, cetyl alcohol, or carnauba wax. Optionally, the composition may be provided within a capsule having a water content of less than about 10% by weight, e.g., an HPMC capsule having a water content of less than about 10% by weight, e.g., less than about 5% by weight.

In some embodiments a composition is provided which includes a pharmacologically active agent; about 18% by weight to about 27% by weight, based on total weight of the composition, of a solvent; and about 14% by weight to about 19% by weight, based on total weight of the composition, of a rheology modifier; and a mineral particle, wherein the mineral particle is present in the composition in an amount from about 1.9% by weight to about 3.0% by weight relative to the total weight of the composition. The mineral particles may be selected from silicon dioxide, cetyl alcohol, or carnauba wax. Optionally, the composition may be provided within a capsule having a water content of less than about 10% by weight, e.g., an HPMC capsule having a water content of less than about 10% by weight, e.g., less than about 5% by weight.

### Methods of Making, Encapsulating and Administering

Once constituents have been selected to produce a composition (e.g., an extended release composition) in accordance with the present disclosure, a liquid pharmaceutical formulation can be prepared by simply mixing, for example a HVLCM, a rheology modifier, a network former, the active agent, a solvent and any additional additives. The compositions of the present disclosure are produced as liquid mixtures, and have a number of excipient ingredients that are in solution, suspension, or in partial solution within the final formulation. Suitable methods for compounding or manufacturing the formulations make use of typical pharmaceutical/chemical mixing and handling apparatus and techniques. Since the liquid formulations of the present disclosure are formed from a number of highly viscous liquids and solids, they may have high final viscosities. Accordingly, the specific equipment and techniques employed in the manufacture of such formulations may be selected so as to accommodate such material demands. In particular, various excipients, such as network formers, may be added to the formulation mixture in the solid or semi-solid state, and as such they may be screened or otherwise size-reduced prior to addition to a formulation mixing apparatus. Other solid excipients may require melting prior to addition to the liquid mixture. The HVLCM materials are very high viscosity liquid materials, however they tend to exhibit a dramatic reduction in viscosity with increases in heat, and as such the mixing apparatus may be heated to accommodate the addition of the HVLCM material or other similar materials. However, the mixing and processing conditions should take into account the final integrity of the formulation and accordingly the mixing conditions may be selected so as to have a low-sheer effect on the formulation, and/or to avoid any extended or pronounced excursions into high or low heat conditions. Once the formulation has been properly combined, an appropriate amount of the resulting liquid mixture can be placed into a suitable capsule, such as a gelatin or HPMC capsule to provide an oral pharmaceutical dosage form. Alternative liquid formulations may include emulsifying the mixture in water, and introducing this emulsion into a capsule.

In some embodiments, an oral dosage form is provided which is composed of a liquid formulation containing the active agent and any additional components within an enclosure or capsule, e.g., a biodegradable enclosure or capsule, such as a capsule or a gelatin capsule (“gelcap”), wherein the capsule is made of a substance that degrades or otherwise dissociates when exposed to conditions present in the gastro-intestinal tract of a mammal. Capsules and gelcaps are well known in drug delivery technology and one of skill could select such a capsule as appropriate for delivery of a particular active agent. Once the capsule has dissolved or dissociated from the composition, the disclosed compositions generally remains intact, especially for hydrophobic formulations, and passes through the GI tract without emulsification or fragmentation.

Suitable capsules which may be utilized in connection with the disclosed compositions include, but are not limited to hard-shelled capsules, soft-shelled capsules, and interlocking capsules.

In some embodiments a suitable capsule includes gelatin or synthetic polymers such as hydroxyl ethyl cellulose and hydroxyl propylmethyl cellulose. Gelcaps can be of the hard or soft variety, including, for example, polysaccharide or hypromellose acetate succinate based caps (e.g., Vegicaps brand, available from Catalent). The capsule can also be coated with an enteric coating material such as AQIAT (Shin-Etsu) to delay release.

As discussed in the Examples below, certain time-dependent changes in drug release performance have been observed for Reference Formulation A. Without intending to be bound by any particular theory, it is believed that reducing the amount of water available to the compositions of the present disclosure may minimize these effects. For example, by utilizing HPMC capsules (˜2-6% w/w water) instead of gelatin capsules (˜13-15% w/w water) the amount of water available to the compositions may be reduced. Accordingly, in some embodiments, the compositions of the present disclosure are specifically encapsulated in capsules having lower water content than gelatin capsules, e.g., water content of less than about 15% w/w, less than about 14% w/w, less than about 13% w/w, less than about 12% w/w, less than about 11% w/w, less than 10% w/w, less than about 9% w/w, less than about 8% w/w, less than about 7% w/w, less than about 6% w/w, less than about 5% w/w, less than about 4% w/w, less than about 3% w/w, less than about 2% w/w, or less than about 1% w/w. In some embodiments, the compositions of the present disclosure are encapsulated in capsules having a water content of from about 1% w/w to about 10% w/w, e.g., from about 1% w/w to about 9% w/w, from about 1% w/w to about 8% w/w, from about 1% w/w to about 7% w/w, from about 1% w/w to about 6% w/w, from about 1% w/w to about 5% w/w, from about 1% w/w to about 4% w/w, from about 1% w/w to about 3% w/w, or from about 1% w/w to about 2% w/w. In some embodiments, the compositions of the present disclosure are encapsulated in capsules having a water content less than about 1% w/w including, for example, from about 0.1% w/w to about 1% w/w, from about 0.2% w/w to about 0.8% w/w, from about 0.4% w/w to about 0.8% w/w, or from about 0.6% w/w to about 0.8% w/w. Suitable HPMC capsules may include, for example, V-Caps™ and V-caps Plus™.

The water content of a capsule, composition, or composition in combination with a capsule, when provided within a capsule as described in the present disclosure, may be determined by Karl Fischer titration method as set forth in USP <921> Method 1C. In some embodiments, an AquaStar C3000 Karl Fischer Coulometric Titrator may be used in connection with the disclosed titration method.

In some embodiments, a composition according to the present disclosure is one which has relatively low water content. For example, in some embodiments, a composition according to the present disclosure does not include more than about 5% water by weight, based on total weight of the composition. For example, the composition may include water at less than about 5% by weight, less than about 4% by weight, less than about 3% by weight, or less than about 2% by weight, based on the total weight of the composition. In some embodiments, a composition according to the present disclosure includes water at from about 1.0 to about 5.0% by weight, based on total weight of the composition, e.g., at from about 1.0 to about 4.5% by weight, at from about 1.0 to about 3.0% by weight, at from about 1.0 to about 2.5% by weight, at from about 1.0 to about 2.0% by weight, or at from about 1.0 to about 1.5% by weight, based on total weight of the composition. In some embodiments, a composition according to the present disclosure includes water at about 1.0% by weight, about 1.5% by weight, about 2% by weight, about 2.5% by weight, about 3% by weight, about 3.5% by weight, about 4% by weight, about 4.5% by weight, or about 5% by weight, based on the total weight of the composition. The water content of a composition as described in the present disclosure may be determined by Karl Fischer titration method as set forth in USP <921> Method 1C. In some embodiments, an AquaStar C3000 Karl Fischer Coulometric Titrator may be used in connection with the disclosed titration method.

In some embodiments, the water content of the composition and the capsule combined is less than about 5% by weight based on the total weight of the composition and the capsule combined, e.g., less than about 4% by weight, less than about 3% by weight, or less than about 2% by weight based on the total weight of the composition and the capsule combined. In some embodiments, the water content of the composition and the capsule combined is from about 5% by weight to about 4% by weight, from about 4% by weight to about 3% by weight, from about 3% by weight to about 2% by weight, or from about 2% by weight to about 1% by weight based on the total weight of the composition and the capsule combined. In some embodiments, the water content of the composition and the capsule combined is about 1.0% by weight, about 1.5% by weight, about 2% by weight, about 2.5% by weight, about 3% by weight, about 3.5% by weight, about 4% by weight, about 4.5% by weight, or about 5% by weight, based on the total weight of the composition and the capsule combined. The water content of a composition and capsule combined as described in the present disclosure may be determined by Karl Fischer titration method as set forth in USP <921> Method 1C. In some embodiments, an AquaStar C3000 Karl Fischer Coulometric Titrator may be used in connection with the disclosed titration method.

The time-dependent change in release performance may also be addressed by formulating the various components of the composition in specific concentration ranges and/or at specific ratios for oral dosage forms. Accordingly, the present disclosure provides a method of orally administering a composition, including: reducing a time-dependent change in an in vitro release profile of a composition by formulating the composition to include, in addition to a pharmacologically active agent, about 15% by weight to about 45% by weight, based on total weight of the composition, of a solvent, about 1% by weight to about 20% by weight, based on total weight of the composition, of a rheology modifier, and orally administering the composition. Optionally, the composition may be provided within a capsule having a water content of less than about 10% by weight, e.g., an HPMC capsule having a water content of less than about 10% by weight, e.g., less than about 5% by weight.

In some embodiments, the present disclosure provides a method of orally administering a composition, including: reducing a time-dependent change in an in vitro release profile of a composition by formulating the composition to include, in addition to a pharmacologically active agent, a solvent; about 1% by weight to about 20% by weight, based on total weight of the composition, of a rheology modifier, and orally administering the composition. Optionally, the composition may be provided within a capsule having a water content of less than about 10% by weight, e.g., an HPMC capsule having a water content of less than about 10% by weight, e.g., less than about 5% by weight.

In some embodiments, the present disclosure provides a method of orally administering a composition, including: reducing a time-dependent change in an in vitro release profile of a composition by formulating the composition to include, in addition to a pharmacologically active agent, a solvent; a rheology modifier; and orally administering the composition. Optionally, the composition may be provided within a capsule having a water content of less than about 10% by weight, e.g., an HPMC capsule having a water content of less than about 10% by weight, e.g., less than about 5% by weight.

In some embodiments, a method of orally administering a composition is provided which includes: improving reproducibility of an in vitro release profile of a composition by including about 1.9% by weight to about 3.0% by weight, relative to the total weight of the composition, of mineral particle in the composition, wherein the composition also includes a pharmacologically active agent, a solvent, and a network former; and orally administering the composition. Optionally, the composition may be provided within a capsule having a water content of less than about 10% by weight, e.g., an HPMC capsule having a water content of less than about 10% by weight, e.g., less than about 5% by weight.

In some embodiments, a method of orally administering a composition is provided which includes: decreasing the variability of an in vitro release profile of a composition by including about 1.9% by weight to about 3.0% by weight, relative to the total weight of the composition, of mineral particle in the composition, wherein the composition also includes a pharmacologically active agent, a solvent, and a network former; and orally administering the composition. Optionally, the composition may be provided within a capsule having a water content of less than about 10% by weight, e.g., an HPMC capsule having a water content of less than about 10% by weight, e.g., less than about 5% by weight.

In some embodiments, a method of orally administering a composition is provided which includes: forming a composition comprising: a pharmacologically active agent, a solvent, a network former, and mineral particle, wherein the mineral particle is present in the composition in an amount from about 1.9% by weight to about 3.0% by weight relative to the total weight of the composition; improving an in vitro release profile of the composition by encapsulating the composition within a capsule comprising hydroxypropylmethylcellulose to form an encapsulated composition; and orally administering the encapsulated composition. Optionally, the composition may be provided within a capsule having a water content of less than about 10% by weight, e.g., an HPMC capsule having a water content of less than about 10% by weight, e.g., less than about 5% by weight.

In some embodiments, a method of orally administering a composition is provided which includes: forming a composition comprising: a pharmacologically active agent, a solvent, a network former, and mineral particle, wherein the mineral particle is present in the composition in an amount from about 1.9% by weight to about 3.0% by weight relative to the total weight of the composition; reducing exposure of the composition to water by encapsulating the composition within a capsule comprising hydroxypropylmethylcellulose to form an encapsulated composition; and orally administering the encapsulated composition.

In some embodiments, the methods of the present disclosure are suitable for the treatment of pain in a subject. Accordingly, in some embodiments, the present disclosure provides a method for treating pain in a subject, the method comprising: orally administering to the subject a composition comprising an opioid; a solvent; a network former; and a mineral particle, e.g., silicon dioxide, cetyl alcohol, or carnauba wax, wherein the mineral particle is present in the composition in an amount from about 1.9% by weight to about 3.0% by weight relative to the total weight of the composition, wherein the composition is formulated for oral administration, and one or more symptoms or signs associated with the subject's pain is alleviated. Optionally, the composition may be provided within a capsule having a water content of less than about 10% by weight, e.g., an HPMC capsule having a water content of less than about 10% by weight, e.g., less than about 5% by weight.

In certain embodiments, the compositions of the present disclosure may be formulated so as to produce particular controlled plasma levels of an active agent over a particular period, e.g., to maintain a plasma level within an appropriate therapeutic range. An appropriate therapeutic range will vary depending on the active agent, but can range from femtogram/mL levels up to above microgram/mL levels for a desired period of time. For example, a single dose of a composition disclosed herein may result in maintenance of plasma levels of greater than 5 ng/mL for a period of greater than 8 hours. In other embodiments, the plasma level achieved using a single dose may be greater than about 5 ng/mL for a period of greater than about 10 hours, greater than about 12 hours, greater than about 14 hours, greater than about 16 hours, greater than about 18 hours, or greater than about 20 hours. In yet other embodiments, the plasma level achieved using a single dose may be greater than about 5 ng/mL, greater than about 10 ng/mL, greater than about 15 ng/mL, greater than about 20 ng/mL, greater than about 30 ng/mL, greater than about 40 ng/mL, or greater than about 50 ng/mL for a period of about 4, about 8, about 10, about 12, about 14, about 16, about 18, about 20 or about 24 hours. The maximum plasma concentration of an active agent may be reached at a time following administration from between about 0.1 hr to about 24 hr, or from about 0.25 hr to about 10 hr, or from about 0.25 hr to about 8 hr, or from about 0.5 hr to about 6 hr, or from about 0.5 hr to about 4 hr, or from about 0.5 hr to about 2 hr, or from about 0.5 hr to about 1 hr. The time to maximum plasma concentration may be adjusted by adjusting various components of the controlled release carrier system as taught herein.

The plasma levels obtained may be adjusted by adjusting the dose of the active agent, and/or by adjusting the components of the composition, and desirable plasma levels will depend on the therapeutic range or its index for any particular active agent. It is readily within the skill of one in the art to determine the desired therapeutic index.

The rate of active agent release from the composition may be varied depending on the agent used and the dosage required. Release rates may be different in different parts of the GI tract, and release rates may be averaged over the time of transit through the GI tract (approximately 8-24 hrs). Typical average release rates may vary substantially. For many active agents, they may range from about 0.01 to about 500 mg/hr, e.g., from about 0.5 to about 250 mg/hr, from about 0.75 to about 100 mg/hr, from about 1 to about 100 mg/hr, from about 2 to about 100 mg/hr, from about 5 to about 100 mg/hr, from about 10 to about 100 mg/hr, from about 10 to about 80 mg/hr, from about 20 to about 50 mg/hr, or from about 20 to about 40 mg/hr.

Dosage regimens for a particular active agent of interest may be determined by the physician in accordance with standard practices. Once per day (QD) or twice per day (BID) dosing may be used to maintain a sufficient clinical effect, e.g., to maintain pain relief.

### Exemplary Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-94 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below.
1. A composition comprising:

a pharmacologically active agent;

about 15% by weight to about 45% by weight, based on total weight of the composition, of a solvent; and

about 1% by weight to about 20% by weight, based on total weight of the composition, of a rheology modifier.
2. The composition of 1, wherein the solvent is a hydrophilic solvent.
3. The composition of any one of 1-2, wherein the composition is within a hydroxypropylmethylcellulose (HPMC) capsule.
4. The composition of any one of 1-3, wherein the solvent is triacetin, and the rheology modifier is isopropyl myristate (IPM).
5. The composition of any one of 1-4, wherein the solvent is ethyl lactate, and the rheology modifier is isopropyl myristate (IPM).
6. The composition of any one of 1-5, comprising a mineral particle.
7. The composition of 6, wherein the mineral particle comprises silicon dioxide.
8. The composition of any one of 1-7, wherein the pharmacologically active agent is selected from opioid, stimulant, and depressant.
9. The composition of 8, wherein the pharmacologically active agent is an opioid.
10. The composition of 9, wherein the pharmacologically active agent is selected from oxycodone, oxymorphone, hydrocodone, and hydromorphone, either in the free base form or a pharmaceutically acceptable salt form thereof.
11. The composition of 10, wherein the pharmacologically active agent is oxycodone.
12. The composition of any one of 1-11, wherein the composition does not comprise more than 5% water by weight, based on total weight of the composition.
13. The composition of any one of 1-12, wherein the composition comprises water at from about 1.0 to about 2.5% by weight, based on total weight of the composition.
14. A composition comprising:

a pharmacologically active agent;

a solvent;

about 1% by weight to about 20% by weight, based on total weight of the composition, of a rheology modifier.
15. The composition of 14, wherein the solvent is a hydrophilic solvent.
16. The composition of any one of 14-15, wherein the composition is within a hydroxypropylmethylcellulose (HPMC) capsule.
17. The composition of any one of 14-16, wherein the the solvent is triacetin, and the rheology modifier is isopropyl myristate (IPM).
18. The composition of any one of 14-17, wherein the solvent is ethyl lactate, and the rheology modifier is isopropyl myristate (IPM).
19. The composition of any one of 14-18, comprising a mineral particle.
20. The composition of 19, wherein the mineral particle comprises silicon dioxide.
21. The composition of any one of 14-20, wherein the pharmacologically active agent is selected from opioid, stimulant, and depressant.
22. The composition of 21, wherein the pharmacologically active agent is an opioid.
23. The composition of 22, wherein the pharmacologically active agent is a mu opioid agonist.
24. The composition of 23, wherein the pharmacologically active agent is selected from oxycodone, oxymorphone, hydrocodone, and hydromorphone, either in the free base form or a pharmaceutically acceptable salt form thereof.
25. A method of orally administering a composition, comprising:

reducing a time-dependent change in an in vitro release profile of a composition by formulating the composition to include, in addition to a pharmacologically active agent,
about 15% by weight to about 45% by weight, based on total weight of the composition, of a solvent,
about 1% by weight to about 20% by weight, based on total weight of the composition, of a rheology modifier, and
orally administering the composition.
26. The method of 25, wherein the solvent is a hydrophilic solvent.
27. The method of any one of 25-26, wherein the composition is within a hydroxypropylmethylcellulose (HPMC) capsule.
28. The method of any one of 25-27, wherein the solvent is triacetin, and the rheology modifier is isopropyl myristate (IPM).
29. The method of any one of 25-28, wherein the solvent is ethyl lactate, and the rheology modifier is isopropyl myristate (IPM).
30. The method of any one of 25-29, wherein the composition comprises a mineral particle.
31. The method of 30, wherein the mineral particle comprises silicon dioxide.
32. The method of any one of 25-29, wherein the composition comprises a viscosity enhancing agent.
33. The method of 32, wherein the viscosity enhancing agent comprises silicon dioxide, carnauba wax, or cetyl alcohol.
34. The method of any one of 25-33, wherein the pharmacologically active agent is selected from opioid, stimulant, and depressant.
35. The method of 34, wherein the pharmacologically active agent is an opioid.
36. The composition of 34, wherein the pharmacologically active agent is a mu opioid agonist.
37. The composition of 34, wherein the pharmacologically active agent is selected from oxycodone, oxymorphone, hydrocodone, and hydromorphone, either in the free base form or a pharmaceutically acceptable salt form thereof.
38. A method of orally administering a composition, comprising:

reducing a time-dependent change in an in vitro release profile of a composition by formulating the composition to include, in addition to a pharmacologically active agent,
a solvent;
about 1% by weight to about 20% by weight, based on total weight of the composition, of a rheology modifier, and
orally administering the composition.
39. The method of 38, wherein the solvent is a hydrophilic solvent.
40. The method of any one of 38-39, wherein the composition is within a hydroxypropylmethylcellulose (HPMC) capsule.
41. The method of any one of 38-40, wherein the solvent is triacetin, and the rheology modifier is isopropyl myristate (IPM).
42. The method of any one of 38-40, wherein the solvent is ethyl lactate, and the rheology modifier is isopropyl myristate (IPM).
43. The method of any one of 38-42, wherein the composition comprises a mineral particle.
44. The method of 43, wherein the mineral particle comprises silicon dioxide.
45. The method of any one of 38-42, wherein the composition comprises a viscosity enhancing agent.
46. The method of 45, wherein the viscosity enhancing agent comprises silicon dioxide, carnauba wax, or cetyl alcohol.
47. The method of any one of 38-46, wherein the pharmacologically active agent is selected from opioid, stimulant, and depressant.
48. The method of 47, wherein the pharmacologically active agent is an opioid.
49. The composition of 47, wherein the pharmacologically active agent is a mu opioid agonist.
50. The composition of 47, wherein the pharmacologically active agent is selected from oxycodone, oxymorphone, hydrocodone, and hydromorphone, either in the free base form or a pharmaceutically acceptable salt form thereof.
51. A composition comprising:

a pharmacologically active agent;

a solvent;

a network former; and

a mineral particle, wherein the mineral particle is present in the composition in an amount from about 1.9% by weight to about 3.0% by weight relative to the total weight of the composition.
52. The composition of 51, wherein the mineral particle comprises silicon dioxide, carnauba wax, or cetyl alcohol.
53. The composition of 51, wherein the pharmacologically active agent is selected from opioid, stimulant, and depressant.
54. The composition of 53, wherein the pharmacologically active agent is an opioid.
55. The composition of 53, wherein the pharmacologically active agent is a mu opioid agonist.
56. The composition of 53, wherein the pharmacologically active agent is selected from oxycodone, oxymorphone, hydrocodone, and hydromorphone, either in the free base form or a pharmaceutically acceptable salt form thereof.
57. The composition of any one of 51 to 56, wherein the solvent comprises triacetin.
58. The composition of any one of 51 to 57, wherein the solvent comprises ethyl lactate.
59. The composition of any one of 51 to 58, comprising about 15% by weight to about 45% by weight of the solvent relative to the total weight of the composition.

60. The composition of any one of 51 to 59, further comprising a rheology modifier.
61. The composition of 60, wherein the rheology modifier is IPM.
62. The composition of 61, comprising about 1% by weight to about 20% by weight of the IPM relative to the total weight of the composition.
63. The composition of any one of 51 to 62, wherein the composition comprises:

about 35% by weight to about 45% by weight of the HVLCM relative to the total weight of the composition,

about 15% by weight to about 45% by weight of the solvent relative to the total weight of the composition, and

about 4% by weight to about 5% by weight of the network former relative to the total weight of the composition.
64. The composition of any one of 51 to 63, wherein the HVLCM is SAIB, the solvent is triacetin, and the network former is CAB.
65. The composition of any one of 51 to 64, wherein the HVLCM is SAIB, the solvent is ethyl lactate, and the network former is CAB.
66. The composition of 64 or 65, comprising IPM.
67. The composition of any one of 51 to 64, wherein the pharmacologically active agent is present in the composition at about 2% by weight to about 50% by weight relative to the total weight of the composition.
68. The composition of any one of 51 to 64, wherein the composition is contained within a capsule.
69. A composition comprising:

an opioid;

triacetin or ethyl lactate;

isopropyl myristate (IPM); and

silicon dioxide, wherein the silicon dioxide, is present in the composition in an amount from about 1.9% by weight to about 3.0% by weight relative to the total weight of the composition.
70. The composition of 69, wherein the opioid is selected from oxycodone, oxymorphone, hydrocodone, and hydromorphone, either in the free base form or a pharmaceutically acceptable salt form thereof
71. The composition of 69, wherein the opioid is oxycodone.
72. The composition of any one of 69-71, wherein the opioid is present in the composition at about 5% by weight relative to the total weight of the composition.
73. A method for treating pain in a subject, the method comprising:

orally administering to the subject a composition comprising
an opioid;
a solvent;
a network former; and silicon dioxide, wherein the silicon dioxide is present in the composition in an amount from about 1.9% by weight to about 3.0% by weight relative to the total weight of the composition, wherein the composition is formulated for oral administration, and one or more symptoms or signs associated with the subject's pain is alleviated.
74. The method of 73, wherein the opioid is selected from oxycodone, oxymorphone, hydrocodone, and hydromorphone, either in the free base form or a pharmaceutically acceptable salt form thereof.
75. The method of 73, wherein the opioid is oxycodone.
76. The method of any one of 73-75, wherein the solvent comprises triacetin.
77. The method of any one of 73-76, wherein the solvent comprises ethyl lactate.
78. The method of any one of 73 to 77, wherein the composition comprises about 15% by weight to about 45% by weight of the solvent relative to the total weight of the composition.
79. The method of any one of 73 to 78, wherein the composition further comprises a rheology modifier.
80. The method of any one of 73-79, wherein the rheology modifier is IPM.
81. The method of any one of 73 to 80, wherein the pharmacologically active agent is present in the composition at about 2% by weight to about 50% by weight relative to the total weight of the composition.
82. The method of any one of 73 to 81, wherein the composition is contained within a capsule.
83. The method of any one of 73-82, wherein the composition is administered no more than twice in a 24-hour period.
84. A method for treating pain in a subject, the method comprising:

orally administering to the subject a composition comprising
an opioid;
triacetin or ethyl lactate;
isopropyl myristate (IPM); and
silicon dioxide, wherein the silicon dioxide, is present in the composition in an amount from about 1.9% by weight to about 3.0% by weight relative to the total weight of the composition, wherein the composition is formulated for oral administration, and one or more symptoms or signs associated with the subject's pain is alleviated.
85. The method of 84, wherein the opioid is selected from oxycodone, oxymorphone, hydrocodone, and hydromorphone, either in the free base form or a pharmaceutically acceptable salt form thereof.
86. The method of 84, wherein the opioid is oxycodone.
87. The method of any one of 84-86, wherein the opioid is present in the composition at about 5% by weight relative to the total weight of the composition.
88. The method of any one of 84-87, wherein the composition is encapsulated for oral administration.
89. The method of any one of 84 to 88, wherein the composition is contained within a capsule.
90. The method of any one of 84-89 wherein the composition is administered no more than twice in a 24-hour period.
91. A method of orally administering a composition, comprising:

improving reproducibility of an in vitro release profile of a composition by including about 1.9% by weight to about 3.0% by weight, relative to the total weight of the composition, of mineral particle in the composition, wherein the composition also includes a pharmacologically active agent, and a solvent; and

orally administering the composition.
92. A method of orally administering a composition, comprising:

decreasing the variability of an in vitro release profile of a composition by including about 1.9% by weight to about 3.0% by weight, relative to the total weight of the composition, of mineral particle in the composition, wherein the composition also includes a pharmacologically active agent, a solvent; and

orally administering the composition.
93. A method of orally administering an encapsulated composition, comprising:

forming a composition comprising:
a pharmacologically active agent,
a solvent, and
a mineral particle, wherein the mineral particle is present in the composition in an amount from about 1.9% by weight to about 3.0% by weight relative to the total weight of the composition;

improving an in vitro release profile of the composition by encapsulating the composition within a capsule comprising hydroxypropylmethylcellulose to form an encapsulated composition; and

orally administering the encapsulated composition.
94. A method of orally administering an encapsulated composition, comprising:

forming a composition comprising:
a pharmacologically active agent,
a solvent, and
a mineral particle, wherein the mineral particle is present in the composition in an amount from about 1.9% by weight to about 3.0% by weight relative to the total weight of the composition;

reducing exposure of the composition to water by encapsulating the composition within a capsule comprising hydroxypropylmethylcellulose to form an encapsulated composition; and

orally administering the encapsulated composition.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near one atmosphere. Standard abbreviations may be used, e.g., s or sec, second(s); min, minute(s); h or hr, hour(s); and the like.

### Example 1

### Preparation and Analysis of Extended Release Oxycodone Formulations

Formulations were prepared with the ingredients as set forth below in Table 1 below. The formulations were manually filled in Capsugel Licap size 00 (for 40 mg dose) and Qualicaps size 3 (for 10 mg) gelatin capsules, respectively.

The release rate of oxycodone base was determined from 2-4 capsules using a USP Apparatus 2 dissolution tester. Dissolution medium containing 750 ml 0.1N HCl was utilized for the first 2 hours, followed by the addition of 250 ml 0.2 M phosphate buffer to achieve a final pH of 6.8. The dissolution medium was maintained at 37° C. with 50 rpm paddle speed over the course of the 24 hour dissolution test. The capsules were placed in stainless steel (316SS) wire spiral capsule sinkers for dissolution testing. The standard sampling time points were 0.25, 0.5, 1, 2, 3, 6, 10, 12, 18 and 24 hours. A 1 mL sample was taken at each time point and assayed using reverse-phase HPLC at 240 nm wavelength. The HPLC parameters were as follows: Mobile phase A: 0.5% sodium dodecyl sulfate 1% glacial acetic acid, 20% acetonitrile; Mobile phase B: 100% acetonitrile. The mobile phase included: 65% Mobile phase A and 35% Mobile phase B; 240 nm wavelength.

The following protocol was used to screen the formulations for abuse resistance. Each capsule was soaked in 36 ml 0.1N HCl for 30 minutes, followed by the addition of 24 ml 200-proof ethanol to achieve a final 80-proof ethanol solution. The shaking speed was maintained at 240 rpm with incubation temperature at 25° C. The standard sampling time points were 0, 30 and 180 minutes post ethanol addition. A 1 mL sample was taken at each time point and assayed using reverse-phase HPLC at 240 nm wavelength. The mobile phase included 0.35% (w/v) SDS/0.7% (v/v) acetic acid/44% (v/v) acetonitrile in water.

The following protocol was used to analyze stability of the formulations. Dissolution performance was measured as described above following storage for 2 weeks at 25° C./60% RH and 40° C./75% RH. Dissolution testing was also performed following storage for 6 weeks under both of the above stability conditions.

TABLE 1

| | % w/w of component of each formulation | | | | | |
|---|---|---|---|---|---|---|
| | Formulation Identification | | | | | |
| Composition | Formulation 1 | Formulation 2 | Formulation 3 | Formulation 4 | Formulation 5 | Formulation 6 |
| Oxycodone free base | 5.13 | 5.13 | 5.13 | 5.13 | 5.13 | 5.13 |
| Sucrose acetate isobutyrate | 39.27 | 40.98 | 37.28 | 53.54 | 55.66 | 51.48 |
| (SAIB) | | | | | | |
| Triacetin (TA) | 26.18 | 27.32 | 24.85 | n/a | n/a | n/a |
| Ethyl lactate (EL) | n/a | n/a | n/a | 21.41 | 20.24 | 18.72 |
| Isopropyl Myristate (IPM) | n/a | 14.23 | n/a | 14.23 | 14.23 | 14.23 |
| Miglyol ® 812 | 14.23 | n/a | 18.97 | n/a | n/a | n/a |
| Cellulose acetate butyrate | 4.74 | 4.74 | 4.74 | 5.69 | 4.74 | 4.74 |
| (CAB) | | | | | | |
| Croscarmellose sodium | 8.54 | n/a | n/a | n/a | n/a | n/a |
| (Ac-Di-Sol ®) | | | | | | |
| Hydroxyethyl cellulose | n/a | 5.69 | 7.11 | n/a | n/a | n/a |
| (HEC) | | | | | | |
| Colloidal silicon dioxide (CSD) | 1.90 | 1.90 | 1.90 | n/a | n/a | n/a |
| Butylated hydroxytoluene | 0.02 | 0.02 | 0.02 | n/a | n/a | n/a |
| (BHT) | | | | | | |
| Low density- Hydroxy | n/a | n/a | n/a | n/a | n/a | 5.69 |
| propyl cellulose (L-HPC) | | | | | | |
| Capsule Shell | Gelatin | Gelatin | Gelatin | Gelatin | Gelatin | Gelatin |

The influence of dosage strength on release rate is shown in Table 2 below for 10 mg capsules and Table 3 below for 40 mg capsules.

TABLE 2
Dissolution Data - 10 mg capsules - initial

| | Dissolution | Dissolution Time Point/hrs | | | | | | | | | |
|---|---|---|---|---|---|---|---|---|---|---|---|
| Formulation | Rate Metric | 0.25 | 0.5 | 1 | 2 | 3 | 6 | 10 | 12 | 18 | 24 |
| Formulation | μ | 4.83 | 8.90 | 14.43 | 21.50 | 29.15 | 48.45 | 68.10 | 74.49 | 88.57 | 95.88 |
| 1 | σ | 0.28 | 0.52 | 1.19 | 1.77 | 4.31 | 9.30 | 10.63 | 10.88 | 8.45 | 4.51 |
| Formulation | μ | 1.74 | 4.73 | 10.25 | 19.49 | 26.99 | 40.19 | 50.41 | 54.45 | 64.28 | 70.76 |
| 2 | σ | 0.19 | 0.04 | 0.16 | 0.11 | 0.04 | 0.63 | 1.44 | 2.41 | 2.14 | 2.79 |
| Formulation | μ | 6.78 | 13.10 | 22.69 | 36.76 | 47.60 | 60.10 | 69.54 | 72.88 | 81.28 | 87.43 |
| 3 | σ | 0.71 | 0.45 | 0.49 | 0.71 | 0.93 | 0.88 | 0.22 | 0.39 | 0.92 | 0.76 |
| Formulation | μ | 6.87 | 12.39 | 18.00 | 24.70 | 28.58 | 38.13 | 46.39 | 49.37 | 57.35 | 63.66 |
| 4 | σ | 0.01 | 0.10 | 0.84 | 0.91 | 1.02 | 1.26 | 0.86 | 1.00 | 0.20 | 0.67 |
| Formulation | μ | 8.42 | 13.73 | 19.98 | 27.93 | 33.14 | 43.40 | 52.63 | 55.71 | 63.48 | 69.26 |
| 5 | σ | 1.99 | 3.22 | 4.91 | 7.51 | 8.81 | 10.95 | 12.72 | 13.34 | 12.93 | 13.10 |
| Formulation | μ | 7.02 | 12.01 | 19.33 | 28.48 | 33.80 | 42.50 | 50.14 | 53.24 | 60.83 | 66.71 |
| 6 | σ | 0.57 | 1.25 | 2.14 | 3.44 | 3.83 | 4.85 | 5.52 | 5.69 | 6.66 | 7.23 |

TABLE 3
Dissolution Data - 40 mg capsules - initial

| | Dissolution | Dissolution Time Point/hrs | | | | | | | | | |
|---|---|---|---|---|---|---|---|---|---|---|---|
| Formulation | Rate Metric | 0.25 | 0.5 | 1 | 2 | 3 | 6 | 10 | 12 | 18 | 24 |
| Formulation | μ | 3.66 | 6.94 | 11.93 | 19.01 | 23.99 | 34.54 | 46.59 | 51.56 | 64.40 | 73.94 |
| 1 | σ | 0.42 | 0.23 | 0.59 | 1.10 | 0.81 | 0.37 | 0.32 | 1.00 | 1.44 | 1.54 |
| Formulation | μ | 4.02 | 8.22 | 14.32 | 23.27 | 29.69 | 38.38 | 45.26 | 47.96 | 54.78 | 60.16 |
| 2 | σ | 0.75 | 1.53 | 2.08 | 2.89 | 1.87 | 1.72 | 1.30 | 1.17 | 0.81 | 0.49 |
| Formulation | μ | 3.59 | 7.36 | 13.11 | 21.77 | 28.06 | 36.79 | 44.05 | 46.87 | 54.35 | 60.58 |
| 3 | σ | 0.64 | 0.93 | 1.40 | 1.3 | 1.50 | 1.48 | 1.21 | 1.02 | 0.80 | 0.88 |
| Formulation | μ | 5.48 | 8.50 | 13.00 | 19.16 | 22.27 | 31.32 | 39.05 | 42.71 | 50.48 | 56.52 |
| 4 | σ | 0.25 | 0.35 | 0.05 | 0.37 | 0.28 | 0.40 | 0.37 | 1.12 | 0.47 | 0.07 |
| Formulation | μ | 4.15 | 7.35 | 11.09 | 16.35 | 18.99 | 26.23 | 33.17 | 36.26 | 42.44 | 47.83 |
| 5 | σ | 0.23 | 1.12 | 1.71 | 2.61 | 2.51 | 3.66 | 5.15 | 5.34 | 6.43 | 7.13 |
| Formulation | μ | 5.22 | 8.52 | 13.11 | 20.60 | 23.13 | 30.76 | 38.92 | 42.03 | 49.66 | 55.20 |
| 6 | σ | 0.36 | 0.40 | 0.44 | 0.27 | 0.12 | 0.41 | 0.97 | 0.86 | 0.25 | 0.47 |

Formulations were also tested for their abuse resistance properties. Results are shown in Table 4 below.

TABLE 4
Abuse Resistance Data.

| | | Extraction | | |
|---|---|---|---|---|
| | | Time Point/min* | | |
| | Dissolution Rate | 40 mg Capsules | | |
| Formulation | Metric | 0 | 30 | 180 |
| Formulation 1 | μ | 6.13 | 17.54 | 27.28 |
| | σ | 0.64 | 0.57 | 0.08 |
| Formulation 2 | μ | 7.39 | 23.81 | 37.82 |
| | σ | 2.43 | 8.34 | 11.19 |
| Formulation 3 | μ | 5.64 | 20.44 | 34.45 |
| | σ | 0.45 | 0.59 | 3.43 |
| Formulation 4 | μ | 4.99 | 12.59 | 27.96 |
| | σ | 0.04 | 0.80 | 5.30 |
| Formulation 5 | μ | 9.99 | 19.18 | 32.00 |
| | σ | 6.55 | 8.95 | 8.49 |
| Formulation 6 | μ | 6.41 | 16.85 | 34.19 |
| | σ | 1.08 | 0.97 | 1.93 |

Stability data for formulations 1, 2 and 3 is provided below in Tables Table 5A-5D below.

TABLE 5A
Dissolution Data 10 mg Capsules - 2 weeks (25° C./60% RH)

| | Dissolution | Dissolution Time Point/hrs | | | | | | | | | |
|---|---|---|---|---|---|---|---|---|---|---|---|
| Formulation | Rate Metric | 0.25 | 0.5 | 1 | 2 | 3 | 6 | 10 | 12 | 18 | 24 |
| Formulation | μ | 5.80 | 11.18 | 18.69 | 27.50 | 38.24 | 57.45 | 73.43 | 78.64 | 89.25 | 94.86 |
| 1 | σ | 0.01 | 0.35 | 0.29 | 2.78 | 10.71 | 19.68 | 19.69 | 18.69 | 12.04 | 6.75 |
| Formulation | μ | 7.22 | 13.37 | 22.45 | 34.43 | 41.54 | 53.04 | 61.88 | 65.11 | 73.72 | 80.90 |
| 2 | σ | 1.03 | 1.03 | 1.10 | 0.74 | 1.07 | 0.72 | 0.50 | 0.37 | 0.64 | 1.14 |
| Formulation | μ | 5.75 | 10.93 | 19.18 | 31.74 | 40.43 | 52.70 | 60.85 | 64.66 | 73.59 | 80.86 |
| 3 | σ | 0.34 | 0.59 | 0.81 | 1.71 | 1.20 | 2.83 | 0.04 | 0.04 | 1.87 | 3.15 |

TABLE 5B
Dissolution Data - 10 mg Capsules - 2 weeks (40° C./75% RH)

| | Dissolution | Dissolution Time Point/hrs | | | | | | | | | |
|---|---|---|---|---|---|---|---|---|---|---|---|
| Formulation | Rate Metric | 0.25 | 0.5 | 1 | 2 | 3 | 6 | 10 | 12 | 18 | 24 |
| Formulation | μ | 5.52 | 10.54 | 18.70 | 28.19 | 36.37 | 53.05 | 68.42 | 74.05 | 85.99 | 92.00 |
| 1 | σ | 0.27 | 0.18 | 0.10 | 2.62 | 5.42 | 8.63 | 10.51 | 10.75 | 9.21 | 6.48 |
| Formulation | μ | 8.19 | 14.38 | 23.06 | 34.85 | 43.50 | 54.86 | 63.54 | 67.13 | 75.12 | 80.71 |
| 2 | σ | 2.20 | 2.32 | 3.03 | 3.73 | 4.11 | 4.84 | 5.18 | 4.84 | 4.82 | 4.52 |
| Formulation | μ | 6.17 | 11.97 | 20.79 | 33.98 | 44.55 | 58.77 | 68.84 | 73.33 | 83.31 | 91.29 |
| 3 | σ | 1.44 | 2.98 | 5.56 | 8.89 | 11.73 | 16.21 | 18.78 | 20.37 | 23.42 | 25.80 |

TABLE 5C
Dissolution Data - 10 mg Capsules - 6 weeks (25° C./60% RH)

| | Dissolution | Dissolution Time Point/hrs | | | | | | | | | |
|---|---|---|---|---|---|---|---|---|---|---|---|
| Formulation | Rate Metric | 0.25 | 0.5 | 1 | 2 | 3 | 6 | 10 | 12 | 18 | 24 |
| Formulation | μ | 6.80 | 13.62 | 22.61 | 34.11 | 43.42 | 63.10 | 79.09 | 85.19 | 96.18 | 101.23 |
| 1 | σ | 0.21 | 0.78 | 1.51 | 0.51 | 1.14 | 1.99 | 1.67 | 1.30 | 0.60 | 2.09 |
| Formulation | μ | 7.95 | 13.89 | 23.06 | 35.59 | 43.76 | 56.19 | 65.66 | 69.36 | 77.74 | 84.21 |
| 2 | σ | 0.78 | 1.56 | 3.21 | 4.82 | 5.44 | 6.97 | 6.12 | 6.64 | 6.20 | 5.60 |
| Formulation | μ | 6.43 | 11.28 | 19.30 | 30.32 | 36.93 | 48.53 | 56.47 | 59.43 | 65.87 | 73.82 |
| 3 | σ | 1.76 | 2.51 | 3.97 | 5.30 | 5.30 | 6.63 | 7.58 | 7.71 | 10.10 | 9.86 |

TABLE 5D
Dissolution Data - 10 mg Capsules - 6 weeks (40° C./75% RH)

| | Dissolution | Dissolution Time Point/hrs | | | | | | | | | |
|---|---|---|---|---|---|---|---|---|---|---|---|
| Formulation | Rate Metric | 0.25 | 0.5 | 1 | 2 | 3 | 6 | 10 | 12 | 18 | 24 |
| Formulation | μ | 4.57 | 9.93 | 18.48 | 35.35 | 48.25 | 69.17 | 84.27 | 88.77 | 95.47 | 97.58 |
| 1 | σ | 0.15 | 0.15 | 1.50 | 3.43 | 3.87 | 5.13 | 3.85 | 2.86 | 0.17 | 0.35 |
| Formulation | μ | 7.74 | 13.52 | 21.14 | 31.97 | 39.44 | 52.07 | 60.48 | 63.82 | 71.09 | 77.23 |
| 2 | σ | 1.17 | 1.67 | 2.30 | 2.86 | 3.01 | 3.70 | 4.01 | 4.12 | 5.25 | 5.14 |
| Formulation | μ | 5.39 | 9.70 | 17.03 | 27.73 | 35.35 | 47.93 | 57.79 | 61.36 | 70.57 | 77.72 |
| 3 | σ | 0.80 | 0.59 | 1.41 | 1.91 | 2.04 | 1.83 | 1.94 | 2.14 | 1.17 | 0.44 |

### Example 3

### PK Analysis of Extended Release Oxycodone Formulations

### Materials and Methods

This study was an open-label, single-dose, randomized crossover study to evaluate the pharmacokinetics and relative bioavailability of oxycodone following oral administration of 40 mg doses. This study was designed to evaluate the PK and bioavailability of single oral 40 mg doses of modified formulations of oxycodone (Formulations 1, 2, and 3).

This was a randomized, open-label, crossover study in healthy volunteers. Eighteen (18) subjects aged 18-55 years who met inclusion and exclusion criteria were enrolled. Three test modified oxycodone formulations (i.e., Formulations 1, 2 and 3) were evaluated under fed conditions.

Results

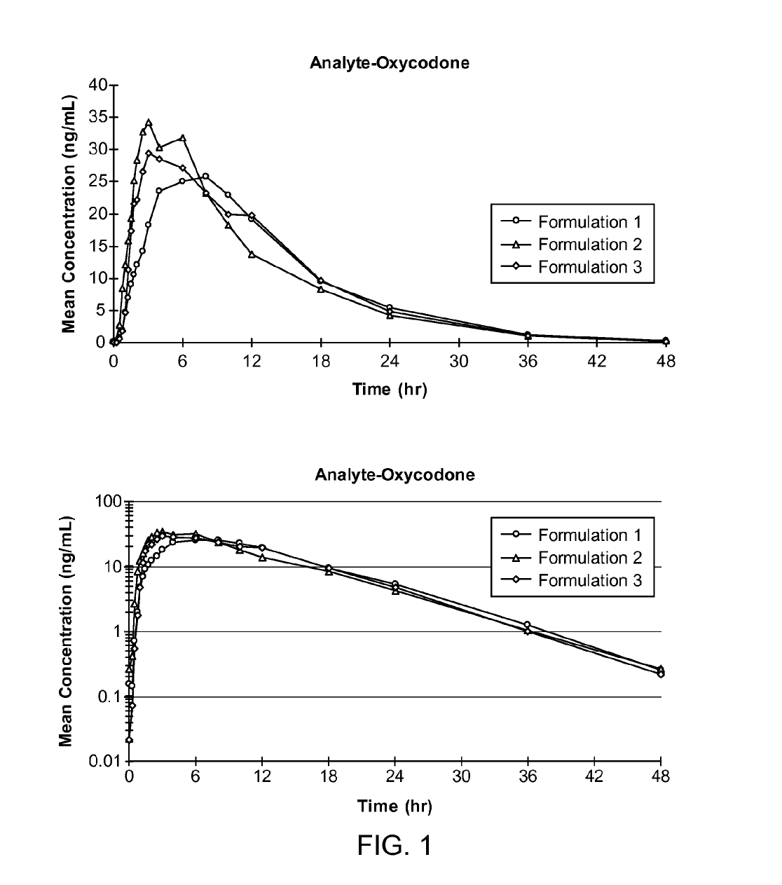

The mean plasma oxycodone concentration profiles for oxycodone PK parameters following single oral doses of each formulation tested in the study are shown in FIG. 1. PK parameters of oxycodone after administration of Formulation 1, Formulation 2, and Formulation 3 are shown below in Table 6.

TABLE 6

| | Formulation 1 | | | | Formulation 2 | | | |
|---|---|---|---|---|---|---|---|---|
| Parameter | n | Mean | SD | CV % | n | Mean | SD | CV % |
| Tmax (hr) | 12 | 5.99 | 3.22 | 53.76 | 12 | 3.06 | 1.45 | 47.57 |
| Cmax (ng/mL) | 12 | 33.6 | 10.4 | 30.87 | 12 | 45.9 | 22.1 | 48.08 |
| AUC₀₋₂₄ (hr* | 12 | 366.0 | 117.1 | 31.99 | 12 | 377.6 | 154.2 | 40.82 |
| ng/mL) | | | | | | | | |
| AUC₀₋₃₆ (hr* | 12 | 405.8 | 140.6 | 34.64 | 11 | 430.4 | 154.4 | 35.88 |
| ng/mL) | | | | | | | | |
| AUC₀₋₄₈ (hr* | 12 | 415.2 | 147.4 | 35.51 | 11 | 439.2 | 155.1 | 35.30 |
| ng/mL) | | | | | | | | |
| AUCₗₐₛₜ (hr* | 12 | 412.4 | 149.1 | 36.17 | 12 | 413.5 | 166.3 | 40.22 |
| ng/mL) | | | | | | | | |
| AUC_{inf} (hr* | 12 | 418.5 | 149.9 | 35.81 | 11 | 443.7 | 154.0 | 34.72 |
| ng/mL) | | | | | | | | |
| AUC_{Extrap} (%) | 12 | 1.61 | 0.82 | 51.15 | 11 | 2.03 | 1.72 | 84.69 |
| λ_{z} (1/hr) | 12 | 0.1197 | 0.0238 | 19.90 | 11 | 0.1113 | 0.0290 | 26.09 |
| T_{1/2} (1/hr) | 12 | 6.07 | 1.56 | 25.66 | 11 | 6.76 | 2.35 | 34.70 |
| Tₗₐₛₜ (1/hr) | 12 | 39.97 | 7.78 | 19.47 | 12 | 38.98 | 9.02 | 23.15 |
| Cₗₐₛₜ (ng/mL) | 12 | 0.694 | 0.224 | 32.34 | 12 | 0.827 | 0.411 | 49.74 |

| | Formulation 3 | | | |
|---|---|---|---|---|
| Parameter | n | Mean | SD | CV % |
| Tmax (hr) | 12 | 4.78 | 2.88 | 60.26 |
| Cmax (ng/mL) | 12 | 38.9 | 19.0 | 48.87 |
| AUC₀₋₂₄ (hr* ng/mL) | 12 | 392.4 | 114.5 | 29.18 |
| AUC₀₋₃₆ (hr* ng/mL) | 11 | 432.4 | 139.7 | 32.30 |
| AUC₀₋₄₈ (hr* ng/mL) | 11 | 440.4 | 145.8 | 33.11 |
| AUCₗₐₛₜ (hr* ng/mL) | 12 | 431.5 | 142.0 | 32.90 |
| AUC_{inf} (hr* ng/mL) | 11 | 442.9 | 147.7 | 33.35 |
| AUC_{Extrap} (%) | 11 | 1.08 | 0.80 | 73.97 |
| λ_{z} (1/hr) | 11 | 0.1309 | 0.0199 | 15.21 |
| T_{1/2} (1/hr) | 11 | 5.41 | 0.83 | 15.40 |
| Tₗₐₛₜ (1/hr) | 12 | 38.98 | 9.02 | 23.14 |
| Cₗₐₛₜ (ng/mL) | 12 | 0.726 | 0.638 | 87.91 |

### Example 4

### Preparation and Analysis of Extended Release Oxycodone Compositions (Reference Formulation A and Formulations 7-10)

Additional compositions (Formulations 7-10) with varying concentrations of isopropyl myristate (IPM) and silicon dioxide (SiO₂) were prepared and compared with Reference Formulation A (with BHT) to determine the effect of these components on inter-capsule dissolution variability and rheology as indicated below.

### Materials and Methods

The compositions were prepared as follows to provide the compositions indicated in Table 7 (below). Sucrose Acetate Isobutyrate (SAIB) was transferred into a Ross mixer at an elevated temperature (50° C.) and dissolved in triacetin (TA) and isopropyl myristate (IPM) and uniformly mixed. When present in the composition, butylated hydroxytoluene (BHT) was added prior to uniformly mixing with TA and IPM. Colloidal silicon dioxide (CSD) particles were added into the SAIB solution in the Ross mixer and were dispersed uniformly. Cellulose acetate butyrate (CAB) particles were sieved and fed into the Ross mixer and dispersed and dissolved in the content of the mixer at the elevated temperature. The oxycodone particles were introduced into the Ross mixer and dispersed in the content of the mixer, keeping the same process temperature. Hydroxyethyl cellulose (HEC) was then added into the Ross mixer and dispersed. In order to assure complete dispersion of all particles (oxycodone, SiO₂, HEC), high shear mixers (dispenser and emulsifier) may be used for pre-set time periods after the introduction of these solid particles into the Ross mixer.

For the capsule filling operation, the compositions were transferred from the Ross mixer via a temperature controlled (or insulated) (at 50-60° C.) pump and hoses to the capsule filling equipment. The temperature of the compositions was maintained at 50-60° C. during the capsule filling operations.

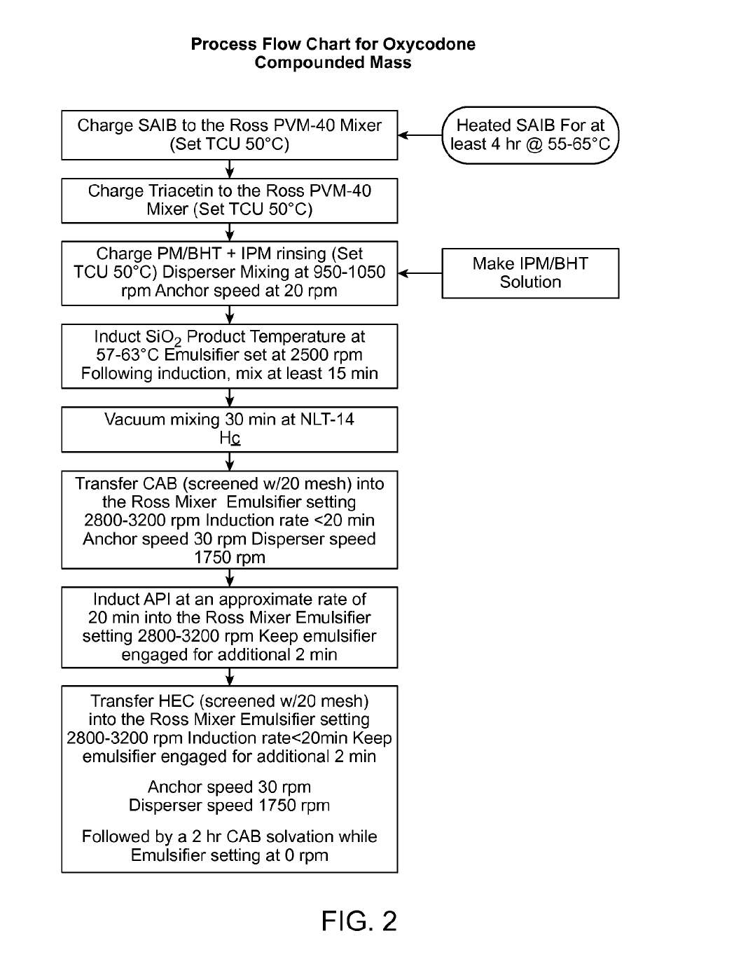

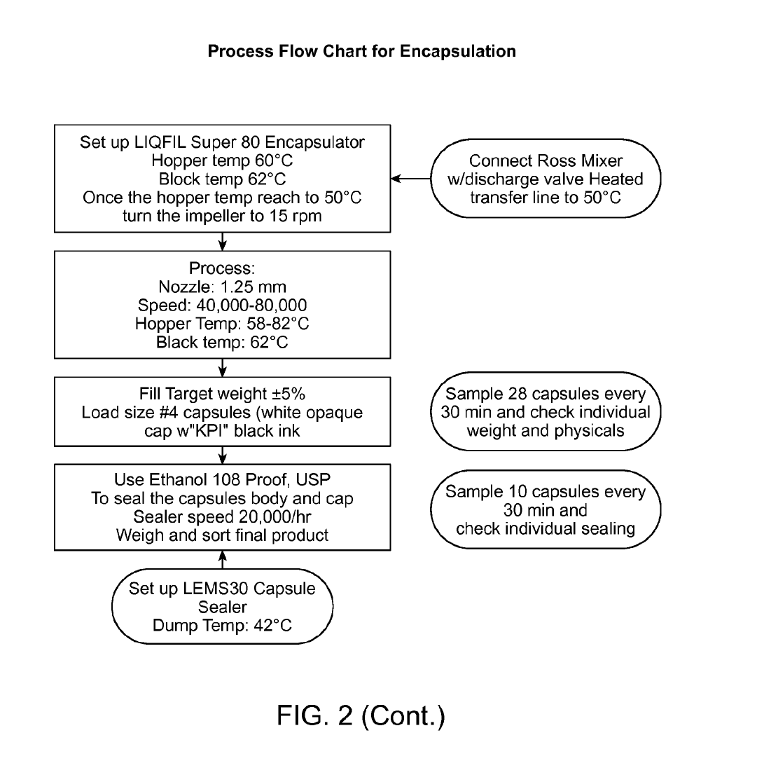

Individual compositions were encapsulated in size 4 (5 mg dose) or size 00 (40 mg dose) gelatin capsules. Encapsulation was achieved using a Capsugel CFS 1000™ apparatus. It was observed that increasing the temperature of the composition and the filling pump, e.g., from about 60° C. to about 75° C., reduced the stringiness of the composition, thereby facilitating the separation of the composition from the nozzle into the capsule shell and allowing clean movement to the next capsule station. The reduced stringiness of the composition also allowed the motor speed setting (fill rate) to be increased, e.g., to a motor speed set point range of about 50% to about 60% (500-600 capsules per hour). Size 00 capsules were successfully filled using, e.g., a 1.8 mm filling nozzle. Size 4 capsules were successfully filled using, e.g., a 2.0-2.2 mm nozzle. An exemplary composition preparation and encapsulation method is depicted graphically in FIG. 2.

TABLE 7

| | Vehicle Composition (% w/w) | | | | | | | (mg) |
|---|---|---|---|---|---|---|---|---|
| ID | SAIB | TA | IPM | CAB | HEC | SiO₂ | BHT | Oxycodone |
| Reference | 43 | 29 | 15 | 5 | 6 | 2 | 0.02 | 40 |
| Formulation A | | | | | | | | |
| Formulation 7 | 47 | 32 | 8 | 5 | 6 | 2 | 0.02 | 40 |
| Formulation 8 | 52 | 35 | 0 | 5 | 6 | 2 | 0.02 | 40 |
| Formulation 9 | 44 | 29 | 15 | 5 | 6 | 1 | 0.02 | 40 |
| Formulation 10 | 44 | 30 | 15 | 5 | 6 | 0 | 0.02 | 40 |

Dissolution Testing

Four capsules from each composition were tested with USP Apparatus 2 to evaluate the effect on inter-capsule dissolution variability. The release rate of oxycodone base was determined using a USP Apparatus 2 dissolution tester. Dissolution medium containing 1000 ml 0.1 N HCl with 0.5% (w/w) SDS was maintained at 37° C. over the course of the 24 hour dissolution test. A 20 mesh screen hanging basket was incorporated to hold the test article and the paddle speed was set to 100 rpm. The standard sampling time points were 0.5, 2, 3, 6, 12, 18 and 24 hours. A 1 mL sample was taken at each time point and assayed using reverse-phase HPLC at 240 nm wavelength. The mobile phase included 0.35% (w/v) SDS/0.7% (v/v) acetic acid/44% (v/v) acetonitrile in water.

Rheology Testing

Samples of the above compositions (Table 7) were analyzed for rheological properties using an Anton Paar MCR301 Rheometer. The samples were exposed to increasing dynamic strain (0.1 to 100%) at a constant angular frequency (10 s⁻¹) at 25° C.

### Results

Dissolution Testing Results

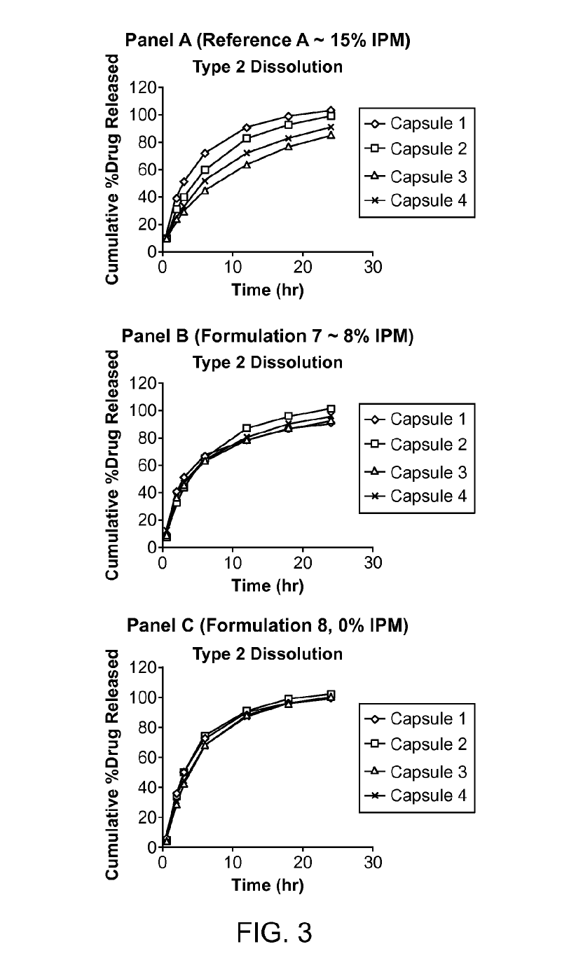

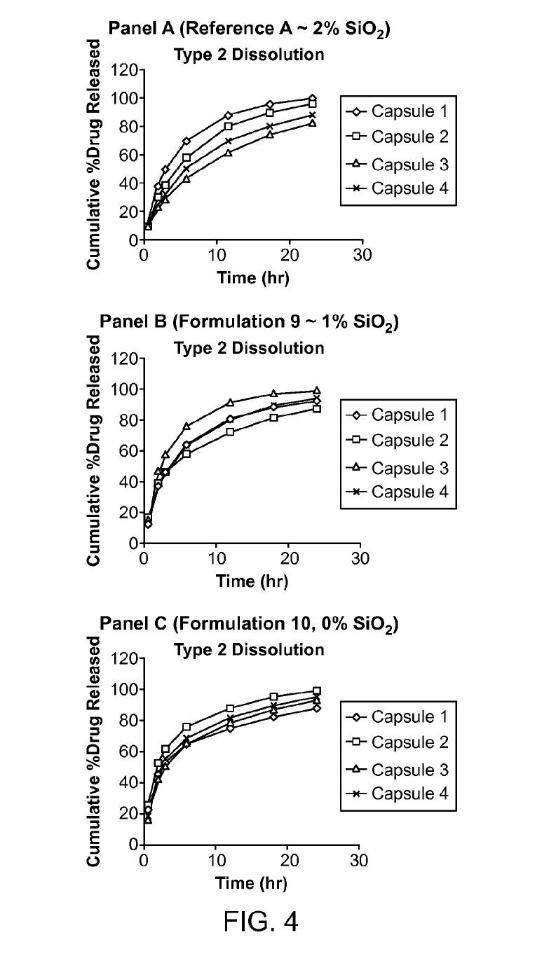

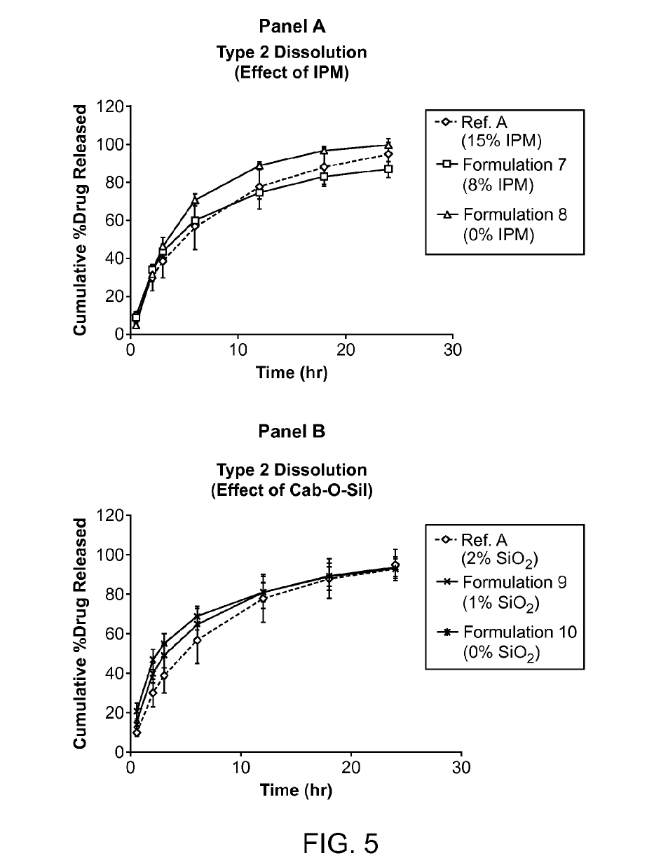

The results of the dissolution experiments are shown in FIGS. 3 and 4. The in vitro dissolution results showed a reduction in the inter-capsule dissolution variability with a reduction in the concentration of IPM in the composition (see FIG. 3, Panels A-C). Sample variability was significant when the level of SiO₂ in the composition was less than 2% as shown in FIG. 4, Panels A-C. The effects of adjusting the concentration of IPM and SiO₂ on the dissolution profiles of the compositions are shown in FIG. 5, Panels A and B, respectively, wherein the 0% IPM composition exhibited increased mean release at later time points, and the 0% SiO₂ composition exhibited increased mean release at earlier time points.

Rheology Testing Results

Table 8 (below) summarizes the viscoelastic outputs at the linear viscoelastic range for the rheology analysis.

TABLE 8

| | | | Storage | Loss | |
|---|---|---|---|---|---|
| | | Complex | Modulus | Modulus | Damping |
| | | Viscosity | (G′) | (G″) | Factor |
| ID | Description | (Pa · s) | (Pa) | (Pa) | (G″/G′) |
| Reference | (15% IPM, | 53.3 | 239 | 476 | 1.99 |
| Form- | 2% SiO₂) | | | | |
| ulation A | | | | | |
| Form- | (8% IPM) | 90.3 | 473 | 769 | 1.63 |
| ulation 7 | | | | | |
| Form- | (0% IPM) | 158 | 993 | 1230 | 1.24 |
| ulation 8 | | | | | |
| Form- | (1% SiO₂) | 51.8 | 229 | 464 | 2.02 |
| ulation 9 | | | | | |
| Form- | (0% SiO₂) | 41.1 | 173 | 373 | 2.16 |
| ulation 10 | | | | | |

Compositions with lower % IPM (as compared to Reference Formulation A) had higher complex viscosity and higher elastic property (higher G′ and lower G″/G′). Without intending to be bound by any particular theory, these properties may have resulted in the observed decrease in inter-capsule dissolution variability. Compositions with lower concentrations of SiO₂ had lower viscosity and lower elastic property (lower G′ and high G″/G′) similar to Reference Formulation A. Without intending to be bound by any particular theory, the lower elastic property could relate to an increase in the deformation of the composition structure due to hydrodynamic forces in the dissolution media.

### Example 5

### Preparation and Analysis of Extended Release Oxycodone Compositions (Formulations 11 and 12)

Additional compositions (Formulations 11 and 12) and Formulation A′ (Reference Formulation A without BHT in HPMC capsule) were prepared and characterized with respect to inter-capsule dissolution variability, rheology and abuse deterrence characteristics as indicated below.

### Materials and Methods

The compositions were prepared to provide the compositions indicated in Table 9 (below). Composition components were blended and individual compositions were encapsulated as described above for Example 4, with the exception that HPMC capsules were used in place of gelatin capsules.

TABLE 9

| Composition | | | |
|---|---|---|---|
| (% w/w) | Formulation A′ | Formulation 11 | Formulation 12 |
| SAIB | 40.99 | 40.42 | 39.85 |
| TA | 27.32 | 26.94 | 26.56 |
| IPM | 14.23 | 14.23 | 14.23 |
| CAB | 4.74 | 4.74 | 4.74 |
| HEC | 5.69 | 5.69 | 5.69 |
| Colloidal SiO₂ | 1.90 | 2.85 | 3.79 |
| Micronized | 5.13 | 5.13 | 5.13 |
| oxycodone base | | | |
| Capsule shell | HPMC | HPMC | HPMC |

Dissolution Testing

Six capsules from each composition lot were tested according to the testing conditions discussed above to evaluate the effect on mean release and inter-capsule dissolution variability.

Rheology Testing

Triplicate samples for each composition were subjected to rheology testing as discussed above.

Abuse Deterrence

Four capsules from each composition were tested for abuse deterrence characteristics. The release rate of oxycodone base was determined using an isocratic HPLC method at defined time points. The capsules were subjected to 60 mL of acidified 80-proof ethanol with vigorous shaking Each capsule was placed in a wide mouth round jar containing 36 mL of 0.1 N HCl and 24 mL 200-proof of ethanol. The sample jar was placed in a shaking incubator maintained at 25° C. with 240 rpm shaking speed over the course of the 3 hour extraction test. The sampling time points were 0.5, 1, and 3 hours. A 1 mL sample was taken at each time point and assayed using reverse-phase HPLC at 240 nm wavelength. The mobile phase included 0.35% (w/v) SDS/0.7% (v/v) acetic acid/44% (v/v) acetonitrile in water.

### Results

Dissolution Testing Results

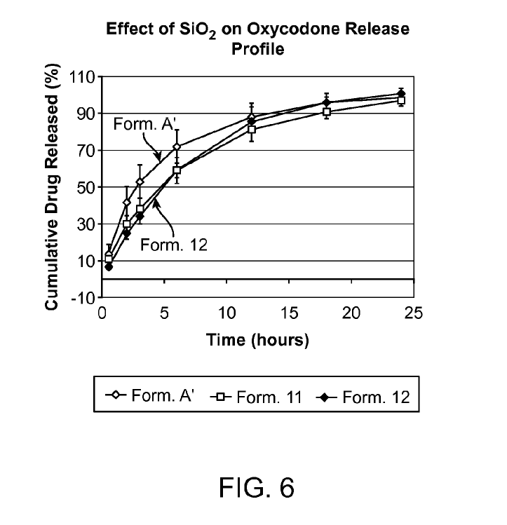

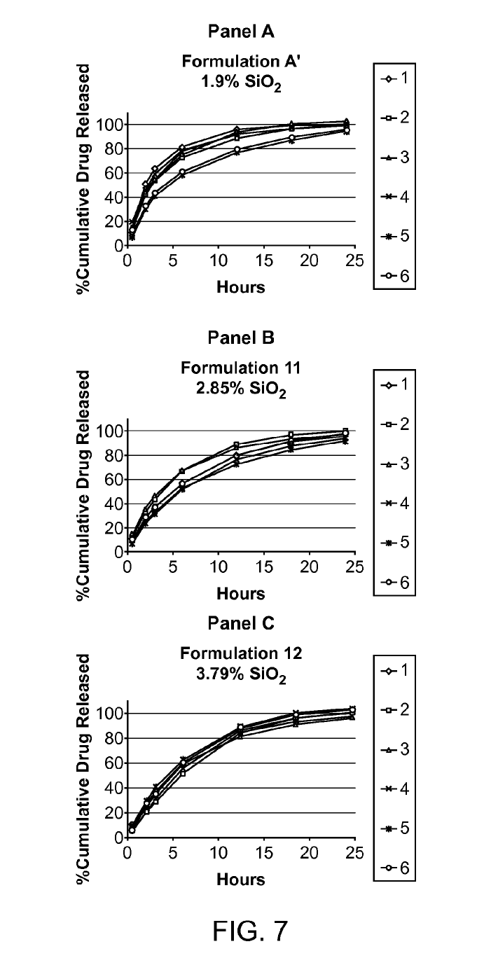

The results of the dissolution experiments are provided in FIG. 6; FIG. 7, Panels A-C; and Table 10 (below). The results demonstrate a) a reduction in the mean release prior to 12 hours with increasing SiO₂ concentration as shown in FIG. 6, and b) a reduction in the inter-capsule dissolution variability with increasing SiO₂ concentration as shown in FIG. 7, Panels A-C, and Table 10.

TABLE 10

| | SiO₂ | Sam- | Time | | | | | | | | |
|---|---|---|---|---|---|---|---|---|---|---|---|
| | (% | ple | Point | | | | | | | | |
| ID | w/w) | No. | (hrs.) | 0.5 | 2 | 3 | 6 | 12 | 18 | 24 | Sp* |
| Formula- | 1.90 | 6 | Mean | 14 | 42 | 53 | 72 | 88 | 96 | 99 | 7 |
| tion A′ | | | SD | 5 | 8 | 9 | 9 | 8 | 5 | 3 | |
| Formula- | 2.85 | 6 | Mean | 11 | 30 | 38 | 59 | 81 | 91 | 97 | 5 |
| tion 11 | | | SD | 3 | 5 | 6 | 7 | 6 | 4 | 3 | |
| Formula- | 3.79 | 6 | Mean | 7 | 25 | 34 | 59 | 86 | 96 | 101 | 3 |
| tion 12 | | | SD | 1 | 3 | 4 | 4 | 3 | 3 | 3 | |

*Sp as used herein = Pooled standard deviation which is calculated as provided below:
${s}_{p} = {( \frac{( {n}_{1} - 1 ) {s}_{1}^{2} + ( {n}_{2} - 1 ) {s}_{2}^{2} + … ( {n}_{k} - 1 ) {s}_{k}^{2}}{{n}_{1} + {n}_{2} + … {n}_{k} - k} )}^{1 \text{/} 2}$ wherein, n = sample number and the suffixes 1, 2, . . . k refer to the different series of measurements.

Rheology Testing Results

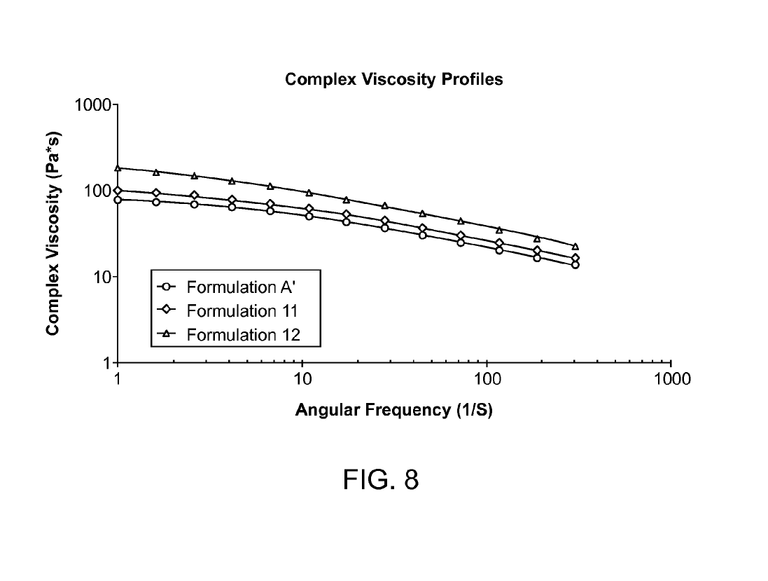

Table 11 (below) summarizes the results measured at angular frequency of 10 s⁻¹. Complex viscosity profiles with angular frequency sweep are shown in FIG. 8.

TABLE 11

| | | | | | | Storage | Loss | |
|---|---|---|---|---|---|---|---|---|
| | | | | | Complex | Modulus | Modulus | Damping |
| | SAIB | TA | IPM | SiO₂ | Viscosity | (Pa) | (Pa) | Factor |
| ID | (% w/w) | (% w/w) | (% w/w) | (% w/w) | (Pa · s) | (G′) | (G″) | (G″/G′) |
| Formulation | 40.98 | 27.32 | 14.23 | 1.90 | 49.53 | 245.00 | 474.33 | 1.93 |
| A′ | | | | | | | | |
| Formulation | 40.41 | 26.64 | 14.23 | 2.85 | 61.63 | 311.00 | 586.67 | 1.89 |
| 11 | | | | | | | | |
| Formulation | 39.85 | 26.56 | 14.23 | 3.79 | 95.50 | 540.67 | 874.67 | 1.62 |
| 12 | | | | | | | | |

As shown, increasing SiO₂ concentration above about 2% increases complex viscosity which may lead to decreasing matrix deformation and therefore low inter-capsule variability during dissolution testing. In addition to increase of the Loss Modulus, it is surprising that the extent of increase of Storage Modulus (G′) is even higher which results in lower damping factor (G″/G′) for Formulations 11 and 12 as compared with Formulation A′ (Reference Formulation A without BHT in HPMC capsule). In other words, increasing the amount of SiO₂ not only increases viscosity but also increases elasticity. Without intending to be bound by any particular theory, a lower damping factor may indicate a more stable microstructure which may lead to more stable dissolution stability.

Abuse Deterrence Results

The % of oxycodone released from each composition at sampling time points 0.5, 1, and 3 hours as determined by reverse-phase HPLC is provided in Table 12 below.

TABLE 12

| | SiO₂ | Sample | Time point | | | |
|---|---|---|---|---|---|---|
| ID | (%) | # | (hrs) | 0.5 | 1 | 3 |
| Formulation | 1.9 | 4 | Mean | 22 | 29 | 46 |
| A′ | | | SD | 3 | 3 | 5 |
| Formulation | 2.85 | 4 | Mean | 18 | 25 | 41 |
| 11 | | | SD | 3 | 4 | 5 |
| Formulation | 3.79 | 4 | Mean | 17 | 23 | 36 |
| 12 | | | SD | 3 | 4 | 7 |

As shown above, the % release of oxycodone decreased at each time point with increased SiO₂ concentration, suggesting an improvement in this abuse deterrence characteristic with increased SiO₂ in the tested range.

### Example 6

### One Month Stability Analysis of Extended Release Oxycodone Compositions (Formulations 11 and 12)

### Materials and Methods

Formulation A′ (Reference Formulation A without BHT in HPMC capsule) and Formulations 11 and 12 were stored at 25° C./60% RH or 40° C./75% RH for a one-month period of time. Six capsules from each composition lot were tested according to the testing conditions discussed above to evaluate the effect on mean release and inter-capsule dissolution variability.

### Results

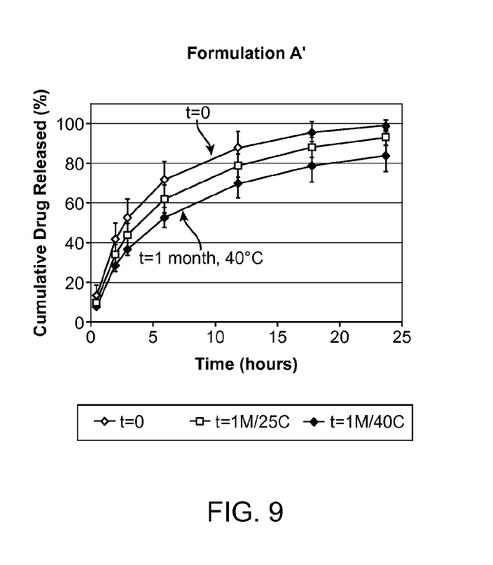

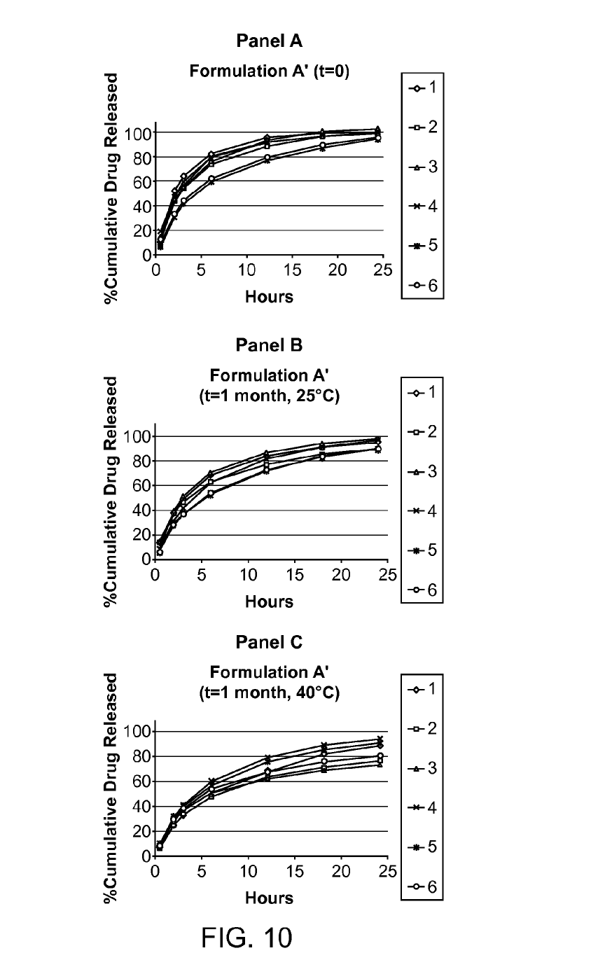

The results for Formulation A′ are provided in FIG. 9; FIG. 10, Panels A-C; and Table 13 below. Mean release is decreased for the stored Formulation A′ capsule samples relative to the T=0 samples as shown in FIG. 9. Inter-capsule variation was similar for the stored Formulation A′ samples and the T=0 samples as shown in FIG. 10, Panels A-C and Table 13.

TABLE 13

| | | Time | | | Time | | | | | | | | |
|---|---|---|---|---|---|---|---|---|---|---|---|---|---|
| | SiO₂ | Point | Storage | Sample | Point | | | | | | | | |
| ID | (%) | (Months) | Condition | No. | (hrs) | 0.5 | 2 | 3 | 6 | 12 | 18 | 24 | Sp |
| Formulation | 1.90 | 0 | NA | 6 | Mean | 14 | 42 | 53 | 72 | 88 | 96 | 99 | 7 |
| A′ | | | | | SD | 5 | 8 | 9 | 9 | 8 | 5 | 3 | |
| | | 1 | 25° C./60% RH | 6 | Mean | 10 | 34 | 44 | 62 | 79 | 88 | 93 | 5 |
| | | | | | SD | 3 | 5 | 6 | 7 | 6 | 5 | 4 | |
| | | | 40° C./75% RH | 6 | Mean | 8 | 29 | 37 | 53 | 70 | 79 | 84 | 6 |
| | | | | | SD | 1 | 3 | 3 | 5 | 7 | 8 | 8 | |

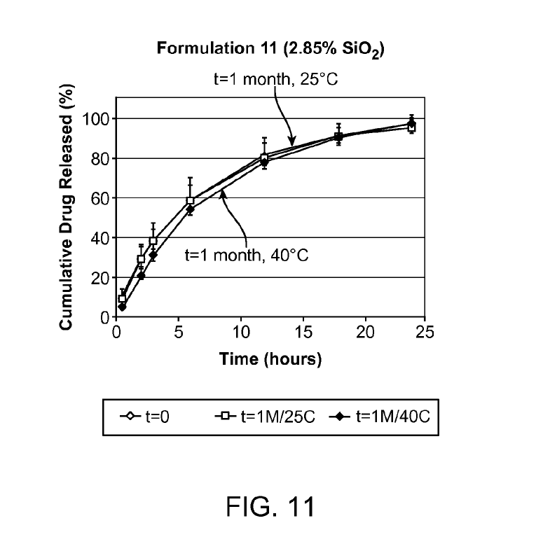

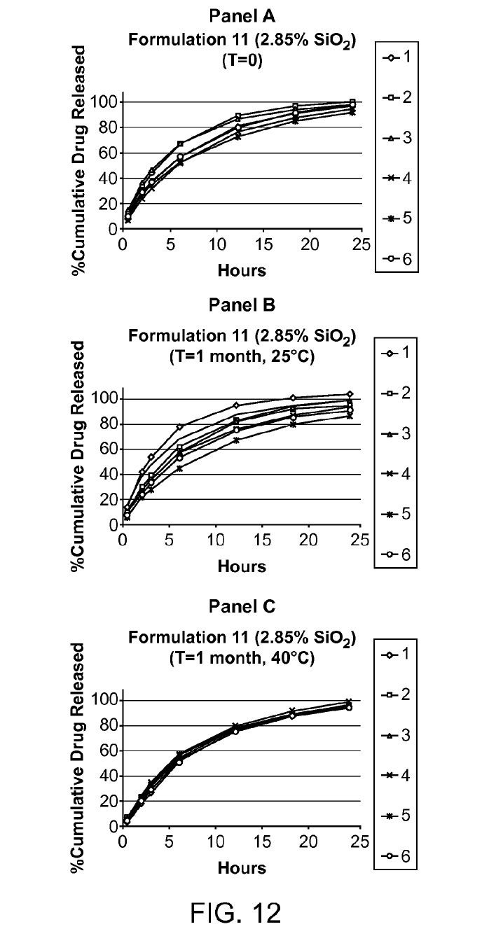

The results for Formulation 11 are provided in FIG. 11; FIG. 12, Panels A-C; and Table 14 below. Mean release is not significantly changed for the Formulation 11 samples relative to the T=0 samples as shown in FIG. 11. Sample variation was decreased for the Formulation 11 samples stored at 40° C./75% RH relative to the T=0 samples as shown in FIG. 12, Panels A-C and Table 14.

TABLE 14

| | | Time | | | Time | | | | | | | | |
|---|---|---|---|---|---|---|---|---|---|---|---|---|---|
| | SiO₂ | Point | Storage | Sample | Point | | | | | | | | |
| ID | (%) | (Months) | Condition | No. | (hrs) | 0.5 | 2 | 3 | 6 | 12 | 18 | 24 | Sp |
| Formulation | 2.85 | 0 | NA | 6 | Mean | 11 | 30 | 38 | 59 | 81 | 91 | 97 | 5 |
| 11 | | | | | SD | 3 | 5 | 6 | 7 | 6 | 4 | 3 | |
| | | 1 | 25° C./60% RH | 6 | Mean | 9 | 29 | 38 | 59 | 80 | 90 | 95 | 8 |
| | | | | | SD | 3 | 7 | 9 | 11 | 10 | 7 | 6 | |
| | | | 40° C./75% RH | 6 | Mean | 5 | 21 | 31 | 54 | 78 | 90 | 97 | 2 |
| | | | | | SD | 1 | 2 | 3 | 3 | 2 | 2 | 2 | |

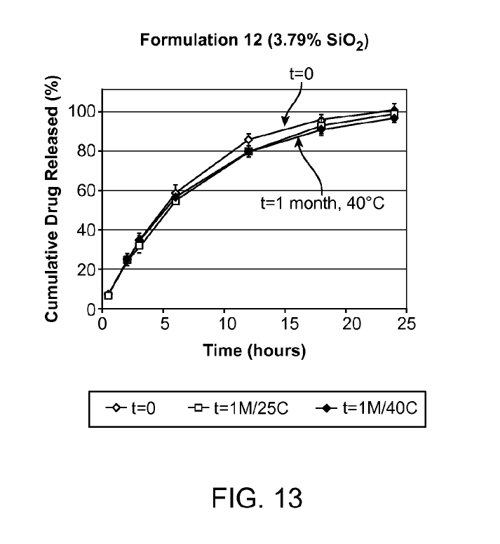

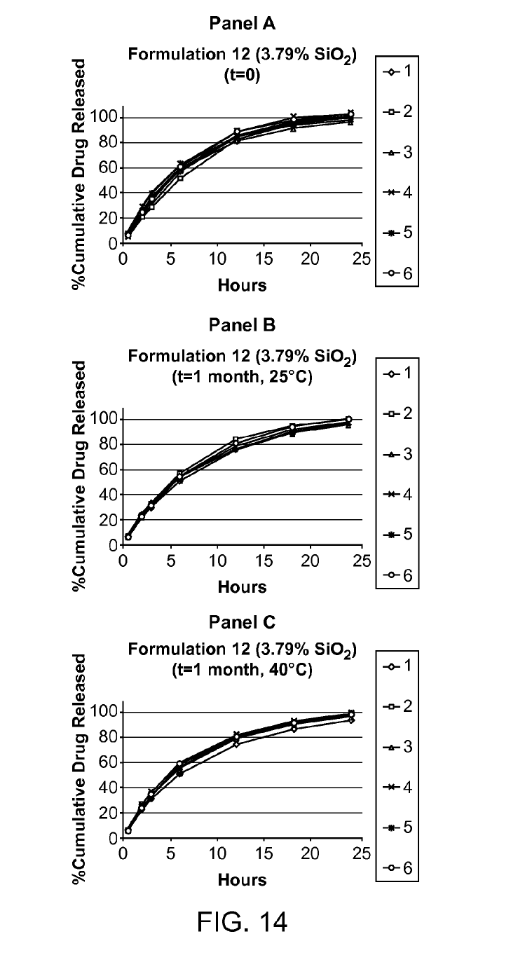

The results for Formulation 12 are provided in FIG. 13; FIG. 14, Panels A-C; and Table 15 below. Mean release is not significantly changed for the Formulation 12 relative to the T=0 samples as shown in FIG. 13. Sample variation was low and similar for the Formulation 12 samples stored at 25° C./60% RH and 40° C./75% RH relative to the T=0 samples as shown in FIG. 14, Panels A-C and Table 15.

TABLE 15

| | | Time | | | Time | | | | | | | | |
|---|---|---|---|---|---|---|---|---|---|---|---|---|---|
| | SiO₂ | Point | Storage | Sample | Point | | | | | | | | |
| ID | (%) | (Months) | Condition | No. | (hrs) | 0.5 | 2 | 3 | 6 | 12 | 18 | 24 | Sp |
| Formulation | 3.79 | 0 | NA | 6 | Mean | 7 | 25 | 34 | 59 | 86 | 96 | 101 | 3 |
| 12 | | | | | SD | 1 | 3 | 4 | 4 | 3 | 3 | 3 | |
| | | 1 | 25° C./60% RH | 6 | Mean | 7 | 24 | 32 | 55 | 80 | 93 | 99 | 2 |
| | | | | | SD | 0 | 1 | 1 | 2 | 3 | 2 | 2 | |
| | | | 40° C./75% RH | 6 | Mean | 7 | 25 | 35 | 57 | 80 | 91 | 97 | 2 |
| | | | | | SD | 1 | 2 | 2 | 3 | 2 | 2 | 2 | |

### Example 7

### Preparation and Analysis of Extended Release Hydromorphone HCl Compositions (Formulations 13-24)

Hydromorphone compositions were prepared and characterized with respect to dissolution profile, inter-capsule dissolution variability, and abuse deterrence characteristics as indicated below.

### Materials and Methods

The compositions were prepared to provide the compositions indicated in Table 16 (below). Composition component amounts are % w/w relative to the total weight of the formulation including hydromorphone HCl prior to encapsulation unless otherwise noted.

The formulations were prepared in 100 g scale. The temperature of the formulation compounding was maintained at 80° C.±5° C. and the mixing speed was maintained at 1500 rpm. Sucrose Acetate Isobutyrate (SAIB) was transferred into a glass container. Sieved cellulose acetate butyrate (CAB) was added to the bottle while mixing. After mixing for approximately 5 minutes, triacetin was added and mixed until the mass became clear. Butylated hydroxytoluene (BHT) was dissolved first in isopropyl myristate (IPM) and added into bottle with mixing. Hydroxyethyl cellulose (HEC) was added into the bottle and mixed well. In addition, formulations containing Labrafil M2125CS and/or sodium dodecyl sulfate (SDS) were added here and mixed well. Finally colliodal silicon dioxide (Cab-o-Sil®) was added into the bottle and were mixed to complete the formulation. Hydromorphone HCl was added into placebo formulation and dispersed well. Active formulations were then filled into size 0 gelatin capsules.

For all formulations BHT was included at a concentration of 0.02% w/w relative to the total weight of the placebo, i.e., the total weight of all components except hydromorphone HCl. The concentration of BHT is not taken into account in the % w/w calculations provided in Table 16 below.

TABLE 16

| Formulation | SAIB/Triacetin | | | | | | | Hydromorphone | | |
|---|---|---|---|---|---|---|---|---|---|---|
| ID | (ratio) | SAIB | Triacetin | IPM | CAB | SDS | Labrafil | HCl | HEC | SiO2 |
| 13 | 1.8 | 41.78 | 23.21 | 16.95 | 3.77 | 0.94 | 2.83 | 5.82 | 1.88 | 2.83 |
| 14* | 1.8 | 41.78 | 23.21 | 11.30 | 7.53 | 0 | 0 | 5.82 | 7.53 | 2.83 |
| 15 | 1.2 | 32.65 | 27.21 | 16.08 | 3.57 | 0 | 0.00 | 10.67 | 7.15 | 2.68 |
| 16* | 1.2 | 33.39 | 27.83 | 11.30 | 7.53 | 0.94 | 2.83 | 5.82 | 7.53 | 2.83 |
| 17 | 1.8 | 43.64 | 24.25 | 10.72 | 3.57 | 0 | 2.68 | 10.67 | 1.79 | 2.68 |
| 18* | 1.2 | 33.13 | 27.61 | 16.08 | 7.15 | 0.89 | 0 | 10.67 | 1.79 | 2.68 |
| 19* | 1.6 | 41.15 | 25.72 | 11.30 | 5.65 | 0 | 0.00 | 5.82 | 7.53 | 2.83 |
| 20* | 1.2 | 34.42 | 28.68 | 11.30 | 5.65 | 0.94 | 2.83 | 5.82 | 7.53 | 2.83 |
| 21 | 1.2 | 34.11 | 28.42 | 16.08 | 5.36 | 0.89 | 0.00 | 10.67 | 1.79 | 2.68 |
| 22 | 1.5 | 38.43 | 25.62 | 14.13 | 4.71 | 0.94 | 2.83 | 5.82 | 5.65 | 1.88 |
| 23 | 1.5 | 38.43 | 25.62 | 14.13 | 4.71 | 0.94 | 2.83 | 5.82 | 5.65 | 1.88 |
| (GLP, 8 mg) | | | | | | | | | | |
| 24 | 1.5 | 38.43 | 25.62 | 14.13 | 4.71 | 0.94 | 2.83 | 5.82 | 5.65 | 1.88 |
| (GLP, 16 mg) | | | | | | | | | | |

*final formulation could not be prepared due to high viscosity

Dissolution Testing

Dissolution experiments were performed using 2-phase medium in a USP Apparatus 2. The capsules were placed in stainless steel (316SS) wire spiral capsule sinkers for dissolution testing. The dissolution parameters were as follows:

Dissolution medium: 750 ml 0.1N HCl for the first 2 hours, add 250 ml 0.2 M phosphate buffer to achieve a final pH of 6.8; Paddle speed: 100 rpm; Vessel temperature: 37 C. Sampling time points: 0.25, 0.5, 1, 2, 3, 6, 10, 12, 18 and 24 hours. Sampling volume: 1 mL.

The HPLC parameters were as follows: Mobile phase A: 0.5% sodium dodecyl sulfate 1% glacial acetic acid, 20% acetonitrile; Mobile phase B: 100% acetonitrile; Mobile phase: 65% Mobile phase A and 35% Mobile phase B; 240 nm wavelength. Capsule number=2-4 capsules per testing.

Abuse Deterrence

Capsules from each composition were tested for abuse deterrence characteristics. The release rate of hydromorphone HCl was determined using an isocratic HPLC method at defined time points. The capsules were subjected to 60 mL of acidified 80-proof ethanol with vigorous shaking Each capsule was placed in a wide mouth round jar containing 36 mL of 0.1 N HCl and 24 mL 200-proof ethanol. The sample jar was placed in a shaking incubator maintained at 25° C. with 240 rpm shaking speed over the course of the 3 hour extraction test. The sampling time points were 0.5, 1, 2 and 3 hours. A 1 mL sample was taken at each time point and assayed using reverse-phase HPLC at 240 nm wavelength. The mobile phase included 0.35% (w/v) SDS/0.7% (v/v) acetic acid/44% (v/v) acetonitrile in water.

### Results

Dissolution Testing Results

The results of the dissolution experiments are provided in Tables 17 and 18 below.

TABLE 17

| | | Cumulative Drug Released (%) | | | | | | | | | |
|---|---|---|---|---|---|---|---|---|---|---|---|
| Dissolution | HMH (mg) | 0.25 hr | 0.5 hr | 1 hr | 2 hr | 3 hr | 6 hr | 10 hr | 12 hr | 18 hr | 24 hr |

Formulation 13

| | | | | | | | | | | | |
|---|---|---|---|---|---|---|---|---|---|---|---|
| #1 | 29.00 | 0.44 | 0.66 | 0.88 | 1.35 | 1.62 | 2.59 | 3.45 | 4.10 | 5.25 | 6.62 |
| #2 | 28.80 | 0.44 | 0.58 | 0.85 | 1.32 | 1.64 | 2.53 | 3.64 | 4.28 | 5.57 | 7.78 |
| #3 | 28.43 | 0.77 | 0.95 | 1.33 | 1.85 | 2.12 | 3.04 | 4.21 | 4.91 | 6.30 | 7.56 |
| #4 | 28.95 | 0.89 | 1.05 | 1.45 | 2.00 | 2.40 | 3.40 | 4.83 | 5.41 | 7.38 | 9.18 |
| Average | 28.80 | 0.6 | 0.8 | 1.1 | 1.6 | 1.9 | 2.9 | 4.0 | 4.7 | 6.1 | 7.8 |
| Std Dev | 0.26 | 0.2 | 0.2 | 0.3 | 0.3 | 0.4 | 0.4 | 0.6 | 0.6 | 0.9 | 1.1 |
| % RSD | 0.89 | 36.2 | 28.0 | 26.9 | 21.5 | 19.6 | 14.1 | 15.4 | 12.9 | 15.5 | 13.6 |

Formulation 15

| | | | | | | | | | | | |
|---|---|---|---|---|---|---|---|---|---|---|---|
| #1 | 28.49 | 1.27 | 1.85 | 2.95 | 5.38 | 7.40 | 16.07 | 29.88 | 36.12 | 51.86 | 64.22 |
| #2 | 28.02 | 1.20 | 1.80 | 3.06 | 5.83 | 8.28 | 17.56 | 31.03 | 37.41 | 53.85 | 67.02 |
| #3 | 27.17 | 1.19 | 1.66 | 2.83 | 5.55 | 8.10 | 18.67 | 35.00 | 42.26 | 59.55 | 72.15 |
| #4 | 27.74 | 1.00 | 1.62 | 2.75 | 5.35 | 7.62 | 16.19 | 29.23 | 35.11 | 50.34 | 62.61 |
| Average | 27.85 | 1.2 | 1.7 | 2.9 | 5.5 | 7.9 | 17.1 | 31.3 | 37.7 | 53.9 | 66.5 |
| Std Dev | 0.55 | 0.1 | 0.1 | 0.1 | 0.2 | 0.4 | 1.2 | 2.6 | 3.2 | 4.0 | 4.2 |
| % RSD | 1.99 | 9.7 | 6.1 | 4.6 | 4.0 | 5.2 | 7.2 | 8.3 | 8.4 | 7.5 | 6.3 |

Formulation 17

| | | | | | | | | | | | |
|---|---|---|---|---|---|---|---|---|---|---|---|
| #1 | 28.99 | 0.47 | 0.63 | 0.82 | 1.21 | 1.81 | 3.02 | 4.96 | 5.94 | 9.47 | 13.25 |
| #2 | 28.61 | 0.89 | 1.15 | 1.52 | 2.23 | 2.82 | 5.06 | 8.36 | 10.33 | 16.28 | 22.85 |
| #3 | 28.80 | 0.55 | 0.67 | 0.88 | 1.33 | 1.78 | 3.19 | 5.42 | 6.81 | 10.95 | 14.97 |
| #4 | 28.70 | 0.89 | 1.01 | 1.40 | 2.14 | 2.81 | 4.64 | 7.21 | 8.77 | 13.54 | 18.32 |
| Average | 28.78 | 0.7 | 0.9 | 1.2 | 1.7 | 2.3 | 4.0 | 6.5 | 8.0 | 12.6 | 17.3 |
| Std Dev | 0.16 | 0.2 | 0.3 | 0.4 | 0.5 | 0.6 | 1.0 | 1.6 | 2.0 | 3.0 | 4.2 |
| % RSD | 0.56 | 31.8 | 29.7 | 30.7 | 30.6 | 25.6 | 25.7 | 24.4 | 24.8 | 23.9 | 24.4 |

TABLE 18

| | | Cumulative Drug Released (%) | | | | | | | | | |
|---|---|---|---|---|---|---|---|---|---|---|---|
| Dissolution | Hydromorphone (mg) | 0.25 hr | 0.5 hr | 1 hr | 2 hr | 3 hr | 6 hr | 10 hr | 12 hr | 18 hr | 24 hr |

Formulation 21

| | | | | | | | | | | | |
|---|---|---|---|---|---|---|---|---|---|---|---|
| #1 | 26.92 | 2.43 | 3.28 | 4.98 | 8.26 | 11.01 | 19.59 | 31.00 | 36.15 | 49.11 | 59.43 |
| #2 | 26.92 | 1.69 | 2.73 | 4.35 | 7.70 | 10.51 | 19.50 | 30.94 | 36.08 | 49.25 | 59.65 |
| #3 | 27.86 | 1.46 | 2.24 | 3.66 | 6.86 | 9.58 | 18.59 | 30.55 | 36.06 | 49.52 | 59.63 |
| #4 | 28.33 | 1.71 | 2.48 | 3.97 | 7.29 | 10.17 | 19.51 | 32.01 | 37.51 | 52.10 | 63.88 |
| Average | 27.51 | 1.8 | 2.7 | 4.2 | 7.5 | 10.3 | 19.3 | 31.1 | 36.4 | 50.0 | 60.6 |
| Std Dev | 0.71 | 0.4 | 0.4 | 0.6 | 0.6 | 0.6 | 0.5 | 0.6 | 0.7 | 1.4 | 2.2 |
| % RSD | 2.58 | 23.2 | 16.6 | 13.4 | 7.9 | 5.8 | 2.4 | 2.0 | 1.9 | 2.8 | 3.6 |

Formulation 22

| | | | | | | | | | | | |
|---|---|---|---|---|---|---|---|---|---|---|---|
| #1 | 31.65 | 1.40 | 2.22 | 4.05 | 9.16 | 13.75 | 28.02 | 42.13 | 47.63 | 60.62 | 70.72 |
| #2 | 31.03 | 1.33 | 2.21 | 4.16 | 9.39 | 14.19 | 28.13 | 42.16 | 47.40 | 60.29 | 69.70 |
| #3 | 31.34 | 2.49 | 3.92 | 7.27 | 14.96 | 21.07 | 36.15 | 49.44 | 54.54 | 65.25 | 73.11 |
| #4 | 31.08 | 1.10 | 1.81 | 3.33 | 7.53 | 11.67 | 25.47 | 39.74 | 45.42 | 58.37 | 68.35 |
| Average | 31.28 | 1.6 | 2.5 | 4.7 | 10.3 | 15.2 | 29.4 | 43.4 | 48.7 | 61.1 | 70.5 |
| Std Dev | 0.29 | 0.6 | 0.9 | 1.8 | 3.2 | 4.1 | 4.6 | 4.2 | 4.0 | 2.9 | 2.0 |
| % RSD | 0.91 | 39.3 | 37.0 | 37.2 | 31.6 | 26.9 | 15.8 | 9.7 | 8.2 | 4.8 | 2.9 |

Abuse Deterrence Results

The results of the abuse deterrence experiments are provided in Table 19 below.

TABLE 19

| Formulation ID# | % Accumulative Amount of Drug Released |
|---|---|
| 13 | 7.8 |
| 15 | 26.6 |
| 17 | 3.9 |
| 21 | 28.6 |
| 22 | 23.4 |
| 23 (GLP, 8 mg) | 51.6 |
| 23 | |
| 24 (GLP, 16 mg) | 44.8 |

### Example 8

### Preparation and Analysis of Additional Extended Release Hydromorphone HCl Compositions (Formulations 25-30)

Additional hydromorphone compositions were prepared and characterized with respect to dissolution profile and abuse deterrence characteristics as indicated below.

### Materials and Methods

The compositions were prepared to provide the compositions indicated in Table 20 (below). Composition component amounts are % w/w relative to the total weight of the formulation including hydromorphone HCl prior to encapsulation unless otherwise noted. The SAIB/triacetin ratio is noted.

The formulations were prepared in 100 g scale. SAIB was equilibrated in 60° C.±5° C. water bath, and triacetin was added and mixed for 30 minutes at 800 rpm. Then, CAB was slowly added to ensure immediate powder dispersion upon contact with the solvent, and allowed to mix at 1500 rpm until completely dissolved. A 0.25% w/v BHT in IPM stock solution was prepared. The mixture of BHT/IPM and remaining IPM was added and mixed at 1500 rpm for 30 minutes. The order of addition for the remaining components was as follows: SDS (where applicable), HEC, and Cab-o-Sil®. each excipient was mixed at 1500 rpm for 30 minutes. After the addition of Cab-o-Sil®, the mixture was homogenized at 6000 RPM for 10 minutes. Hydromorphone HCl (HMH) was added into the placebo formulation and dispersed well. Active formulations were then filled into size 0 gelatin capsules.

For all formulations BHT was included at a concentration of 0.02% w/w relative to the total weight of the placebo, i.e., the total weight of all components except hydromorphone HCl. The concentration of BHT is not taken into account in the % w/w calculations provided in Table 20 below.

TABLE 20

| Formulation ID | SAIB/Triacetin | HMH | SAIB | Triacetin | IPM | CAB | HEC | Cab-o-Sil ® | SDS | Labrafil |
|---|---|---|---|---|---|---|---|---|---|---|
| 25 | 1.5 | 5.82 | 40.69 | 27.12 | 14.13 | 4.71 | 5.65 | 1.88 | 0 | 0 |
| 26 | 1.5 | 5.82 | 40.66 | 27.11 | 14.13 | 4.71 | 5.65 | 1.88 | 0.047 | 0 |
| 27 | 1.5 | 5.82 | 40.26 | 26.84 | 14.13 | 4.71 | 5.65 | 1.88 | 0 | 0.71 |
| 28 | 1.5 | 5.82 | 39.84 | 26.56 | 14.13 | 4.71 | 5.65 | 1.88 | 0 | 1.41 |
| 29 | 1.5 | 5.82 | 39.56 | 26.37 | 14.13 | 4.71 | 5.65 | 1.88 | 0 | 1.88 |
| 30 | 1.5 | 5.82 | 39.27 | 26.18 | 14.13 | 4.71 | 5.65 | 1.88 | 0 | 2.35 |

Dissolution Testing

Dissolution experiments were performed using 2-phase medium in a USP Apparatus 2. The capsules were placed in stainless steel (316SS) wire spiral capsule sinkers for dissolution testing. The dissolution parameters were as follows:

Dissolution medium: 750 ml 0.1N HCl for the first 2 hours, add 250 ml 0.2 M phosphate buffer to achieve a final pH of 6.8; Paddle speed: 100 rpm; Vessel temperature: 37 C. Sampling time points: 0.25, 0.5, 1, 2, 3, 6, 10, 12, 18 and 24 hours. Sampling volume: 1 mL.

The HPLC parameters were as follows: Mobile phase A: 0.5% sodium dodecyl sulfate 1% glacial acetic acid, 20% acetonitrile; Mobile phase B: 100% acetonitrile; Mobile phase: 65% Mobile phase A and 35% Mobile phase B; 240 nm wavelength. Capsule number=2-4 capsules per testing.

Abuse Deterrence

Capsules from each composition were tested for abuse deterrence characteristics. The release rate of hydromorphone HCl was determined using an isocratic HPLC method at defined time points. The capsules were subjected to 60 mL of acidified 80-proof ethanol with vigorous shaking Each capsule was placed in a wide mouth round jar containing 36 mL of 0.1 N HCl and 24 mL 200-proof ethanol. The sample jar was placed in a shaking incubator maintained at 25° C. with 240 rpm shaking speed over the course of the 3 hour extraction test. The sampling time points were 0.5, 1, 2 and 3 hours. A 1 mL sample was taken at each time point and assayed using reverse-phase HPLC at 240 nm wavelength. The mobile phase included 0.35% (w/v) SDS/0.7% (v/v) acetic acid/44% (v/v) acetonitrile in water.

### Results

Dissolution Testing and Abuse Deterrence Results

The results of the dissolution and abuse deterrence experiments are provided in Tables 21 and 22 below. All formulations showed good abuse deterrence properties with less than 20% cumulative release after 3 hours ethanol extraction.

TABLE 21

| Formula- | Dissolution | | | | | | %, Cumulative |
|---|---|---|---|---|---|---|---|
| tion ID# | % | % | % | % | % | % | release after 3 |
| (32 mg) | 0.5 hr | 3 hr | 6 hr | 10 hr | 12 hr | 24 hr | hrs extraction |
| 25 | 2.6 | 7.2 | 11.6 | 15.8 | 18.6 | 32.7 | 15.9 |
| 26 | 3.6 | 9.9 | 15.6 | 23.3 | 26.4 | 44.8 | 15.2 |
| 27 | 1.5 | 3.7 | 5.8 | 8.7 | 10.6 | 20.9 | 11.3 |

TABLE 22

| Formula- | Dissolution | | | | | | %, Cumulative |
|---|---|---|---|---|---|---|---|
| tion ID# | % | % | % | % | % | % | release after 3 |
| (32 mg) | 0.5 hr | 3 hr | 6 hr | 10 hr | 12 hr | 24 hr | hrs extraction |
| 28 | 0.0 | 2.7 | 4.7 | 8.2 | 10.2 | 21.0 | 18.0 |
| 29 | 0.0 | 3.1 | 5.8 | 10.4 | 12.9 | 25.2 | 16.8 |
| 30 | 0.0 | 3.1 | 6.1 | 11.1 | 13.6 | 27.2 | 13.1 |

### Example 9

### Preparation and Analysis of Additional Extended Release Hydromorphone HCl Compositions (Formulations 31-34)

Additional hydromorphone compositions were prepared and characterized with respect to dissolution profile and abuse deterrence characteristics as indicated below.

### Materials and Methods

The compositions were prepared to provide the compositions indicated in Table 23 (below). Composition component amounts are % w/w relative to the total weight of the formulation including hydromorphone HCl prior to encapsulation unless otherwise noted. The SAIB/triacetin ratio is noted.

The placebo formulations were prepared in 150 g scale. Three stock solutions, SAIB/TA (1.50), SAIB/TA (1.35) and 0.6% w/v BHT in IPM, were prepared before the compounding procedure started. A bottle of Gelucire® 44/14 was heated at 70° C. and homogenized at 9600 rpm prior to the starting the preparation. The temperature of the process was maintained at 60° C.±5° C. SAIB/TA stock solution was added to a jar, and then pre-heated Gelucire® 44/14 solution was added. The mixture was put into a water bath, and mixed at 500 rpm. 0.6% BHT/IPM stock solution was transferred into a vial, the vial was then rinsed with the remaining IPM and added to the formulation. The solution was mixed to ensure uniformity. Cab-O-Sil®M-5P was then added and mixed at 500 rpm. After at least 30 minutes mixing, the mixture was homogenized for 5 minutes at 9600 rpm. Sieved CAB was then added to the mixture and mixed at an initial speed of 500 rpm followed by mixing at 1500 rpm for approximately 30 total minutes or until all CAB particles were completely dissolved. Then HEC was added last, and mixed at 1500 rpm. Part of the placebo formulation was transferred into a separate bottle and hydromorphone HCl was introduced into the mixture and dispersed well to make 100 g active formulations. Active formulations were then filled into size 0 gelatin capsules.

For all formulations BHT was included at a concentration of 0.02% w/w relative to the total weight of the placebo, i.e., the total weight of all components except hydromorphone HCl. The concentration of BHT is not taken into account in the % w/w calculations provided in Table 23 below.

TABLE 23

| Formula- | Dose (mg)/ | Formulation Composition | | | | | | | |
|---|---|---|---|---|---|---|---|---|---|
| tion | Fill Weight | | | Triacetin | | | | | Gelucire ® |
| ID# | (mg)/Conc. | SAIB/TA | SAIB | (TA) | IPM | CAB | HEC | Cab-o-sil ® | 44/14 |
| 31 | 32/550/5.82% | 1.5 | 40.716 | 27.144 | 14.1 | 4.7 | 5.64 | 1.88 | 0 |
| 32 | 32/550/5.82% | 1.5 | 40.6032 | 27.0688 | 14.1 | 4.7 | 5.64 | 1.88 | 0.188 |
| 33 | 32/550/5.82% | 1.5 | 40.434 | 26.956 | 14.1 | 4.7 | 5.64 | 1.88 | 0.47 |
| 34 | 32/550/5.82% | 1.5 | 40.2648 | 26.8432 | 14.1 | 4.7 | 5.64 | 1.88 | 0.752 |

Dissolution Testing

Dissolution experiments were performed using 2-phase medium in a USP Apparatus 2. The capsules were placed in stainless steel (316SS) wire spiral capsule sinkers for dissolution testing. The dissolution parameters were as follows:

Dissolution medium: 750 ml 0.1N HCl for the first 2 hours; 250 ml 0.2 M phosphate buffer was added to achieve a final pH of 6.8; Paddle speed: 100 rpm; Vessel temperature: 37 C. Sampling time points: 0.25, 0.5, 1, 2, 3, 6, 10, 12, 18 and 24 hours. Sampling volume: 1 mL.

The HPLC parameters were as follows: Mobile phase A: 0.5% sodium dodecyl sulfate 1% glacial acetic acid, 20% acetonitrile; Mobile phase B: 100% acetonitrile; Mobile phase: 65% Mobile phase A and 35% Mobile phase B; 240 nm wavelength. Capsule number=2-4 capsules per testing.

Abuse Deterrence

Capsules from each composition were tested for abuse deterrence characteristics. The release rate of hydromorphone HCl was determined using an isocratic HPLC method at defined time points. The capsules were subjected to 60 mL of acidified 80-proof ethanol with vigorous shaking Each capsule was placed in a wide mouth round jar containing 36 mL of 0.1 N HCl and 24 mL 200-proof ethanol. The sample jar was placed in a shaking incubator maintained at 25° C. with 240 rpm shaking speed over the course of the 3 hour extraction test. The sampling time points were 0.5, 1, 2 and 3 hours. A 1 mL sample was taken at each time point and assayed using reverse-phase HPLC at 240 nm wavelength. The mobile phase included 0.35% (w/v) SDS/0.7% (v/v) acetic acid/44% (v/v) acetonitrile in water.

### Results

Dissolution Testing and Abuse Deterrence Results

The results of the dissolution and abuse deterrence experiments are provided in Table 24 below. All formulations showed good abuse deterrence properties with less than 15% cumulative release after 3 hours ethanol extraction.

TABLE 24

| Formula- | Dissolution Results (% Cumulative Drug Released) | | | | | | | | | | %, Cumulative |
|---|---|---|---|---|---|---|---|---|---|---|---|
| tion | 0.25 | 1.5 | 1 | 2 | 3 | 6 | 10 | 12 | 18 | 24 | release after 3 |
| ID# | hr | hr | hr | hr | hr | hr | hr | hr | hr | hr | hrs extraction |
| 31 | 0 | 0 | 1 | 2 | 2 | 4 | 7 | 8 | 13 | 17 | 12 |
| 32 | 1 | 1 | 1 | 2 | 3 | 5 | 8 | 9 | 13 | 17 | 7 |
| 33 | 1 | 1 | 2 | 3 | 6 | 12 | 20 | 24 | 34 | 43 | 8 |
| 34 | 2 | 3 | 5 | 9 | 13 | 25 | 39 | 45 | 59 | 71 | 13 |

### Example 10

### Preparation and Analysis of Additional Extended Release Hydromorphone HCl Compositions (Formulations 35-66)

Additional hydromorphone compositions were prepared and characterized with respect to dissolution profile and abuse deterrence characteristics as indicated below.

Materials and Methods

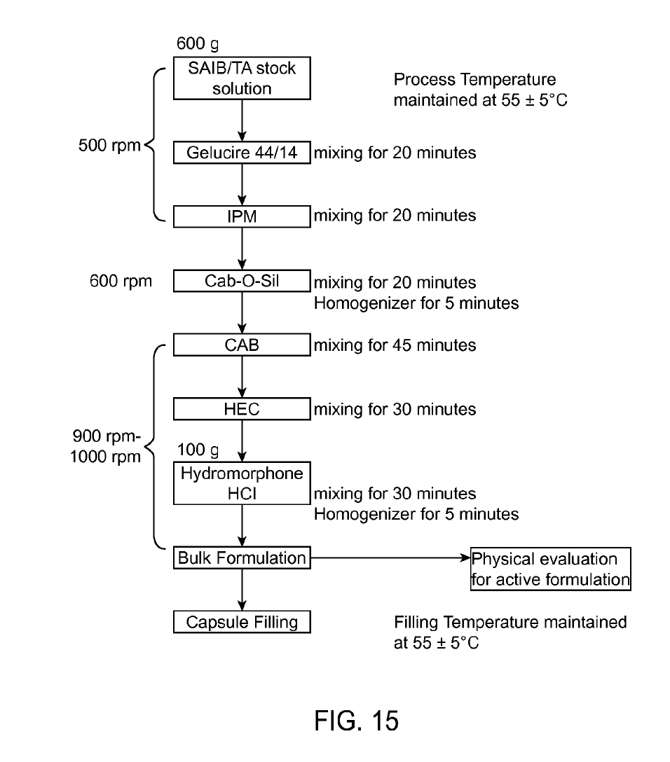

The compositions were prepared to provide the compositions indicated in Tables 25-27 (below). Component amounts are % w/w relative to the total weight of the formulation including hydromorphone HCl prior to encapsulation, unless otherwise indicated. The materials and methods applicable to the preparation of these formulations are provided in FIG. 15. Active formulations were filled into size 2 HPMC capsules.

TABLE 25

| | | | | | | | Cab- | |
|---|---|---|---|---|---|---|---|---|
| Formula- | Hydromorphone | | | | | | o- | Gelucire ® |
| tion ID | HCl | SAIB | Triacetin | CAB | IPM | HEC | Sil ® | 44/14 |
| 35 | 5.82% | 45.16% | 32.26% | 5.65% | 1.88% | 5.65% | 2.83% | 0.75% |
| 36 | 5.82% | 46.04% | 32.88% | 5.65% | 1.88% | 5.65% | 1.88% | 0.19% |
| 37 | 5.82% | 42.41% | 30.29% | 5.65% | 7.53% | 5.65% | 1.88% | 0.75% |
| 38 | 5.82% | 45.05% | 32.18% | 5.65% | 4.71% | 3.77% | 2.35% | 0.47% |
| 39 | 5.82% | 47.91% | 34.22% | 5.65% | 1.88% | 1.88% | 1.88% | 0.75% |
| 40 | 5.82% | 42.19% | 30.14% | 5.65% | 7.53% | 5.65% | 2.83% | 0.19% |
| 41 | 5.82% | 44.06% | 31.47% | 5.65% | 7.53% | 1.88% | 2.83% | 0.75% |
| 42 | 5.82% | 44.94% | 32.10% | 5.65% | 7.53% | 1.88% | 1.88% | 0.19% |
| 43 | 5.82% | 47.69% | 34.06% | 5.65% | 1.88% | 1.88% | 2.83% | 0.19% |

TABLE 26

| | | | | | | | Cab- | |
|---|---|---|---|---|---|---|---|---|
| Formula- | Hydromorphone | | | | | | o- | Gelucire ® |
| tion ID | HCl | SAIB | Triacetin | CAB | IPM | HEC | sil ® | 44/14 |
| 44 | 5.82% | 41.34% | 41.34% | 5.65% | 1.88% | 1.88% | 1.88% | 0.19% |
| 45 | 5.82% | 38.05% | 38.05% | 5.65% | 7.53% | 1.88% | 2.83% | 0.19% |
| 46 | 5.82% | 38.24% | 38.24% | 5.65% | 7.53% | 1.88% | 1.88% | 0.75% |
| 47 | 5.82% | 38.61% | 38.61% | 5.65% | 4.71% | 3.77% | 2.35% | 0.47% |
| 48 | 5.82% | 36.64% | 36.64% | 5.65% | 7.53% | 5.65% | 1.88% | 0.19% |
| 49 | 5.82% | 38.99% | 38.99% | 5.65% | 1.88% | 5.65% | 2.83% | 0.19% |
| 50 | 5.82% | 39.18% | 39.18% | 5.65% | 1.88% | 5.65% | 1.88% | 0.75% |
| 51 | 5.82% | 40.59% | 40.59% | 5.65% | 1.88% | 1.88% | 2.83% | 0.75% |
| 52 | 5.82% | 35.88% | 35.88% | 5.65% | 7.53% | 5.65% | 2.83% | 0.75% |

TABLE 27

| | | | | | | | Cab- | |
|---|---|---|---|---|---|---|---|---|
| Formula- | Hydromorphone | | | | | | o- | Gelucire ® |
| tion ID | HCl | SAIB | Triacetin | CAB | IPM | HEC | sil ® | 44/14 |
| 53 | 5.82% | 42.38% | 35.32% | 5.65% | 4.71% | 3.77% | 1.88% | 0.47% |
| 54 | 5.82% | 42.12% | 35.10% | 5.65% | 4.71% | 3.77% | 2.35% | 0.47% |
| 55 | 5.82% | 42.12% | 35.10% | 5.65% | 4.71% | 3.77% | 2.35% | 0.47% |
| 56 | 5.82% | 41.10% | 34.25% | 5.65% | 4.71% | 5.65% | 2.35% | 0.47% |
| 57 | 5.82% | 43.67% | 36.39% | 5.65% | 1.88% | 3.77% | 2.35% | 0.47% |
| 58 | 5.82% | 40.58% | 33.82% | 5.65% | 7.53% | 3.77% | 2.35% | 0.47% |
| 59 | 5.82% | 42.12% | 35.10% | 5.65% | 4.71% | 3.77% | 2.35% | 0.47% |
| 60 | 5.82% | 42.12% | 35.10% | 5.65% | 4.71% | 3.77% | 2.35% | 0.47% |
| 61 | 5.82% | 41.87% | 34.89% | 5.65% | 4.71% | 3.77% | 2.83% | 0.47% |
| 62 | 5.82% | 41.97% | 34.97% | 5.65% | 4.71% | 3.77% | 2.35% | 0.75% |
| 63 | 5.82% | 42.12% | 35.10% | 5.65% | 4.71% | 3.77% | 2.35% | 0.47% |
| 64 | 5.82% | 42.12% | 35.10% | 5.65% | 4.71% | 3.77% | 2.35% | 0.47% |
| 65 | 5.82% | 42.28% | 35.23% | 5.65% | 4.71% | 3.77% | 2.35% | 0.19% |
| 66 | 5.82% | 43.15% | 35.96% | 5.65% | 4.71% | 1.88% | 2.35% | 0.47% |

Dissolution Testing

Dissolution experiments were performed using 2-phase medium in a USP Apparatus 2. The capsules were placed in stainless steel (316SS) wire spiral capsule sinkers for dissolution testing. The dissolution parameters were as follows:

Dissolution medium: 750 ml 0.1N HCl for the first 2 hours; 250 ml 0.2 M phosphate buffer was added to achieve a final pH of 6.8; Paddle speed: 100 rpm; Vessel temperature: 37 C. Sampling time points: 0.25, 0.5, 1, 2, 3, 6, 10, 12, 18 and 24 hours. Sampling volume: 1 mL.

The HPLC parameters were as follows: Mobile phase A: 0.5% sodium dodecyl sulfate 1% glacial acetic acid, 20% acetonitrile; Mobile phase B: 100% acetonitrile; Mobile phase: 65% Mobile phase A and 35% Mobile phase B; 240 nm wavelength. Capsule number=2-4 capsules per testing.

Abuse Deterrence

Capsules from each composition were tested for abuse deterrence characteristics. The release rate of hydromorphone HCl was determined using an isocratic HPLC method at defined time points. The capsules were subjected to 60 mL of acidified 80-proof ethanol with vigorous shaking Each capsule was placed in a wide mouth round jar containing 36 mL of 0.1 N HCl and 24 mL 200-proof ethanol. The sample jar was placed in a shaking incubator maintained at 25° C. with 240 rpm shaking speed over the course of the 3 hour extraction test. The sampling time points were 0.5, 1, 2 and 3 hours. A 1 mL sample was taken at each time point and assayed using reverse-phase HPLC at 240 nm wavelength. The mobile phase included 0.35% (w/v) SDS/0.7% (v/v) acetic acid/44% (v/v) acetonitrile in water.

### Results

Dissolution Testing and Abuse Deterrence Results

The results of the dissolution and abuse deterrence experiments are provided in Table 28 below.

TABLE 28

| | | 2-phase | | | | | | | | | | |
|---|---|---|---|---|---|---|---|---|---|---|---|---|
| | | Dissolution | | | | | | | | | | %, |
| | | %, | %, | %, | %, | %, | %, | %, | %, | %, | %, | Abuse |
| Formula- | | 0.25 | 0.5 | 1 | 2 | 3 | 6 | 10 | 12 | 18 | 24 | at 3- |
| tion ID | t = 0 | hr | hr | hr | hrs | hrs | hrs | hrs | hrs | hrs | hrs | hour |
| 35 | Mean | 1 | 2 | 3 | 4 | 6 | 14 | 25 | 30 | 42 | 52 | 13 |
| | SD | 1 | 1 | 1 | 2 | 2 | 5 | 8 | 10 | 11 | 12 | |
| 36 | Mean | 0 | 1 | 1 | 2 | 2 | 2 | 3 | 3 | 4 | 5 | 15 |
| | SD | 0 | 1 | 0 | 1 | 0 | 1 | 1 | 1 | 1 | 1 | |
| 37 | Mean | 2 | 4 | 6 | 9 | 12 | 20 | 28 | 31 | 40 | 47 | 21 |
| | SD | 1 | 1 | 1 | 1 | 1 | 2 | 4 | 4 | 5 | 7 | |
| 38 | Mean | 0 | 1 | 1 | 1 | 2 | 2 | 3 | 3 | 4 | 5 | 16 |
| | SD | 0 | 0 | 0 | 0 | 1 | 0 | 0 | 0 | 1 | 1 | |
| 39 | Mean | 1 | 3 | 4 | 7 | 10 | 19 | 31 | 36 | 50 | 61 | 17 |
| | SD | 0 | 1 | 1 | 2 | 3 | 4 | 6 | 6 | 8 | 8 | |
| 40 | Mean | 2 | 2 | 3 | 4 | 6 | 8 | 9 | 9 | 11 | 13 | 10 |
| | SD | 0 | 1 | 0 | 1 | 2 | 1 | 1 | 2 | 2 | 3 | |
| 41 | Mean | 2 | 3 | 3 | 4 | 8 | 11 | 15 | 17 | 24 | 29 | 9 |
| | SD | 0 | 1 | 0 | 1 | 2 | 2 | 3 | 3 | 5 | 7 | |
| 42 | Mean | 2 | 2 | 2 | 3 | 4 | 5 | 5 | 6 | 6 | 6 | 5 |
| | SD | 0 | 0 | 0 | 0 | 1 | 1 | 1 | 1 | 1 | 1 | |
| 43 | Mean | 2 | 2 | 2 | 2 | 4 | 5 | 5 | 5 | 5 | 5 | 19 |
| | SD | 0 | 0 | 0 | 1 | 1 | 1 | 1 | 1 | 1 | 1 | |
| 44 | Mean | 0 | 1 | 1 | 1 | 1 | 2 | 2 | 2 | 2 | 3 | 14 |
| | SD | 0 | 0 | 0 | 0 | 0 | 1 | 0 | 0 | 1 | 1 | |
| 45 | Mean | 0 | 1 | 1 | 2 | 2 | 3 | 4 | 4 | 5 | 6 | 7 |
| | SD | 1 | 0 | 1 | 1 | 0 | 1 | 1 | 1 | 2 | 2 | |
| 46 | Mean | 1 | 3 | 4 | 7 | 9 | 18 | 28 | 32 | 44 | 54 | 14 |
| | SD | 1 | 1 | 1 | 1 | 2 | 3 | 4 | 5 | 6 | 7 | |
| 47 | Mean | 0 | 0 | 1 | 2 | 2 | 3 | 5 | 6 | 8 | 10 | 15 |
| | SD | 0 | 0 | 1 | 1 | 1 | 1 | 1 | 1 | 2 | 2 | |
| 48 | Mean | 0 | 1 | 1 | 2 | 3 | 4 | 5 | 6 | 7 | 9 | 11 |
| | SD | 0 | 0 | 0 | 1 | 1 | 1 | 1 | 1 | 2 | 1 | |
| 49 | Mean | 0 | 1 | 2 | 3 | 3 | 5 | 6 | 7 | 10 | 13 | 34 |
| | SD | 0 | 0 | 0 | 1 | 1 | 1 | 2 | 2 | 2 | 3 | |
| 50 | Mean | 1 | 5 | 10 | 18 | 25 | 45 | 62 | 68 | 83 | 93 | 30 |
| | SD | 1 | 1 | 2 | 3 | 3 | 4 | 3 | 3 | 2 | 2 | |
| 51 | Mean | 0 | 1 | 2 | 2 | 3 | 6 | 11 | 14 | 23 | 32 | 19 |
| | SD | 0 | 1 | 1 | 1 | 1 | 2 | 3 | 4 | 5 | 6 | |
| 52 | Mean | 0 | 2 | 5 | 8 | 11 | 21 | 31 | 35 | 46 | 54 | 13 |
| | SD | 0 | 1 | 1 | 2 | 3 | 4 | 6 | 6 | 7 | 7 | |
| 53 | Mean | 0 | 0 | 1 | 1 | 2 | 3 | 5 | 6 | 9 | 12 | 10 |
| | SD | 0 | 0 | 1 | 1 | 0 | 0 | 1 | 1 | 1 | 2 | |
| 54 | Mean | 0 | 0 | 1 | 2 | 2 | 4 | 5 | 6 | 8 | 10 | 12 |
| | SD | 0 | 1 | 1 | 1 | 1 | 1 | 1 | 2 | 2 | 2 | |
| 55 | Mean | 1 | 1 | 2 | 2 | 3 | 4 | 5 | 6 | 9 | 12 | 10 |
| | SD | 1 | 0 | 1 | 1 | 0 | 1 | 1 | 1 | 1 | 2 | |
| 56 | Mean | 0 | 0 | 2 | 2 | 3 | 5 | 7 | 8 | 12 | 16 | 14 |
| | SD | 0 | 0 | 1 | 1 | 0 | 1 | 1 | 2 | 1 | 2 | |
| 57 | Mean | 0 | 0 | 1 | 2 | 2 | 3 | 4 | 5 | 6 | 8 | 18 |
| | SD | 0 | 0 | 1 | 1 | 1 | 1 | 1 | 2 | 2 | 2 | |
| 58 | Mean | 0 | 1 | 1 | 2 | 2 | 4 | 6 | 7 | 10 | 12 | 9 |
| | SD | 0 | 1 | 0 | 1 | 1 | 1 | 2 | 2 | 2 | 3 | |
| 59 | Mean | 0 | 1 | 1 | 2 | 2 | 3 | 5 | 5 | 7 | 9 | 13 |
| | SD | 0 | 1 | 1 | 1 | 1 | 1 | 1 | 2 | 2 | 2 | |
| 60 | Mean | 1 | 1 | 1 | 2 | 2 | 3 | 5 | 5 | 8 | 9 | 12 |
| | SD | 1 | 0 | 1 | 1 | 1 | 1 | 2 | 2 | 2 | 2 | |
| 61 | Mean | 1 | 1 | 2 | 3 | 4 | 5 | 7 | 7 | 10 | 12 | 13 |
| | SD | 1 | 1 | 1 | 1 | 1 | 2 | 2 | 2 | 3 | 3 | |
| 62 | Mean | 1 | 2 | 3 | 4 | 6 | 13 | 23 | 27 | 38 | 46 | 15 |
| | SD | 0 | 1 | 1 | 1 | 2 | 4 | 6 | 7 | 7 | 8 | |
| 63 | Mean | 0 | 0 | 1 | 2 | 2 | 4 | 5 | 5 | 8 | 10 | 11 |
| | SD | 0 | 0 | 1 | 0 | 1 | 1 | 1 | 1 | 1 | 1 | |
| 64 | Mean | 0 | 0 | 1 | 1 | 2 | 2 | 4 | 4 | 6 | 7 | 15 |
| | SD | 0 | 0 | 1 | 0 | 0 | 1 | 1 | 1 | 1 | 1 | |
| 65 | Mean | 0 | 1 | 1 | 1 | 2 | 3 | 4 | 4 | 6 | 7 | 12 |
| | SD | 0 | 1 | 0 | 1 | 1 | 1 | 1 | 1 | 2 | 2 | |
| 66 | Mean | 0 | 0 | 1 | 1 | 1 | 2 | 3 | 3 | 4 | 5 | 13 |
| | SD | 0 | 1 | 0 | 0 | 1 | 1 | 1 | 1 | 1 | 1 | |

No apparent phase separation was observed for the above formulations. With respect to dissolution characteristics, each of formulations 35, 37, 39, 41, 46, 51, 52 and 62 exhibited dissolution profiles similar to the initial release range of the targeted profile.

With respect to abuse deterrence, all formulations showed good resistance to 40% ethanol extraction with the exception of 49 and 50 which exhibited drug extraction of ≧30% after 3 hours.

### Example 11

### Preparation and Analysis of Extended Release Hydrocodone Bitartrate Compositions (Formulations 67-78)

Hydrocodone bitartrate compositions were prepared and characterized with respect to dissolution profile and abuse deterrence characteristics as indicated below.

### Materials and Methods

The compositions were prepared to provide the compositions indicated in Tables 29 and 30 (below). For Tables 29 and 30, component amounts are % w/w relative to the total weight of the formulation including hydrocodone bitartrate prior to encapsulation, unless otherwise indicated. The SAIB/triacetin ratio is noted.

The placebo formulations were prepared in 300 g scale. Formulations were prepared as follows: several stock solutions, SAIB/TA at different ratio and 0.6% w/v BHT in IPM, were prepared before the compounding procedure started. The preparation took place in a 60° C.±5° C. water bath, and the temperature was maintained at 60° C.±5° C. during the preparation. SAIB/TA stock solution was transferred into a jar, and 0.6% BHT in IPM solution and the remaining IPM were added to the jar while mixing at 500 rpm. This combination was then mixed uniformly. Cab-o-Sil® M-5P was added and the combination was mixed for at least 2 hours. The mixture was homogenized at 9600 rpm for 5 minutes. Sieved CAB was then added into the jar and dissolved in the content of the jar at the elevated speed. HEC was then added into the jar and dispersed. Part of the placebo was transferred into a separate jar and hydrocodone bitartrate was introduced into the mixture and dispersed well to make 100 g active formulations. The active formulations were filled into size 0 gelatin capsules.

TABLE 29

| | | Formulation (containing 0.02% BHT) | | | | | | | | |
|---|---|---|---|---|---|---|---|---|---|---|
| | Dose (mg)/ | | | | | | | | Gelucire | |
| Formula- | Fill Weight | SAIB/ | | Triacetin | | | Cab-o- | | ® | Gelucire ® |
| tion ID# | (mg)/Conc. | TA | SAIB | (TA) | IPM | CAB | sil ® | HEC | 50/13 | 44/14 |
| 67 | 75/550/13.64% | 1.5 | 37.05 | 24.70 | 11.23 | 4.75 | 3.45 | 5.18 | 0 | 0 |
| 68 | 75/550/13.64% | 1.5 | 35.49 | 23.66 | 13.82 | 4.75 | 3.45 | 5.18 | 0 | 0 |
| 69 | 75/550/13.64% | 1.3 | 36.61 | 28.16 | 11.23 | 5.18 | 2.59 | 0 | 2.59 | 0 |
| 70 | 75/550/13.64% | 1.3 | 35.14 | 27.03 | 11.23 | 5.18 | 2.59 | 5.18 | 0 | 0 |
| 71 | 75/550/13.64% | 1.3 | 33.68 | 25.91 | 13.82 | 5.18 | 2.59 | 5.18 | 0 | 0 |
| 72 | 75/550/13.64% | 1.5 | 38.86 | 25.91 | 11.23 | 5.18 | 2.59 | 0 | 2.59 | 0 |
| 73 | 75/550/13.64% | 1.4 | 36.77 | 26.27 | 12.52 | 4.32 | 2.59 | 2.59 | 1.30 | 0 |
| 74 | 75/550/13.64% | 1.5 | 39.51 | 26.34 | 12.95 | 5.18 | 2.16 | 0 | 0 | 0.22 |
| 75 | 75/550/13.64% | 1.5 | 39.25 | 26.17 | 12.95 | 5.18 | 2.16 | 0 | 0 | 0.65 |

TABLE 30

| Formula- | | | | Triacetin | | | | | Cab-o- | Gelucire ® | |
|---|---|---|---|---|---|---|---|---|---|---|---|
| tion ID | SAIB/TA | API | SAIB | (TA) | CAB | BHT | IPM | HEC | sil ® | 44/14 | Fill wt. |
| 76 | 1.5 | 13.64% | 38.02% | 25.35% | 4.75% | 0.02% | 12.95% | 2.59% | 2.16% | 0.52% | 100.00% |
| 77 | 1.5 | 13.64% | 37.87% | 25.25% | 4.75% | 0.02% | 12.95% | 2.59% | 2.16% | 0.78% | 100.00% |
| 78 | 1.5 | 13.64% | 37.71% | 25.14% | 4.75% | 0.02% | 12.95% | 2.59% | 2.16% | 1.04% | 100.00% |

API = hydrocodone bitartrate

Dissolution Testing

Dissolution experiments were performed using 2-phase medium in a USP Apparatus 2. The capsules were placed in stainless steel (316SS) wire spiral capsule sinkers for dissolution testing. The dissolution parameters were as follows: Dissolution medium: 750 ml 0.1N HCl for the first 2 hours; 250 ml 0.2 M phosphate buffer was added to achieve a final pH of 6.8; Paddle speed: 100 rpm; Vessel temperature: 37 C. Sampling time points: 0.5, 2, 3, 6, 12, 18 and 24 hours. Sampling volume: 1 mL.

The HPLC parameters were as follows: Mobile phase A: 0.5% sodium dodecyl sulfate 1% glacial acetic acid, 20% acetonitrile; Mobile phase B: 100% acetonitrile; Mobile phase: 65% Mobile phase A and 35% Mobile phase B; 240 nm wavelength. Capsule number=2-4 capsules per testing.

Abuse Deterrence

Capsules from each composition were tested for abuse deterrence characteristics. The release rate of hydrocodone was determined using an isocratic HPLC method at defined time points. The capsules were subjected to 60 mL of acidified 80-proof ethanol with vigorous shaking Each capsule was placed in a wide mouth round jar containing 36 mL of 0.1 N HCl and 24 mL 200-proof ethanol. The sample jar was placed in a shaking incubator maintained at 25° C. with 240 rpm shaking speed over the course of the 3 hour extraction test. The sampling time points were 0.5, 1 and 3 hours. A 1 mL sample was taken at each time point and assayed using reverse-phase HPLC at 240 nm wavelength. The mobile phase included 0.35% (w/v) SDS/0.7% (v/v) acetic acid/47% (v/v) acetonitrile in water.

### Results

Dissolution Testing and Abuse Deterrence Results

The results of the dissolution and abuse deterrence experiments are provided in Tables 31 and 32 below.

TABLE 31

| | | | | | | | |
|---|---|---|---|---|---|---|---|
| Time (hrs) | 0.5 | 2 | 3 | 6 | 12 | 18 | 24 |

Formulation 76

| | | | | | | | |
|---|---|---|---|---|---|---|---|
| #1 | 1 | 2 | 3 | 4 | 8 | 12 | 17 |
| #2 | 1 | 2 | 3 | 4 | 8 | 12 | 16 |
| #3 | 1 | 2 | 3 | 4 | 8 | 12 | 17 |
| #4 | 1 | 2 | 3 | 4 | 8 | 14 | 18 |
| Average | 1 | 2 | 3 | 4 | 8 | 13 | 17 |
| Std Dev | 0 | 0 | 0 | 0 | 0 | 1 | 1 |
| % RSD | 0 | 0 | 0 | 0 | 0 | 8 | 5 |

Formulation 77

| | | | | | | | |
|---|---|---|---|---|---|---|---|
| #1 | 4 | 8 | 10 | 14 | 24 | 32 | 40 |
| #2 | 4 | 9 | 11 | 15 | 26 | 34 | 42 |
| #3 | 4 | 9 | 11 | 15 | 24 | 32 | 38 |
| #4 | 4 | 9 | 10 | 15 | 24 | 32 | 38 |
| Average | 4 | 9 | 11 | 15 | 25 | 33 | 40 |
| Std Dev | 0 | 1 | 1 | 1 | 1 | 1 | 2 |
| % RSD | 0 | 6 | 5 | 3 | 4 | 3 | 5 |

Formulation 78

| | | | | | | | |
|---|---|---|---|---|---|---|---|
| #1 | 11 | 27 | 33 | 44 | 58 | 67 | 74 |
| #2 | 10 | 25 | 30 | 41 | 54 | 64 | 72 |
| #3 | 10 | 27 | 33 | 44 | 59 | 69 | 76 |
| #4 | 13 | 32 | 39 | 50 | 65 | 75 | 82 |
| Average | 11 | 28 | 34 | 45 | 59 | 69 | 76 |
| Std Dev | 1 | 3 | 4 | 4 | 5 | 5 | 4 |
| % RSD | 13 | 11 | 11 | 8 | 8 | 7 | 6 |

TABLE 32

| | Dissolution Results (% Cumulative Drug Released) | | | | | | | %, Cumulative |
|---|---|---|---|---|---|---|---|---|
| | 0.5 | 2 | 3 | 6 | 12 | 18 | 24 | release after 3 |
| Formulation ID# | hr | hr | hr | hr | hr | hr | hr | hrs extraction |
| 67 | 1 | 3 | 3 | 4 | 7.8 | 11 | 14 | 15 |
| 68 | 1 | 2 | 3 | 5 | 9.8 | 15 | 21 | 9 |
| 69 | 27 | 54 | 62 | 78 | 94 | 97 | 98 | 38 |
| 70 | 0 | 3 | 3 | 6 | 11 | 17 | 20 | 12 |
| 71 | 0 | 3 | 4 | 7 | 15 | 23 | 31 | 11 |
| 72 | 22 | 46 | 55 | 71 | 90 | 96 | 98 | 34 |
| 73 | 18 | 42 | 49 | 63 | 81 | 89 | 93 | 37 |
| 74 | 0 | 1 | 1 | 2 | 4 | 5 | 6 | 2 |
| 75 | 1 | 3 | 4 | 5 | 9 | 12 | 15 | 4 |
| 76 | 1 | 2 | 3 | 4 | 8 | 13 | 17 | 7 |
| 77 | 4 | 9 | 11 | 15 | 25 | 33 | 40 | 12 |
| 78 | 11 | 28 | 34 | 45 | 59 | 69 | 76 | 15 |

For formulations 76 through 78, the results indicate a linear relationship between the amount of Gelucire® 44/14 in the formulation and the cumulative amount of hydrocodone bitartrate released from the formulation at a particular time point. These results also indicate an increase in the % cumulative release under the tested abuse conditions correlated with increasing amounts of Gelucire® 44/14. In contrast, an increase in the amount of Cab-o-Sil® in the formulations correlated with a decrease in the % cumulative release under the tested abuse conditions.

### Example 12

### Preparation and Analysis of Extended Release Amphetamine Compositions (Formulations 79-81)

Amphetamine compositions were prepared and characterized with respect to dissolution profile and abuse deterrence characteristics as indicated below.

### Materials and Methods

The compositions were prepared to provide the compositions indicated in Table 33 below. Component amounts are % w/w relative to the total weight of the formulation including amphetamine sulfate prior to encapsulation, unless otherwise indicated.

The placebo formulations were prepared in 150 g scale. Formulations were prepared as follows: stock solutions, SAIB/TA at different ratio and 0.6% w/v BHT in IPM, were prepared before the compounding procedure started. The preparation took place in a 60° C.±5° C. water bath, and the temperature was maintained at 60° C.±5° C. during the preparation. SAIB/TA stock solution was transferred into a jar. Sieved CAB was added into the jar and dispersed and dissolved in the solution at an elevated speed. 0.6% BHT in IPM and IPM was added to the jar and mixed uniformly. Gelucire® 50/13 was added to the content in the jar and mixed uniformly. Cab-o-Sil® was added and mixed to disperse uniformly. Part of the placebo was transferred into a separate jar and amphetamine sulfate was introduced into the mixture and dispersed well to make 100 g active formulations. The active formulations were filled into size 0 gelatin capsules.

TABLE 33

| | | D- | Formulation (containing 0.02% BHT) | | | | | | | |
|---|---|---|---|---|---|---|---|---|---|---|
| Formulation | | Amp | SAIB/ | | | | | | Cab-o-sil ® | Gelucire ® |
| ID# | API | Conc. % | Triacetin | SAIB % | TA % | IPM % | CAB % | HEC % | % | 50/13, % |
| 79 | D-Amphetamine | 5.45 | 1.35 | 38.97 | 28.87 | 9.46 | 4.96 | 5.67 | 1.89 | 4.73 |
| | Sulfate | | | | | | | | | |
| 80 | D-Amphetamine | 5.45 | 1.35 | 38.16 | 28.26 | 16.07 | 4.96 | 0.00 | 2.36 | 4.73 |
| | Sulfate | | | | | | | | | |
| 81 | D-Amphetamine | 5.45 | 1.35 | 37.89 | 28.06 | 16.07 | 4.96 | 0.00 | 2.84 | 4.73 |
| | Sulfate | | | | | | | | | |

TA = Triacetin

Dissolution Testing

2-phase dissolution medium was utilized in a USP Apparatus 2. Capsules were placed in stainless steel (316SS) wire spiral capsule sinkers for dissolution testing. The dissolution parameters were as follows: Dissolution medium: 750 ml 0.1N HCl for the first 2 hours, followed by the addition of 200 ml 0.19M phosphate buffer to achieve a final pH of 6.0; Paddle speed: 50 rpm; Vessel temperature: 37° C. Sampling time points: 0.25, 0.5, 1, 1.5, 2, 3, 6, 9, 12 and 24 hours. Sampling volume: 1 mL. HPLC parameters were as follows: Mobile phase A: 5 mM 1-Decanesulfonic acid, sodium salt, 5 mM sodium phosphate monobasic, pH 2.5; Mobile phase B: 100% acetonitrile; Mobile phase: 67% Mobile phase A and 33% Mobile phase B; 210 nm wavelength. Capsule number=4 capsules per test.

Abuse Deterrence

Capsules from each composition were tested for abuse deterrence characteristics. The release rate of dextroamphetamine was determined using an isocratic HPLC method at defined time points. The capsules were subjected to 60 mL of acidified 80-proof ethanol with vigorous shaking Each capsule was placed in a wide mouth round jar containing 36 mL of 0.1 N HCl and 24 mL 200-proof of ethanol. The sample jar was placed in a shaking incubator maintained at 25° C. with 240 rpm shaking speed over the course of the 3 hour extraction test. The sampling time points were 0.5 and 3 hours. A 1 mL sample was taken at each time point and assayed using reverse-phase HPLC at 210 nm wavelength. The mobile phase included and 33% (v/v) acetonitrile in 67% (v/v) 5 mM 1-Decanesulfonic Acid, Na salt, 5 mM sodium phosphate, pH 2.5.

### Results

Dissolution Testing and Abuse Deterrence Results

The results of the dissolution and abuse deterrence experiments are provided in Tables 34-36 and 37 respectively below.

TABLE 34
Formulation 79

| | D-Amphetamine | % Cumulative drug released (%) | | | | | | | | |
|---|---|---|---|---|---|---|---|---|---|---|
| Dissolution | Sulfate (mg) | 1 hr | 2 hr | 3 hr | 6 hr | 8 hr | 10 hr | 12 hr | 18 hr | 24 hr |
| #1 | 15.18 | 44.45 | 63.27 | 73.77 | 90.63 | 96.43 | 99.14 | 100.27 | 100.65 | 100.71 |
| #2 | 14.91 | 42.75 | 59.03 | 67.82 | 84.87 | 91.81 | 95.78 | 97.92 | 98.54 | 98.79 |
| #3 | 15.89 | 46.80 | 64.48 | 73.97 | 91.33 | 97.95 | 101.67 | 103.18 | 104.06 | 103.87 |
| #4 | 15.34 | 44.40 | 60.43 | 70.29 | 87.97 | 94.09 | 98.12 | 99.82 | 100.64 | 101.14 |
| Average | 15.3 | 44.6 | 61.8 | 71.5 | 88.7 | 95.1 | 98.7 | 100.3 | 101.0 | 101.1 |
| Std Dev | 0.4 | 1.7 | 2.5 | 3.0 | 2.9 | 2.7 | 2.4 | 2.2 | 2.3 | 2.1 |
| % RSD | 2.7 | 3.7 | 4.1 | 4.1 | 3.3 | 2.8 | 2.5 | 2.2 | 2.3 | 2.1 |

TABLE 35
Formulation 80

| | D-Amphetamine | % Cumulative drug released (%) | | | | | | | | | |
|---|---|---|---|---|---|---|---|---|---|---|---|
| Dissolution | Sulfate (mg) | 0.25 hr | 0.5 hr | 1 hr | 1.5 hr | 2 hr | 3 hr | 6 hr | 9 hr | 12 hr | 24 hr |
| #1 | 14.62 | 20.00 | 33.13 | 50.74 | 63.09 | 71.94 | 82.41 | 94.37 | 96.06 | 96.63 | 97.99 |
| #2 | 15.00 | 19.40 | 34.33 | 50.89 | 61.84 | 70.15 | 80.64 | 94.93 | 97.44 | 97.88 | 99.05 |
| #3 | 15.44 | 21.60 | 38.23 | 55.18 | 66.19 | 74.39 | 84.95 | 98.67 | 100.81 | 101.37 | 102.42 |
| #4 | 14.84 | 23.45 | 38.28 | 54.24 | 64.65 | 72.35 | 82.30 | 95.33 | 97.65 | 98.03 | 99.39 |
| Average | 15.0 | 20.3 | 35.2 | 52.3 | 63.7 | 72.2 | 82.7 | 96.0 | 98.1 | 98.6 | 99.8 |
| Std Dev | 0.3 | 1.1 | 2.7 | 2.5 | 2.2 | 2.1 | 2.2 | 2.3 | 2.4 | 2.5 | 2.3 |
| % RSD | 2.3 | 5.6 | 7.6 | 4.8 | 3.5 | 3.0 | 2.6 | 2.4 | 2.5 | 2.5 | 2.3 |

TABLE 36
Formulation 81

| | D-Amphetamine | % Cumulative drug released (%) | | | | | | | | | |
|---|---|---|---|---|---|---|---|---|---|---|---|
| Dissolution | Sulfate (mg) | 0.25 hr | 0.5 hr | 1 hr | 1.5 hr | 2 hr | 3 hr | 6 hr | 9 hr | 12 hr | 24 hr |
| #1 | 14.68 | 18.75 | 34.93 | 51.33 | 62.09 | 69.55 | 79.51 | 93.86 | 96.62 | 97.44 | 99.11 |
| #2 | 15.34 | 20.50 | 38.18 | 56.28 | 67.53 | 75.64 | 85.33 | 97.92 | 100.06 | 100.94 | 102.23 |
| #3 | 14.79 | 21.50 | 35.08 | 51.39 | 62.15 | 70.55 | 80.21 | 94.43 | 96.82 | 97.26 | 98.93 |
| #4 | 14.36 | 18.80 | 32.08 | 49.34 | 60.74 | 69.05 | 78.49 | 91.27 | 93.28 | 93.78 | 95.39 |
| Average | 14.8 | 20.3 | 36.1 | 53.0 | 63.9 | 71.9 | 81.7 | 95.4 | 97.8 | 98.5 | 100.1 |
| Std Dev | 0.4 | 1.4 | 1.8 | 2.8 | 3.1 | 3.3 | 3.2 | 2.2 | 1.9 | 2.1 | 1.9 |
| % RSD | 2.8 | 6.9 | 5.1 | 5.4 | 4.9 | 4.5 | 3.9 | 2.3 | 2.0 | 2.1 | 1.9 |

TABLE 37

| Formulation ID | %, Cumulative release after 3 hrs extraction |
|---|---|
| 79 | 55 |
| 80 | 54 |
| 81 | 52 |

### Example 13

### Preparation and Analysis of Additional Extended Release Oxycodone Compositions (Formulations 82-101)

Oxycodone compositions were prepared and characterized with respect to dissolution and abuse deterrence characteristics as indicated below.

### Materials and Methods

The compositions were prepared to provide the compositions indicated in Tables 38-41 below. Component amounts are % w/w relative to the total weight of the formulation including oxycodone base prior to encapsulation, unless otherwise indicated.

The placebo formulations were prepared in 500 g scale. A stock solution, SAIB/TA (1.35), was prepared before the compounding procedure started. The preparation took place in a 60° C.±5° C. water bath. SAIB/TA (1.35) was transferred into a jar, and BHT was added to the solution while mixing at 500 rpm. Then CAB was added to the solution, and mixed @1500 RPM until all the particles were dissolved. IPM was added to the mixture and dispersed uniformly, and then HEC was added into the jar and mixed for 30 minutes. Cab-o-Sil® particles were added to the mixture and were dispersed uniformly. Part of the placebo formulation was transferred into a separate jar and oxycodone base was introduced into the mixture and dispersed well to make 100 g active formulations. Active formulations were filled into size 00 gelatin capsules.

TABLE 38

| | | Formulation Composition (with 0.02% BHT) | | | | | | | |
|---|---|---|---|---|---|---|---|---|---|
| Formulation | Dose/Fill Wt | SAIB/TA | | | | | | | Cab-o-sil ® |
| ID # | (mg/mg) | Ratio | Oxycodone % | SAIB % | Triacetin (TA) % | IPM % | CAB % | HEC % | % |
| 82 | 80/780 | 1.35 | 10.26 | 35.70 | 26.45 | 15.26 | 4.71 | 5.38 | 2.24 |
| 83 | 80/780 | | | | | | | | |
| 84 | 80/780 | 1.35 | 10.26 | 35.44 | 26.26 | 15.26 | 4.71 | 5.38 | 2.69 |

TABLE 39

| | | Formulation Composition (with 0.02% BHT) | | | | | | | | |
|---|---|---|---|---|---|---|---|---|---|---|
| Formulation | Dose/Fill Wt | SAIB/TA | | | | | | | Cab-o-sil ® | |
| ID # | (mg/mg) | Ratio | Oxycodone % | SAIB % | Triacetin % | IPM % | CAB % | HEC % | % | SDS % |
| 85 | 80/780 | 1.5 | 10.26 | 37.42 | 24.95 | 14.36 | 5.38 | 5.38 | 2.24 | 0 |
| 86 | 80/780 | 1.35 | 10.26 | 36.35 | 26.92 | 13.46 | 5.38 | 5.38 | 2.24 | 0 |
| 87 | 80/780 | 1.35 | 10.26 | 36.27 | 26.87 | 14.36 | 4.49 | 5.38 | 2.24 | 0.13 |
| 88 | 80/780 | 1.35 | 10.26 | 36.71 | 27.19 | 13.46 | 4.49 | 5.38 | 2.24 | 0.27 |
| 89 | 80/780 | 1.5 | 10.26 | 37.88 | 25.25 | 13.46 | 5.38 | 5.38 | 2.24 | 0.13 |
| 90 | 80/780 | 1.5 | 10.26 | 37.80 | 25.20 | 14.36 | 4.49 | 5.38 | 2.24 | 0.27 |

TABLE 40

| | Dose/Fill | Formulation Composition (with 0.02% BHT) | | | | | | | | |
|---|---|---|---|---|---|---|---|---|---|---|
| Formulation | Wt | SAIB/TA | | | Triacetin | | | | Cab-o-sil ® | Gelucire ® |
| ID # | (mg/mg) | Ratio | Oxycodone % | SAIB % | (TA) % | IPM % | CAB % | HEC % | % | 50/13% |
| 91 | 80/780 | 1.35 | 10.26 | 36.48 | 27.02 | 14.36 | 5.38 | 2.69 | 2.02 | 1.79 |
| 92 | 80/780 | 1.35 | 10.26 | 36.48 | 27.02 | 14.36 | 5.38 | 2.69 | 2.02 | 1.79* |
| 93 | 80/780 | 1.35 | 10.26 | 36.22 | 26.83 | 14.36 | 4.94 | 5.38 | 2.02 | 0 |

*Gelucire ® 44/14

TABLE 41

| | Dose/Fill | Formulation Composition (with 0.02% BHT) | | | | | | | | |
|---|---|---|---|---|---|---|---|---|---|---|
| Formulation | Wt | SAIB/TA | | | Triacetin | | | | Cab-o-sil ® | Gelucire ® |
| ID # | (mg/mg) | Ratio | Oxycodone % | SAIB % | (TA) % | IPM % | CAB % | HEC % | % | 44/14% |
| 97 | 80/780 | 1.35 | 10.26 | 35.83 | 26.54 | 14.36 | 4.49 | 6.28 | 2.24 | 0 |
| 98 | 80/780 | 1.3 | 10.26 | 35.25 | 27.12 | 14.36 | 4.49 | 6.28 | 2.24 | 0 |
| 99 | 80/780 | 1.35 | 10.26 | 35.88 | 26.58 | 14.36 | 4.94 | 4.49 | 2.15 | 1.35 |
| 100 | 80/780 | 1.4 | 10.26 | 36.25 | 25.90 | 14.36 | 4.94 | 5.38 | 2.02 | 0.90 |
| 101 | 80/780 | 1.5 | 10.26 | 37.10 | 24.73 | 13.46 | 5.38 | 5.38 | 2.15 | 1.53 |

Dissolution Testing

Dissolution experiments were performed using 2-phase medium in a USP Apparatus 2. The capsules were placed in stainless steel (316SS) wire spiral capsule sinkers for dissolution testing. The dissolution parameters were as follows: Dissolution medium: 750 ml 0.1N HCl for the first 2 hours, add 250 ml 0.2 M phosphate buffer to achieve a final pH of 6.8; Paddle speed: 100 rpm; Vessel temperature: 37 C. Sampling time points: 0.25, 0.5, 1, 2, 3, 6, 10, 12, 18 and 24 hours. Sampling volume: 1 mL.

The HPLC parameters were as follows: Mobile phase A: 0.5% sodium dodecyl sulfate 1% glacial acetic acid, 20% acetonitrile; Mobile phase B: 100% acetonitrile; Mobile phase: 65% Mobile phase A and 35% Mobile phase B; 240 nm wavelength. Capsule number=2-4 capsules per testing.

Abuse Deterrence

Capsules from each composition were tested for abuse deterrence characteristics. The release rate of oxycodone was determined using an isocratic HPLC method at defined time points. The capsules were subjected to 60 mL of acidified 80-proof ethanol with vigorous shaking Each capsule was placed in a wide mouth round jar containing 36 mL of 0.1 N HCl and 24 mL 200-proof of ethanol. The sample jar was placed in a shaking incubator maintained at 25° C. with 240 rpm shaking speed over the course of the 3 hour extraction test. The sampling time points were 0.5, 1, and 3 hours. A 1 mL sample was taken at each time point and assayed using reverse-phase HPLC at 240 nm wavelength. The mobile phase included 0.35% (w/v) SDS/0.7% (v/v) acetic acid/44% (v/v) acetonitrile in water.

Viscosity Measurements

Samples of the above compositions were analyzed for rheological properties using an Anton Paar MCR 301 Rheometer equipped with a parallel plate (25 mm diameter) and a gap setting of 1 mm. The temperature for the measuring plate was set @25° C. The samples were exposed to increasing frequency (0.1 s⁻¹ to 100 s⁻¹) (at 5 point/° C.) at constant (0.5%) strain (oscillation mode). The zero shear viscosity of each samples were then determined by internal Carreau-Yasuda Analysis Method.

### Results

Dissolution Testing, Abuse Deterrence and Viscosity Results

The results of the dissolution, abuse deterrence and viscosity testing experiments are provided in Tables 42-45 (below).

TABLE 42

| | Dose/Fill | Dissolution (TM-254B) | | | | | | %, Abuse |
|---|---|---|---|---|---|---|---|---|
| Formulation | Wt | % | % | % | % | % | % | after 3 hrs |
| ID # | (mg/mg) | 0.5 hr | 3 hr | 6 hr | 10 hr | 12 hr | 24 hr | extraction |
| 82 | 80/780 | 7 | 25 | 45 | 74 | 91 | 100 | 34 |
| 83 | 80/780 | 8 | 29 | 53 | 85 | 101 | 109 | 36 |
| 84 | 80/780 | 7 | 24 | 40 | 68 | 85 | 95 | 44 |

TABLE 43

| | Dose/Fill | Dissolution (2 phase) | | | | | | | %, Abuse | |
|---|---|---|---|---|---|---|---|---|---|---|
| Formulation | Wt | % | % | % | % | % | % | % | after 3 hrs | zero shear |
| ID # | (mg/mg) | 0.5 hr | 2 hr | 3 hr | 6 hr | 10 hr | 12 hr | 24 hr | extraction | viscosity (Pas) |
| 85 | 80/780 | 7 | 14 | 18 | 25 | 33 | 39 | 44 | 28 | 525 |
| 86 | 80/780 | 9 | 18 | 22 | 30 | 39 | 46 | 50 | 38 | 448 |
| 87 | 80/780 | 11 | 20 | 26 | 35 | 45 | 53 | 60 | 49 | 178 |
| 88 | 80/780 | 9 | 19 | 24 | 32 | 42 | 49 | 56 | 41 | 218 |
| 89 | 80/780 | 7 | 15 | 19 | 26 | 34 | 40 | 44 | 30 | 482 |
| 90 | 80/780 | 8 | 18 | 23 | 32 | 42 | 49 | 54 | 38 | 201 |

TABLE 44

| | Dose/Fill | Dissolution (2 phases) | | | | | | | %, Abuse |
|---|---|---|---|---|---|---|---|---|---|
| Formulation | Wt | % | % | % | % | % | % | % | after 3 hrs |
| ID # | (mg/mg) | 0.5 hr | 2 hr | 3 hr | 6 hr | 10 hr | 12 hr | 24 hr | extraction |
| 91 | 80/780 | 13 | 30 | 36 | 43 | 51 | 57 | 62 | 27 |
| 92 | 80/780 | 12 | 30 | 36 | 44 | 52 | 58 | 63 | 29 |

TABLE 45

| | Dose/Fill | Dissolution (2 phases) | | | | | | | %, Abuse |
|---|---|---|---|---|---|---|---|---|---|
| Formulation | Wt | % | % | % | % | % | % | % | after 3 hrs |
| ID # | (mg/mg) | 0.5 hr | 2 hr | 3 hr | 6 hr | 10 hr | 12 hr | 24 hr | extraction |
| 97 | 80/780 | 6 | 17 | 22 | 30 | 39 | 46 | 52 | 45 |
| 98 | 80/780 | 6 | 18 | 23 | 31 | 41 | 48 | 54 | 43 |
| 99 | 80/780 | 7 | 28 | 34 | 41 | 49 | 56 | 62 | 41 |
| 100 | 80/780 | 5 | 22 | 28 | 35 | 44 | 52 | 59 | 41 |
| 101 | 80/780 | 6 | 25 | 31 | 38 | 46 | 53 | 59 | 43 |

### Example 14

### Preparation and In Vitro Analysis of Extended Release Oxymorphone Composition (Formulation 102)

An oxymorphone composition was prepared and characterized with respect to stability, abuse deterrence characteristics, and dissolution characteristics as indicated below.

### Materials and Methods

Oxymorphone Formulation 102 was prepared in 1 Kg scale for GLP study use. Sucrose Acetate Isobutyrate (SAIB) and Gelucire® 44/14 were heated in a 60° C. (±10° C.) oven at least 1 hour prior to use. The temperature of the compounding was maintained at 60° C. (±10° C.) throughout the process. Heated SAIB was first transferred into a glass jar. Triacetin was added and mixed at 600 rpm for 30 minutes until the mass became clear. Butylated hydroxytoluene (BHT) was dissolved first in isopropyl myristate (IPM) and then added into a jar and mixed for 20 minutes. Gelucire® was added, and the mixture was mixed for an additional 20 minutes. Colliodal silicon dioxide (Cab-o-Sil®) was added into the jar, and the mixing speed was increased to 800 rpm for 20 minutes. The mixture was homogenized using Fisher PowerGen 500 with a setting of 9600 rpm for 5 minutes. Sieved cellulose acetate butyrate (CAB) was added to the jar while mixing at 1500 rpm for 35 minutes. Oxymorphone hydrochloride was added and mixed for 35 minutes. Finally, sieved hydroxyethyl cellulose (HEC) was added into the jar and mixed for 20 minutes to complete the formulation. The final formulation was homogenized using Fisher PowerGen 500 with a setting of 9600 rpm for 5 minutes. The compounded bulk formulation was then filled into size #2 white opaque hard gelatin capsules with a net fill weight of 275 mg to achieve 20 mg dose strength. Twenty capsules were packaged in 40 cc white HDPE bottles.

The % w/w of the various formulation components for the active formulation and the placebo formulation (without oxymorphone) prior to encapsulation are provided below in Table 46.

TABLE 46

| Formulation component | % w/w (Active) | % w/w (Placebo) |
|---|---|---|
| Oxymorphone hydrochloride | 7.27 (% w/w), 20 mg | 0 |
| SAIB | 40.77 | 43.97 |
| Triacetin | 27.18 | 29.31 |
| IPM | 13.91 | 15.00 |
| CAB (CAB-381-20BP) | 5.10 | 5.50 |
| HEC | 2.78 | 3.00 |
| Colloidal silicon dioxide | 1.86 | 2.00 |
| Lauroyl polyoxylglycerides | 1.11 | 1.20 |
| (Gelucire ® 44/14) | | |
| BHT | 0.02 | 0.02 |

Stability

Samples of the above formulation were stored at 25° C./60% relative humidity (RH). At the one-month time point±5 days, samples were pulled for testing. The appearance of the capsule was examined visually for its color and size.

The identification, uniformity of dosage units, potency and chromatographic impurity of the OMH extended-release capsules were determined by an RP-HPLC method. The method utilized a 5 mM 1-Decanesulfonic acid sodium salt and 5 mM NaH₂PO₄ buffer, adjusted to pH 2.4 with 85% phosphoric acid, and acetonitrile gradient. The OMH was detected at 230 nm using a C₁₈, 4.6×150 mm (5 μm) HPLC column at 30° C. The positive identity of the test article was established relative to the reference standard by comparison of HPLC retention times (within 5% of each other). The % label strength was controlled at 90.0% to 110.0% per USP <905>.

Abuse Deterrence

The extraction extent of oxymorphone hydrochloride was determined from six capsules using the following procedures. Each capsule was soaked in 36 ml 0.1N HCl for 5 minutes, followed by the addition of 24 ml 200-proof ethanol to achieve a final 800-proof ethanol solution. The capsules were subjected to a shaking speed of 240 rpm and an incubation temperature of 25° C. for the remainder of the test. The standard sampling time points were 0.5, 1, and 3 hours. A 1 mL sample was taken at each time point and assayed using reverse-phase HPLC at 240 nm wavelength. The mobile phase included 0.35% (w/v) SDS/0.7% (v/v) acetic acid/40% (v/v) acetonitrile in water.

Dissolution Testing

The release rate of oxymorphone hydrochloride was determined for six capsules using a USP Apparatus 2 dissolution tester. Dissolution medium containing 1000 ml 0.1 N HCl with 0.5% (w/w) SDS was maintained at 37° C. with 100 rpm paddle speed over the course of the 24 hour dissolution test. A 20 mesh screen hanging basket was incorporated to hold the test article. The standard sampling time points were 0.5, 2, 3, 6, 12, 18 and 24 hours. A 1 mL sample was taken at each time point and assayed using reverse-phase HPLC at 240 nm wavelength. The mobile phase included 0.35% (w/v) SDS/0.7% (v/v) acetic acid/40% (v/v) acetonitrile in water.

### Results

Stability

The stability results are provided in Tables 47 and 48 below.

TABLE 47
Appearance, Identity, Uniformity of Dosage Units and Potency of
Formulation 102

| | | | | Oxymorphone | |
|---|---|---|---|---|---|
| | | | | Hydrochloride | |
| | | | | Content | Potency (% |
| Time, | Storage | | | Uniformity (% | Label |
| month | Condition | Appearance | Identification | Label Strength) | Strength) |
| 0 | Initial | Size 0 | HPLC retention | 106.4 | |
| | | capsules, | time is the same | 99.2 | |
| | | white | as the reference | 104.7 | |
| | | opaque | standard for | 104.7 | |
| | | (body)/white | oxymorphone | 99.5 | |
| | | opaque | hydrochloride | 103.1 | |
| | | (cap) with | | 103.6 | |
| | | no ink bars | | 98.6 | |
| | | | | 99.5 | |
| | | | | 104.7 | |
| | | | | Ave: 102.4 | 101.8³ |
| | | | | Acceptance Value | 102.1³ |
| | | | | (AV)²: 7.9 | |
| | | | Average | | 102.0 |
| 1 | 25° C./60% | Size 0 | | | 103.5³ |
| | RH | capsules, | | | 98.7³ |
| | | white | Average | | 101.1 |
| | | opaque | | | |
| | | (body)/white | | | |
| | | opaque | | | |
| | | (cap) with | | | |
| | | no ink bars | | | |

¹RH: relative humidity
²The acceptance value (AV) passed the USP maximum allowed value of L1 = 15.0
³Composite assay of 5 capsules.

The appearance of the OMH capsules did not change over the testing period. In addition, the above results show that the capsules produced were uniform throughout the filling process. The OMH potency results for the 25° C./60% RH storage conditions at 1 month remained within the assay specifications of 90.0% to 110.0% label strength, and indicated that the test articles were adequately chemically stable during their use in the in-vivo study discussed below.

TABLE 48
Degradation products

| Time Point | | | Individual | Total |
|---|---|---|---|---|
| (month) | Storage Condition | | Degradants | Degradants |
| 0 | Initial | Potency | No individual | ≦0.1% |
| | | | degradant greater | |
| | | | than 0.1% | |
| 1 | 25° C./60% | Potency | No individual | ≦0.1% |
| | RH | | degradant greater | |
| | | | than 0.1% | |

As shown above, no individual degradant greater than 0.1% was found at the initial and one month time points.

Abuse Deterrence

The cumulative % of oxymorphone released from each composition at sampling time points 0.5, 1, and 3 hours as determined by reverse-phase HPLC is provided in Table 49 below.

TABLE 49

| | Time (hrs) | | |
|---|---|---|---|
| | 0.5 | 1 | 3 |
| #1 | 3 | 4 | 10 |
| #2 | 3 | 4 | 9 |
| #3 | 3 | 5 | 9 |
| #4 | 3 | 5 | 10 |
| #5 | 3 | 4 | 9 |
| #6 | 3 | 4 | 10 |
| Average | 3 | 4 | 10 |
| Std Dev | 0 | 1 | 1 |
| % RSD | 0 | 25 | 10 |

Dissolution Testing

The results of the dissolution testing experiment are provided below in Table 50.

TABLE 50

| Time | Storage Condition | Dissolution Time (hr) | | | | | | |
|---|---|---|---|---|---|---|---|---|
| Month | (Test Start Date) | 0.5 | 2.0 | 3.0 | 6.0 | 12.0 | 18.0 | 24.0 |
| 0 | Initial | 6 | 21 | 29 | 45 | 66 | 81 | 89 |
| | | 6 | 21 | 27 | 43 | 64 | 78 | 88 |
| | | 6 | 21 | 29 | 47 | 69 | 83 | 91 |
| | | 7 | 21 | 28 | 45 | 69 | 85 | 96 |
| | | 7 | 23 | 31 | 48 | 71 | 86 | 96 |
| | | 6 | 21 | 28 | 45 | 68 | 83 | 93 |
| | Average | 6 | 21 | 29 | 46 | 68 | 83 | 92 |
| | Relative Standard Deviation | 17 | 5 | 3 | 4 | 3 | 4 | 3 |
| | Min | 6 | 21 | 27 | 43 | 64 | 78 | 88 |
| | Max | 7 | 23 | 31 | 48 | 71 | 86 | 96 |
| 1 | 25° C./60% RH | 5 | 17 | 24 | 39 | 62 | 79 | 90 |
| | | 5 | 21 | 28 | 46 | 70 | 84 | 92 |
| | | NA* | 17 | 24 | 40 | 60 | 74 | 84 |
| | | 6 | 19 | 25 | 41 | 61 | 77 | 89 |
| | | 5 | 17 | 23 | 38 | 60 | 77 | 89 |
| | | 5 | 17 | 24 | 41 | 65 | 80 | 89 |
| | Average | 5 | 18 | 25 | 41 | 63 | 79 | 89 |
| | Relative Standard Deviation | 0 | 11 | 8 | 7 | 6 | 4 | 3 |
| | Min | 5 | 17 | 23 | 38 | 60 | 74 | 84 |
| | Max | 6 | 21 | 28 | 46 | 70 | 84 | 92 |

| | | | | | | | |
|---|---|---|---|---|---|---|---|
| Specification | Record | NMT 40 | Record | 25-70 | 45-90 | Record | NLT70 |
| Passes Spec? | NA | Pass | NA | Pass | NA | NA | Pass |

*There was no sample taken for #3 at the 0.5 hr time point due to autosampler sampling error. This has been taken into account for the final results.

The in-vitro drug dissolution testing results showed that there was no dumping or bursting effect for both initial and one-month stability samples. The data shows that there is no significant change in dissolution rate after storage.

### Example 15

### In Vivo Analysis of Extended Release Oxymorphone Compositions (Formulation 102)

Oxymorphone formulations for in vivo testing were prepared as discussed above in Example 14 and evaluated to determine the safety, pharmacokinetic profile and relative bioavailability when administered as oral capsule doses in dogs over five days.

### Materials and Methods

Animal Acquisition and Acclimation

A total of 17 male experimentally naïve beagle dogs, approximately 5.5 to 6.5 months at receipt, were received from supplier. During the 10 day acclimation period, the animals were observed daily with respect to general health and any signs of disease. Ova and parasite evaluations on stool samples were performed, and all results were negative for animals placed on study.

Randomization, Assignment to Study, and Maintenance

The animals considered suitable for study were weighed. Using a standard, by weight, measured value randomization procedure, 15 male animals (weighing 9.35 to 10.40 kg at randomization) were assigned to the control and treatment groups identified in Table 51 below.

TABLE 51
Group Assignments

| | | | Dose Level | Number of |
|---|---|---|---|---|
| Group | | Route of | (mg/animal/ | Animals |
| Number | Formulation | Administration | dose)^{a,b} | Male |
| 1 | negative control | Oral Capsule | 0 | 5 |
| | (placebo 102 | | | |
| | formulation) | | | |
| 2 | positive control | Oral Tablet | 20 | 5 |
| | (Opana ® ER) | | | |
| 3 | 102 | Oral Capsule | 20 | 5 |

^{a}Each animal was dosed twice daily for four days, approximately 12 hours apart. On Day 5, animals were dosed once.
^{b}Each animal received approximately 25 mg of Naltrexone orally via tablets approximately 30 minutes prior to each dose.

Animals assigned to study had body weights within ±20% of the mean body weight. Extra animals obtained for the study, but not placed on study, were transferred to the stock colony. Each animal was assigned an animal number to be used in the Provantis™ data collection system and was implanted with a microchip bearing a unique identification number. Each dog was also identified by a permanent tattoo of a vendor animal number on an earflap. The individual animal number, implant number, and study number comprised a unique identification for each animal. Each cage was identified by the animal number, study number, group number, and sex. Animal identification was verified during the course of the study, as documented in the data.

The dogs were housed individually in single-sized stainless steel suspended cages with plastic coated flooring in an environmentally controlled room. The dogs were provided the opportunity for exercise for at least 30 minutes, twice during the study, according to SOP. Fluorescent lighting was provided for approximately 12 hours per day. The dark cycle was interrupted intermittently due to study-related activities. Temperature and humidity were continuously monitored, recorded, and maintained to the maximum extent possible within the protocol-designated ranges of 64 to 84° F. and 30 to 70%, respectively.

Block Lab Diet® (Certified Canine Diet #5007, PMI Nutrition International, Inc.) was available ad libitum, except during designated periods. The lot number from each diet lot used for this study was recorded. Certification analysis of each diet lot was performed by the manufacturer. Tap water was available ad libitum via an automatic watering system. On one occasion, animals were offered ice cubes made from the automatic watering system. The water supply was monitored for specified contaminants at periodic intervals according to SOP.

### Administration

Approximately 30 minutes (±5 minutes) prior to test or control article administration, each animal received approximately 25 mg of naltrexone via oral tablet (50 mg tablet cut in half). Each tablet dose was immediately followed by administration of approximately 15 mL of deionized water. The control and test articles were administered twice a day on Days 1 through 4 and once on Day 5 oral via gelatin capsule or tablet. The dose levels were 0, 20, and 20 mg/animal/dose. Each dose was immediately followed be administration of approximately 10 mL of deionized water.

### Plasma Analysis

Blood samples (approximately 2 mL) were collected from all animals via the jugular vein for determination of the plasma concentrations of the test article. Samples were collected on Day 1 prior to dosing and at 10, 20, 40 minutes, 1, 1.5, 2, 3, 4, 6, 8, and 12 hours after the first dose; on Days 2 to 4 prior to the first dose; and on Day 5 prior to dosing and at 10, 20, 40 minutes, 1, 1.5, 2, 3, 4, 6, 8, 12, 16, 24, and 48 hours postdose. The animals were not fasted prior to blood collection. Samples were placed in tubes containing K₂EDTA anticoagulant. The samples were collected on wet ice and maintained on an ice block throughout processing until centrifuged. \Plasma samples were contained in tightly capped, pre-labeled, plastic vials. Samples collected predose and at 10, 20, and 40 minutes postdose on Day 1 were initially stored on dry ice when necessary then stored frozen at −50 to −90° C. after the one hour postdose sample collection until shipped to an analysis lab for analysis of plasma concentrations of the test article. The vial label included the study number, relative study day, animal number, and the date and time interval of collection.

Pharmacokinetic Analysis

The pharmacokinetic (PK) parameters were determined for oxymorphone and, if appropriate, its metabolites, from individual animal concentration-time data in the test species. Plasma samples were analyzed for oxymorphone, 6β-hydroxyoxymorphone and oxymorphone-glucuronide. The assay ranges were 0.0500 to 50.0 ng/mL for oxymorphone, 0.0200 to 20.0 ng/mL for 6β-hydroxyoxymorphone, and 1.00 to 1000 ng/mL for oxymorphone-glucuronide in beagle K2-EDTA plasma.

The following pharmacokinetic parameters were calculated for oxymorphone, 6β-hydroxyoxymorphone, and oxymorphone-glucuronide (providing sufficient quantifiable concentration-time data were available).

Cₘₐₓ: The maximum drug concentration in plasma determined directly from individual concentration-time data; reported to 3 significant figures.

Tₘₐₓ: Time to reach maximum concentration; reported to 2 decimal places.

Cₗₐₛₜ: The last quantifiable drug concentration determined directly from individual concentration-time data; reported to 3 significant figures.

Tₗₐₛₜ: Time of the last quantifiable concentration; reported to 2 decimal places.

AUC₀₋₁₂: The area under the plasma concentration-time curve from time-zero through the first 12-hour dosing interval; calculated using the linear trapezoidal rule; reported to 4 significant figures (Days 1 and 5).

AUC₀₋₂₄: The area under the plasma concentration-time curve from time-zero through 24 hours post-dose; calculated using the linear trapezoidal rule; reported to 4 significant figures (Day 5 only).

AUC₀₋₄₈: The area under the plasma concentration-time curve from time-zero through 48 hours post-dose; calculated using the linear trapezoidal rule; reported to 4 significant figures (Day 5 only).

AUCₗₐₛₜ: The area under the plasma concentration-time curve from time-zero to the time of the last quantifiable concentration; calculated using the linear trapezoidal rule; reported to 4 significant figures (Days 1 and 5). Note: If quantifiable data are observed through the sampling interval (12 hours on Day 1, 48 hours on Day 5), AUClast is equivalent to AUC0-12 (Day 1) and/or AUC0-48 (Day 5) and may not be tabulated separately.)

λ_{z}*: The observed elimination rate constant; estimated by linear regression through at least three data points in the terminal phase of the log concentration-time profile; reported to 4 decimal places (Day 5 only, oxymorphone).

T_{1/2}*The observed terminal elimination half-life calculated as: T_{1/2}=ln(2)/λ_{z}; reported to 2 decimal places (Day 5 only, oxymorphone).

AUC_{inf}*: Area under the concentration-time curve from time-zero extrapolated to infinity, calculated as: AUC_{inf}=AUCₗₐₛₜ+Cₗₐₛₜ/λ_{z}; reported to 4 significant figures (Day 5 only, oxymorphone).

AUC_{Extrap} (%)*The percentage of AUC_{inf} based on extrapolation; reported to 2 decimal places (Day 5 only, oxymorphone).

*Due to the difficulty in estimating and interpreting the observed terminal elimination phase of the metabolites, the observed elimination rate constant and parameters based on extrapolation using the elimination rate constant were reported only for oxymorphone. However, if quantifiable concentration-time data were not observed over the sampling period, extrapolation was accepted for estimating partial AUCs (AUC₀₋₁₂, AUC₀₋₂₄, AUC₀₋₄₈).

Relative Bioavailability

The bioavailability of oxymorphone from Formulation 102 relative to Opana ER (Fᵣₑₗ) was determined using the mean AUC₀₋₁₂ on Days 1 and 5 (separately), according to the following equation, Fᵣₑₗ (%)=100*[AUC₀₋₁₂ (Formulation 102)]/[AUC₀₋₁₂ (Opana ER)]. (Days 1 and 5) In addition, the relative bioavailability was estimated using AUC₀₋₄₈ (or AUCₗₐₛₜ) on Day 5, Fᵣₑₗ (%)=100*[AUC₀₋₄₈ (Formulation 102)]/[AUC₀₋₄₈ (Opana ER)]. (Day 5 only) Analogous percent ratios were calculated for 6β-hydroxyoxymorphone and oxymorphone-glucuronide.

Accumulation

The accumulation of oxymorphone, 6β-hydroxyoxymorphone, and oxymorphone-glucuronide during BID administration of Formulation 102 and Opana ER was assessed using the mean AUC₀₋₁₂ on Days 1 and 5. For each treatment, accumulation was calculated as follows: R=AUC₀₋₁₂ (Day 5)/AUC₀₋₁₂ (Day 1).

### Results

Concentration-Time Data

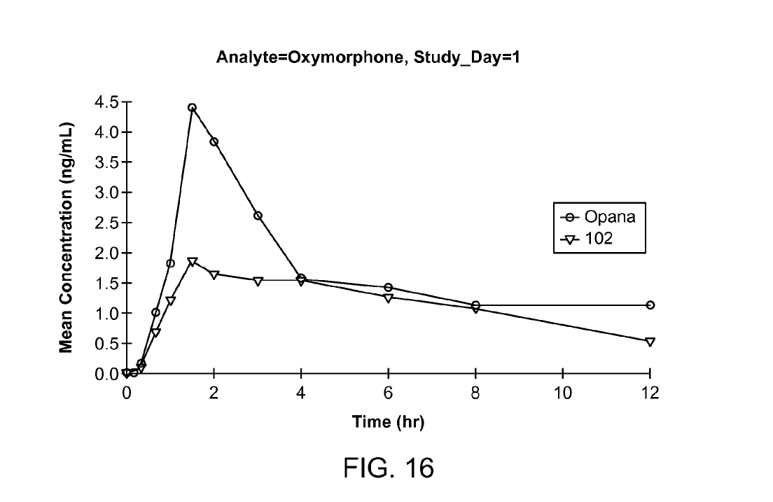

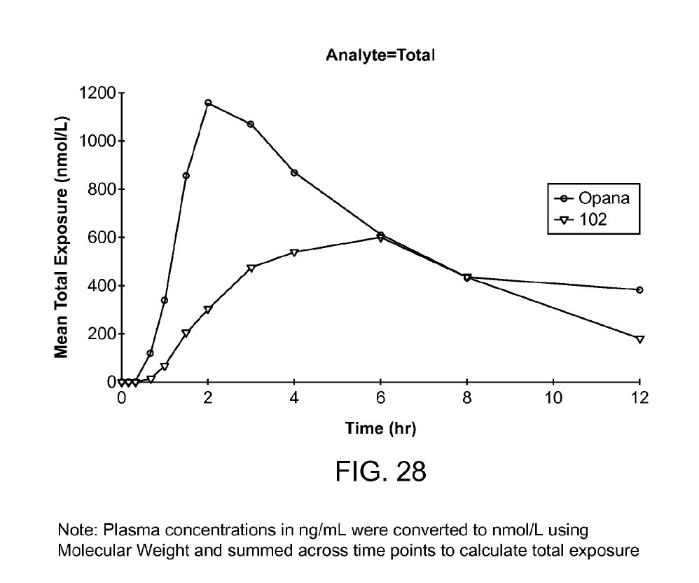

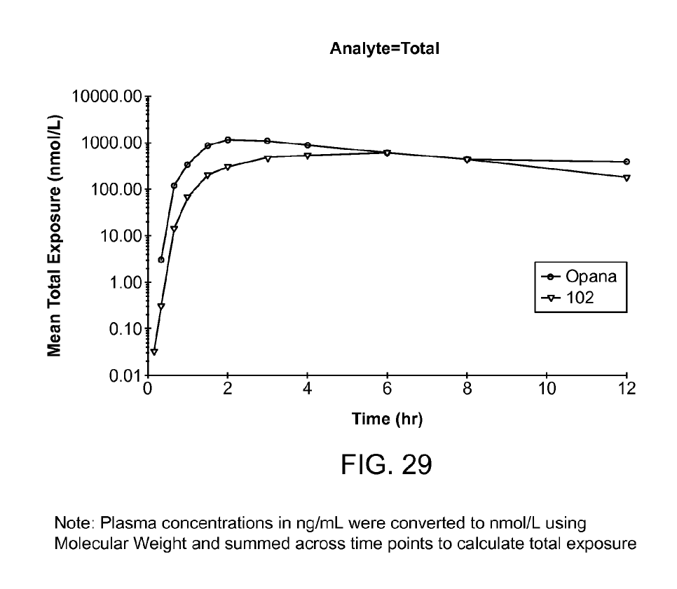

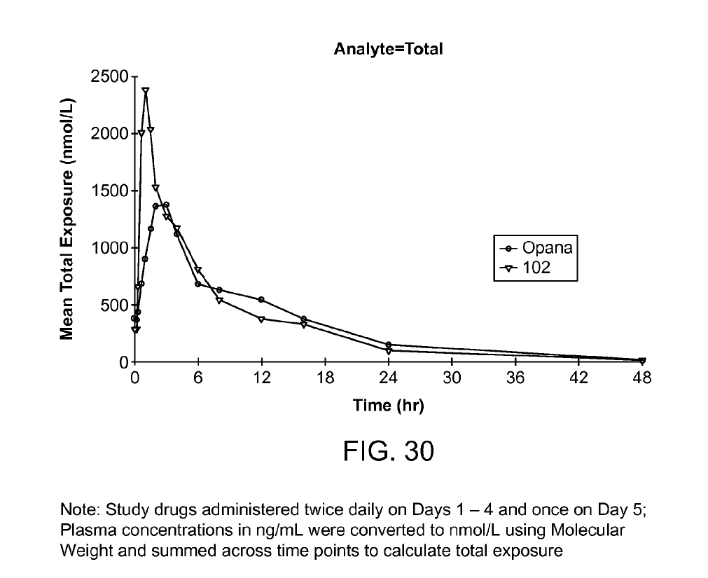

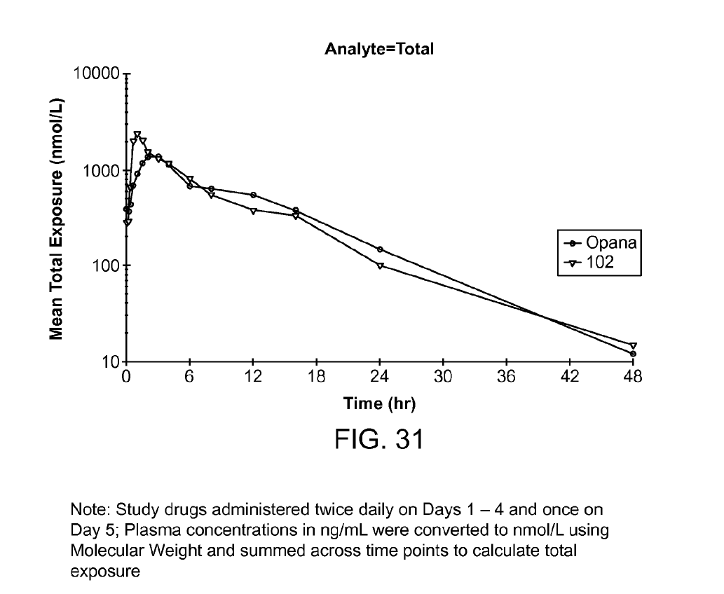

Concentration-time data are summarized in Tables 52-62 below and FIGS. 16-27. For oxymorphone, 6β-hydroxyoxymorphone, and oxymorphone-glucuronide. Total exposure data (in nmol/L) are shown in Tables 61 and 62 and FIGS. 28-31. All dogs receiving active treatment (Formulation 102 or Opana ER) exhibited exposure to the parent drug oxymorphone and its metabolites, 6β-hydroxyoxymorphone and oxymorphone-glucuronide.

Oxymorphone:

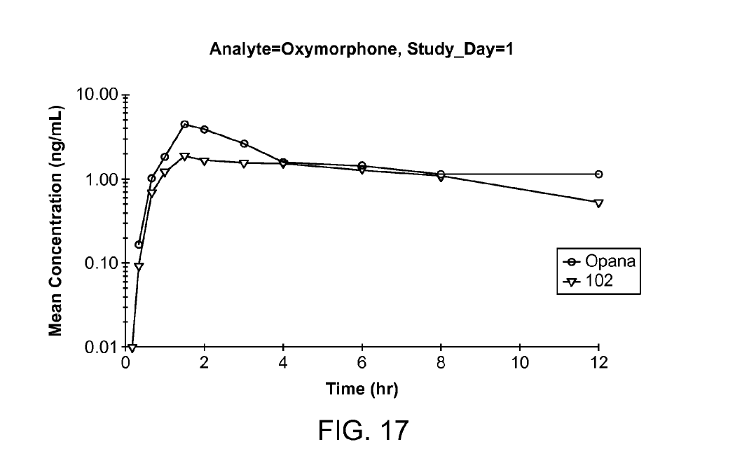

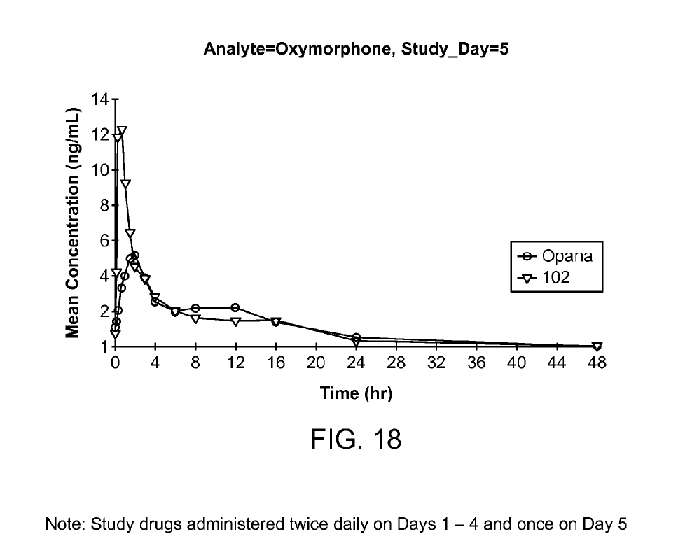

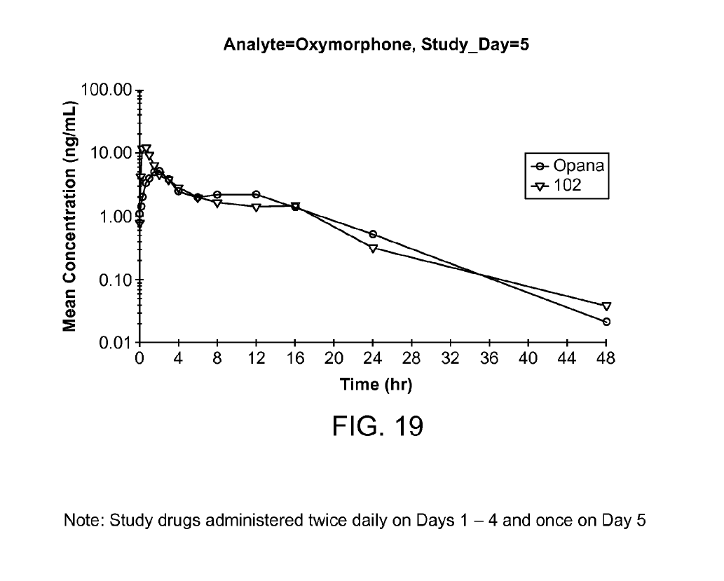

As shown in Table 52 and FIGS. 16 and 17, the first quantifiable oxymorphone concentrations after the administration of Formulation 102 on Day 1 were observed between 0.17 and 0.67 hr. The peak mean oxymorphone concentration on Day 1 was 1.86 ng/mL at 1.5 hr after Formulation 102. Variability in the oxymorphone concentration-time data after administration of Formulation 102 on Day 1 was moderate to very high, as illustrated by the CV % of 34.19% (8.00 hr) to 223.61% (0.17 hr). As shown in Table 53 and FIGS. 18 and 19, the peak mean oxymorphone concentration on Day 5 was 12.2 ng/mL at 0.67 hr after Formulation 102, over 6 times higher than the peak mean oxymorphone concentration on Day 1. Quantifiable oxymorphone concentrations were observed for most dogs (3 of 5) in the Formulation 102 treatment group throughout the 48-hour sampling interval on Day 5. Variability in the oxymorphone concentration-time data after administration of Formulation 102 on Day 5 ranged from 17.64% (2.00 hr) to 93.66% (48.00 hr). As shown in Table 54 (also Table 53 for Day 5 data), quantifiable oxymorphone concentrations were observed in the pre-dose samples on Days 2, 3, 4, and 5 and mean oxymorphone concentrations on these days ranged from 0.669 ng/mL (Day 3) to 1.08 ng/mL (Day 4). Due to the lack of continuing increases in predose oxymorphone concentrations on successive dosing days, steady-state conditions appear to have been achieved during the five day twice-daily dosing regimen of Formulation 102.

The first quantifiable oxymorphone concentrations after the administration of Opana ER on Day 1 were observed at 0.33 hr for all dogs. The peak mean oxymorphone concentration on Day 1 was 4.40 ng/mL at 1.50 hr after Opana ER. There was an unexpected increase in oxymorphone concentration for some animals receiving Opana ER, which resulted in a slight increase in the mean oxymorphone concentration at 12.00 hr. It should be noted that all 12-hour samples were taken from animals prior to dosing and, therefore, the increase in oxymorphone concentration is not explainable. Variability in the oxymorphone concentration-time data after administration of Opana ER on Day 1 was moderate to high, as illustrated by the CV % of 35.46% (8.00 hr) to 76.31% (0.33 hr). The peak mean oxymorphone concentration on Day 5 was 5.17 ng/mL at 2.00 hr after Opana ER, only slightly higher than the mean oxymorphone concentration on Day 1. Quantifiable oxymorphone concentrations were observed for some dogs (2 of 5) in the Opana ER treatment group throughout the 48-hour sampling interval on Day 5. Variability in the oxymorphone concentration-time data after administration of Opana ER on Day 5 ranged from 16.81% (6.00 hr) to 137.04% (48.00 hr). As shown in Table 54 (also Table 53 for Day 5 data), quantifiable oxymorphone concentrations were observed in the pre-dose samples on Days 2, 3, 4, and 5 and mean oxymorphone concentrations on these days ranged from 0.673 ng/mL (Day 3) to 1.48 ng/mL (Day 2). Due to the lack of increase in predose oxymorphone concentrations on successive dosing days, steady-state conditions appear to have been achieved during the twice-daily dosing regimen of Opana ER.

6β-Hydroxyoxymorphone:

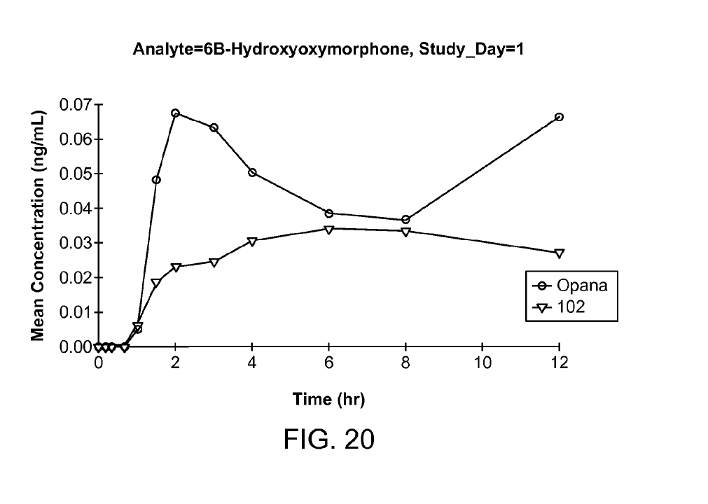

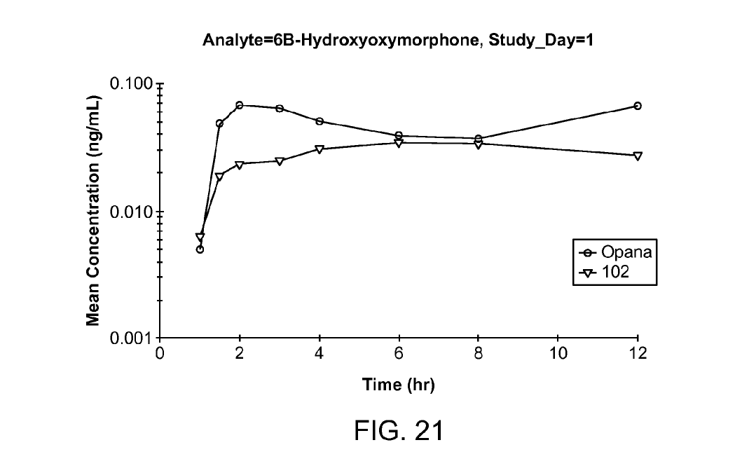

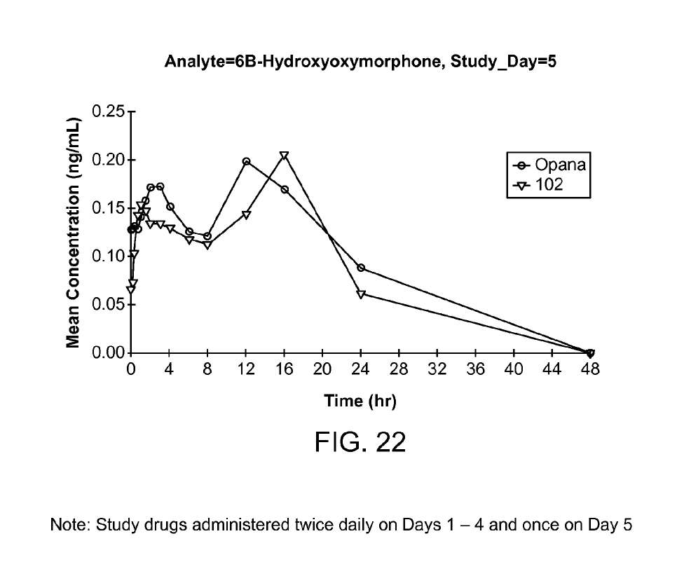

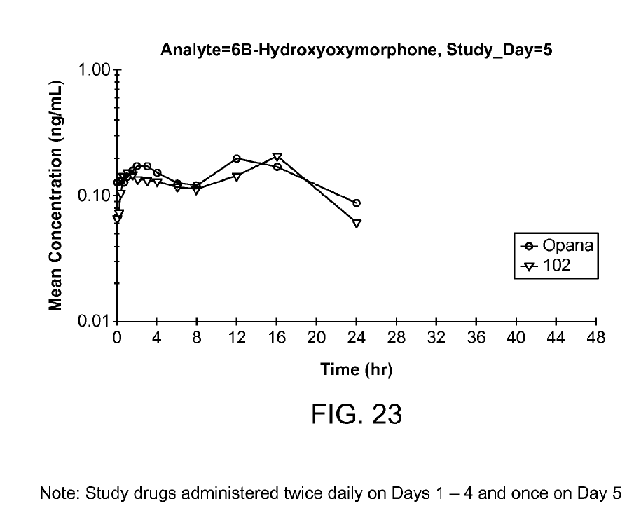

As shown in Table 55 and FIGS. 20 and 21, the first quantifiable 6β-hydroxyoxymorphone concentrations after the administration of Formulation 102 on Day 1 were observed between 1.00 and 6.00 hr. The peak mean 6β-hydroxyoxymorphone concentration on Day 1 was 0.0342 ng/mL at 6.00 hr after Formulation 102. Variability in the 6β-hydroxyoxymorphone concentration-time data after administration of Formulation 102 on Day 1 was moderate to very high, as illustrated by the CV % of 25.95% (8.00 hr) to 223.61% (1.00 hr). As shown in Table 56 and FIGS. 22 and 23, the peak mean 6β-hydroxyoxymorphone concentration on Day 5 was 0.206 ng/mL at 16.00 hr after Formulation 102. Quantifiable 6β-hydroxyoxymorphone concentrations were observed in the Formulation 102 treatment group through 24 hours postdose on Day 5. Variability in the 6β-hydroxyoxymorphone concentration-time data after administration of Formulation 102 on Day 5 ranged from 30.28% (0.17 hr) to 88.12% (16.00 hr). As shown in Table 57 (also Table 56 for Day 5 data), quantifiable 6β-hydroxyoxymorphone concentrations were observed in the pre-dose samples on Days 2, 3, 4, and 5 and mean 6β-hydroxyoxymorphone concentrations on these days ranged from 0.0493 ng/mL (Day 3) to 0.0670 ng/mL (Day 4). Due to the lack of increase in predose 6β-hydroxyoxymorphone concentrations on successive dosing days, steadystate conditions appear to have been achieved during the twice-daily dosing regimen of Formulation 102.

The first quantifiable 6β-hydroxyoxymorphone concentrations after the administration of Opana ER on Day 1 were observed between 1.00 and 1.50 hr. The peak mean 6β-hydroxyoxymorphone concentration on Day 1 was 0.0675 ng/mL at 2.00 hr after Opana ER. As noted for oxymorphone, there was an anomalous increase in 6β-hydroxyoxymorphone concentrations for some animals receiving Opana ER at 12 hours postdose. Variability in the 6β-hydroxyoxymorphone concentration-time data after administration of Opana ER on Day 1 was moderate to very high, as illustrated by the CV % of 24.35% (6.00 hr) to 223.61% (1.00 hr). The peak mean 6β-hydroxyoxymorphone concentration on Day 5 was 0.198 ng/mL at 12.00 hr after Opana ER. Quantifiable 6β-hydroxyoxymorphone concentrations were observed in the Opana ER treatment group through 24 hours postdose on Day 5. Variability in the 6β-hydroxyoxymorphone concentration-time data after administration of Formulation 102 on Day 5 ranged from 17.18% (6.00 hr) to 66.06% (24.00 hr). As shown in Table 57 (also Table 56 for Day 5 data), quantifiable 6β-hydroxyoxymorphone concentrations were observed in the pre-dose samples on Days 2, 3, 4, and 5 and mean 6β-hydroxyoxymorphone concentrations on these days ranged from 0.0668 ng/mL (Day 3) to 0.176 ng/mL (Day 4). Due to the lack of increase in predose 6β-hydroxyoxymorphone concentrations on successive dosing days, steady-state conditions appear to have been achieved during the twice-daily dosing regimen of Formulation 102.

Oxymorphone-Glucuronide:

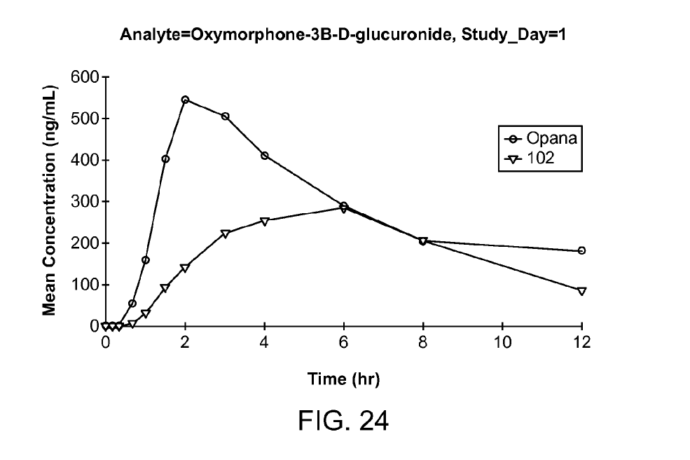

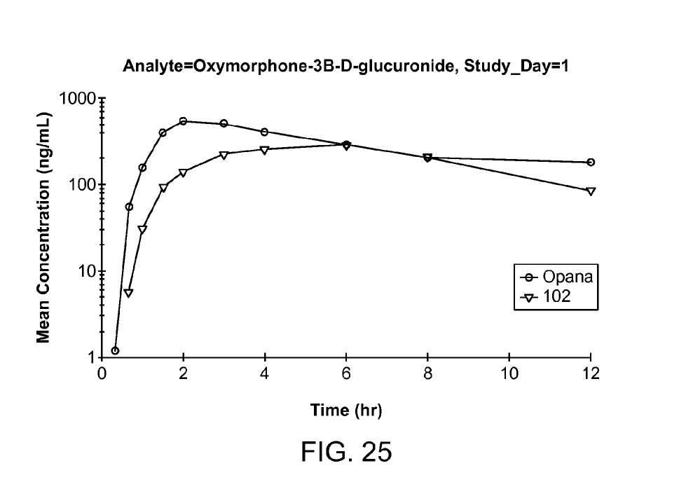

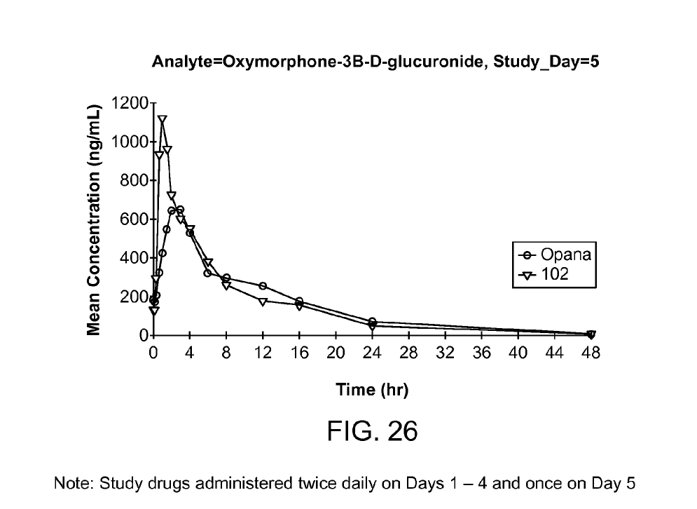

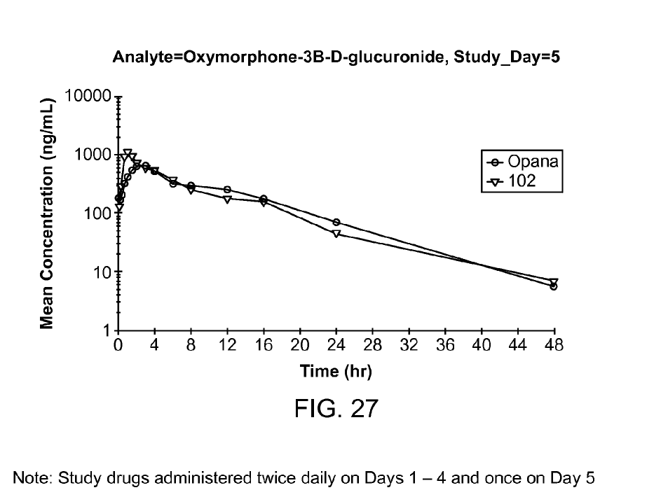

As shown in Table 58 and FIGS. 24 and 25, the first quantifiable oxymorphone-glucuronide concentrations after the administration of Formulation 102 on Day 1 were observed at 0.67 hr for all dogs. The peak mean oxymorphone-glucuronide concentration on Day 1 was 285 ng/mL at 6.00 hr after Formulation 102. Variability in the oxymorphone-glucuronide concentration-time data after administration of Formulation 102 on Day 1 was moderate to high, as illustrated by the CV % of 47.35% (12.00 hr) to 97.62% (1.00 hr). As shown in Table 59 and FIGS. 26 and 27, the peak mean oxymorphone-glucuronide concentration on Day 5 was 1120 ng/mL at 1.00 hr after Formulation 102. Quantifiable oxymorphoneglucuronide concentrations were observed in the Formulation 102 treatment group throughout the 48-hour sampling interval on Day 5. Variability in the 6β-hydroxyoxymorphone concentration time data after administration of Formulation 102 on Day 5 ranged from 10.43% (6.00 hr) to 67.20% (0.67 hr). As shown in Table 60 (also Table 59 for Day 5 data), quantifiable oxymorphone-glucuronide concentrations were observed in the pre-dose samples on Days 2, 3, 4, and 5 and mean oxymorphoneglucuronide concentrations on these days ranged from 127 ng/mL (Day 2) to 184 ng/mL (Day 4). Due to the lack of increase in predose oxymorphone-glucuronide concentrations on successive dosing days, steady-state conditions appear to have been achieved during the twice-daily dosing regimen of Formulation 102.

The first quantifiable oxymorphone-glucuronide concentrations after the administration of Opana ER on Day 1 were observed between 0.33 and 0.67 hr. The peak mean oxymorphoneglucuronide concentration on Day 1 was 546 ng/mL at 2.00 hr after Opana ER. Variability in the oxymorphone-glucuronide concentration-time data after administration of Opana ER on Day 1 was moderate to high, as illustrated by the CV % of 25.03% (8.00 hr) to 139.88% (0.33 hr). Although not as pronounced as the increase for the other analytes, there was an anomalous increase in oxymorphone-glucuronide concentration at 12.00 hr for some animals receiving Opana ER. The peak mean oxymorphone-glucuronide concentration on Day 5 was 649 ng/mL at 3.00 hr after Opana ER. Quantifiable oxymorphone-glucuronide concentrations were observed in the Opana ER treatment group throughout the 48-hour sampling interval on Day 5. Variability in the 6β-hydroxyoxymorphone concentration-time data after administration of Opana ER on Day 5 ranged from 27.21% (6.00 hr) to 69.92% (1.50 hr). As shown in Table 60 (also Table 59 for Day 5 data), quantifiable oxymorphone-glucuronide concentrations were observed in the pre-dose samples on Days 2, 3, 4, and 5 and mean oxymorphone-glucuronide concentrations on these days ranged from 149 ng/mL (Day 3) to 228 ng/mL (Day 2). Due to the lack of increase in predose oxymorphoneglucuronide concentrations on successive dosing days, steady-state conditions appear to have been achieved during the twice-daily dosing regimen of Opana ER.

Total Exposure:

As exposure to the parent drug, oxymorphone, represents only a small fraction of the total drug exposure, combined data for oxymorphone and its two major metabolites, 6β-hydroxyoxymorphone and oxymorphone-glucuronide, provide a more accurate assessment of total drug exposure. As shown in Tables 61 and 62 and FIGS. 28-31, trends in the total exposure profiles reflect those noted for oxymorphone-glucuronide, which would be expected, as oxymorphone-glucuronide concentrations were much higher than those for both oxymorphone and 6β-hydroxyoxymorphone. After administration of Formulation 102, peak mean total exposure of 601.8 nmol/L was observed at 6.00 hr on Day 1 and 2380 nmol/L at 1.00 hr on Day 5. After administration of Opana ER, peak mean total exposure of 1158 nmol/L was observed at 2.00 hr on Day 1 and 1374 nmol/L at 3.00 hr on Day 5.

TABLE 52
Day 1 Oxymorphone Concentration-Time Data after
Administration of the Control Article Opana ER 20
mg and the Test Article Formulation 102 20 mg.

| | Control Article | | | | Test Article | | | |
|---|---|---|---|---|---|---|---|---|
| | Opana ER 20 mg | | | | Formulation | | | |
| Time | | Mean | SD | CV | 102 20 mg | | | |
| (hr) | n | (ng/mL) | (ng/mL) | (%) | | Mean | SD | CV |
| 0.00 | 5 | 0.00 | 0.00 | NC | 5 | 0.00 | 0.00 | NC |
| 0.17 | 5 | 0.00 | 0.00 | NC | 5 | 0.0101 | 0.0225 | 223.61 |
| 0.33 | 5 | 0.164 | 0.125 | 76.31 | 5 | 0.0929 | 0.181 | 194.90 |
| 0.67 | 5 | 1.01 | 0.570 | 56.61 | 5 | 0.687 | 0.686 | 99.84 |
| 1.00 | 5 | 1.82 | 1.14 | 62.91 | 5 | 1.21 | 1.14 | 93.73 |
| 1.50 | 5 | 4.40 | 2.79 | 63.36 | 5 | 1.86 | 1.27 | 68.20 |
| 2.00 | 5 | 3.85 | 2.24 | 58.28 | 5 | 1.65 | 1.22 | 73.68 |
| 3.00 | 5 | 2.61 | 1.15 | 44.15 | 5 | 1.54 | 1.44 | 93.68 |
| 4.00 | 5 | 1.57 | 0.672 | 42.74 | 5 | 1.53 | 1.55 | 101.08 |
| 6.00 | 5 | 1.42 | 0.671 | 47.32 | 5 | 1.26 | 0.710 | 56.43 |
| 8.00 | 5 | 1.12 | 0.399 | 35.46 | 5 | 1.07 | 0.367 | 34.19 |
| 12.00 | 5 | 1.13 | 0.641 | 56.72 | 5 | 0.525 | 0.291 | 55.38 |

Note:
Plasma samples analyzed using a bioanalytical method with a range 0.0500 to 50.0 ng/mL;
concentrations reported in ng/mL to 3 significant figures;
concentrations below limit of quantification set to zero (0.00 ng/mL) in the data summarization
NC = Not calculated

TABLE 53
Day 5 Oxymorphone Concentration-Time Data after Administration
of the Control Article Opana 20 ER mg and the Test Article
Formulation 102 20 mg Twice Daily.

| | Control Article | | | | Test Article | | | |
|---|---|---|---|---|---|---|---|---|
| | Opana ER 20 mg BID* | | | | Formulation | | | |
| Time | | Mean | SD | CV | 102 20 mg BID* | | | |
| (hr) | n | (ng/mL) | (ng/mL) | (%) | | Mean | SD | CV |
| 0.00 | 5 | 1.08 | 0.668 | 61.90 | 5 | 0.783 | 0.302 | 38.63 |
| 0.17 | 5 | 1.41 | 0.682 | 48.37 | 5 | 4.25 | 3.10 | 73.08 |
| 0.33 | 5 | 2.05 | 1.43 | 69.85 | 5 | 11.8 | 8.55 | 72.19 |
| 0.67 | 5 | 3.32 | 3.15 | 94.83 | 5 | 12.2 | 8.19 | 66.98 |
| 1.00 | 5 | 3.98 | 3.84 | 96.57 | 5 | 9.25 | 5.42 | 58.57 |
| 1.50 | 5 | 4.95 | 2.80 | 56.61 | 5 | 6.46 | 2.66 | 41.14 |
| 2.00 | 5 | 5.17 | 3.37 | 65.20 | 5 | 4.51 | 0.795 | 17.64 |
| 3.00 | 5 | 3.89 | 1.76 | 45.29 | 5 | 3.82 | 1.46 | 38.13 |
| 4.00 | 5 | 2.52 | 1.41 | 55.81 | 5 | 2.81 | 0.743 | 26.41 |
| 6.00 | 5 | 1.97 | 0.331 | 16.81 | 5 | 2.02 | 0.494 | 24.42 |
| 8.00 | 5 | 2.17 | 0.929 | 42.85 | 5 | 1.63 | 0.412 | 25.31 |
| 12.00 | 5 | 2.19 | 0.729 | 33.24 | 5 | 1.42 | 0.491 | 34.55 |
| 16.00 | 5 | 1.39 | 0.608 | 43.59 | 5 | 1.49 | 1.32 | 88.50 |
| 24.00 | 5 | 0.516 | 0.320 | 62.03 | 5 | 0.318 | 0.180 | 56.80 |
| 48.00 | 5 | 0.0211 | 0.0290 | 137.04 | 5 | 0.0386 | 0.0361 | 93.66 |

*Study drugs administered twice daily on Days 1-4 and once on Day 5
Note:
Plasma samples analyzed using a bioanalytical method with a range 0.0500 to 50.0 ng/mL;
concentrations reported in ng/mL to 3 significant figures;
concentrations below limit of quantification set to zero (0.00 ng/mL) in the data summarization

TABLE 54
Trough Oxymorphone Concentration-Time Data on Days 2, 3, and
4 after Administration of the Control Article Opana ER 20 mg
and the Test Article Formulation 102 20 mg Twice Daily

| | Control Article | | | | Test Article | | | |
|---|---|---|---|---|---|---|---|---|
| | Opana ER 20 mg BID* | | | | FORMULATION | | | |
| | | Mean | SD | CV | 102 20 mg BID* | | | |
| Day | n | (ng/mL) | (ng/mL) | (%) | | Mean | SD | CV |
| 2 | 5 | 1.48 | 0.675 | 45.62 | 5 | 0.784 | 0.606 | 77.25 |
| 3 | 5 | 0.673 | 0.342 | 50.77 | 5 | 0.669 | 0.119 | 17.80 |
| 4 | 5 | 1.04 | 0.737 | 70.90 | 5 | 1.08 | 0.200 | 18.51 |

*Study drugs administered twice daily on Days 1-4 and once on Day 5
Note:
Plasma samples analyzed using a bioanalytical method with a range 0.0500 to 50.0 ng/mL;
concentrations reported in ng/mL to 3 significant figures;
concentrations below limit of quantification set to zero (0.00 ng/mL) in the data summarization

TABLE 55
Day 1 6β-Hydroxyoxymorphone Concentration-Time Data
after Administration of the Control Article Opana ER
20 mg and the Test Article Formulation 102 20 mg

| | Control Article | | | | Test Article | | | |
|---|---|---|---|---|---|---|---|---|
| | Opana ER 20 mg | | | | Formulation | | | |
| Time | | Mean | SD | CV | 102 20 mg | | | |
| (hr) | n | (ng/mL) | (ng/mL) | (%) | | Mean | SD | CV |
| 0.00 | 5 | 0.00 | 0.00 | NC | 5 | 0.00 | 0.00 | NC |
| 0.17 | 5 | 0.00 | 0.00 | NC | 5 | 0.00 | 0.00 | NC |
| 0.33 | 5 | 0.00 | 0.00 | NC | 5 | 0.00 | 0.00 | NC |
| 0.67 | 5 | 0.00 | 0.00 | NC | 5 | 0.00 | 0.00 | NC |
| 1.00 | 5 | 0.00494 | 0.0110 | 223.61 | 5 | 0.00634 | 0.0142 | 223.61 |
| 1.50 | 5 | 0.0482 | 0.0197 | 40.82 | 5 | 0.0189 | 0.0199 | 105.50 |
| 2.00 | 5 | 0.0675 | 0.0438 | 64.85 | 5 | 0.0233 | 0.0225 | 96.65 |
| 3.00 | 5 | 0.0634 | 0.0371 | 58.50 | 5 | 0.0247 | 0.0269 | 108.93 |
| 4.00 | 5 | 0.0504 | 0.0240 | 47.71 | 5 | 0.0306 | 0.0272 | 88.77 |
| 6.00 | 5 | 0.0386 | 0.00940 | 24.35 | 5 | 0.0342 | 0.00969 | 28.30 |
| 8.00 | 5 | 0.0367 | 0.0248 | 67.50 | 5 | 0.0335 | 0.00871 | 25.95 |
| 12.00 | 5 | 0.0665 | 0.0549 | 82.58 | 5 | 0.0273 | 0.0291 | 106.51 |

Note:
Plasma samples analyzed using a bioanalytical method with a range 0.0200 to 20.0 ng/mL;
concentrations reported in ng/mL to 3 significant figures;
concentrations below limit of quantification set to zero (0.00 ng/mL) in the data summarization
NC = Not calculated

TABLE 56
Day 5 6β-Hydroxyoxymorphone Concentration-Time Data
after Administration of the Control Article Opana ER 20
mg and the Test Article Formulation 102 20 mg Twice Daily

| | Control Article | | | | Test Article | | | |
|---|---|---|---|---|---|---|---|---|
| | Opana 20 mg ER BID* | | | | Formulation 102 20 | | | |
| Time | | Mean | SD | CV | mg BID* | | | |
| (hr) | n | (ng/mL) | (ng/mL) | (%) | | Mean | SD | CV |
| 0.00 | 5 | 0.127 | 0.0732 | 57.53 | 5 | 0.0658 | 0.0232 | 35.31 |
| 0.17 | 5 | 0.129 | 0.0631 | 49.07 | 5 | 0.0732 | 0.0222 | 30.28 |
| 0.33 | 5 | 0.131 | 0.0554 | 42.39 | 5 | 0.104 | 0.0451 | 43.46 |
| 0.67 | 5 | 0.128 | 0.0508 | 39.64 | 5 | 0.143 | 0.0715 | 50.14 |
| 1.00 | 5 | 0.141 | 0.0536 | 38.11 | 5 | 0.154 | 0.0700 | 45.46 |
| 1.50 | 5 | 0.158 | 0.0618 | 39.21 | 5 | 0.148 | 0.0686 | 46.44 |
| 2.00 | 5 | 0.171 | 0.0766 | 44.71 | 5 | 0.134 | 0.0549 | 40.90 |
| 3.00 | 5 | 0.173 | 0.0665 | 38.51 | 5 | 0.133 | 0.0478 | 35.88 |
| 4.00 | 5 | 0.151 | 0.0590 | 39.00 | 5 | 0.129 | 0.0409 | 31.65 |
| 6.00 | 5 | 0.125 | 0.0215 | 17.18 | 5 | 0.118 | 0.0432 | 36.71 |
| 8.00 | 5 | 0.121 | 0.0310 | 25.67 | 5 | 0.112 | 0.0349 | 31.08 |
| 12.00 | 5 | 0.198 | 0.0664 | 33.48 | 5 | 0.144 | 0.0724 | 50.34 |
| 16.00 | 5 | 0.170 | 0.0688 | 40.54 | 5 | 0.206 | 0.181 | 88.12 |
| 24.00 | 5 | 0.0876 | 0.0579 | 66.06 | 5 | 0.0609 | 0.0435 | 71.50 |
| 48.00 | 5 | 0.00 | 0.00 | NC | 5 | 0.00 | 0.00 | NC |

*Study drugs administered twice daily on Days 1-4 and once on Day 5
Note:
Plasma samples analyzed using a bioanalytical method with a range 0.0200 to 20.0 ng/mL;
concentrations reported in ng/mL to 3 significant figures;
concentrations below limit of quantification set to zero (0.00 ng/mL) in the data summarization
NC = Not calculated

TABLE 57
Trough 6β-Hydroxyoxymorphone Concentration-Time Data on Days
2, 3, and 4 after Administration of the Control Article Opana
ER 20 mg and the Test Article Formulation 102 20 mg Twice Daily

| | Control Article | | | | Test Article | | | |
|---|---|---|---|---|---|---|---|---|
| | Opana ER 20 mg BID* | | | | Formulation 102 20 | | | |
| | | Mean | SD | CV | mg BID* | | | |
| Day | n | (ng/mL) | (ng/mL) | (%) | | Mean | SD | CV |
| 2 | 5 | 0.104 | 0.0557 | 53.60 | 5 | 0.0523 | 0.0494 | 94.52 |
| 3 | 5 | 0.0668 | 0.0325 | 48.72 | 5 | 0.0493 | 0.0100 | 20.33 |
| 4 | 5 | 0.176 | 0.216 | 122.33 | 5 | 0.0670 | 0.0142 | 21.16 |

*Study drugs administered twice daily on Days 1-4 and once on Day 5
Note:
Plasma samples analyzed using a bioanalytical method with a range 0.0200 to 20.0 ng/mL;
concentrations reported in ng/mL to 3 significant figures;
concentrations below limit of quantification set to zero (0.00 ng/mL) in the data summarization

TABLE 58
Day 1 Oxymorphone-Glucuronide Concentration-Time Data
after Administration of the Control Article Opana ER
20 mg and the Test Article Formulation 102 20 mg

| | Control Article | | | | Test Article | | | |
|---|---|---|---|---|---|---|---|---|
| | Opana ER 20 mg | | | | Formulation | | | |
| Time | | Mean | SD | CV | 102 20 mg | | | |
| (hr) | n | (ng/mL) | (ng/mL) | (%) | | Mean | SD | CV |
| 0.00 | 5 | 0.00 | 0.00 | NC | 5 | 0.00 | 0.00 | NC |
| 0.17 | 5 | 0.00 | 0.00 | NC | 5 | 0.00 | 0.00 | NC |
| 0.33 | 5 | 1.18 | 1.65 | 139.88 | 5 | 0.00 | 0.00 | NC |
| 0.67 | 5 | 55.2 | 51.5 | 93.20 | 5 | 5.64 | 4.61 | 81.62 |
| 1.00 | 5 | 160 | 126 | 78.90 | 5 | 30.7 | 29.9 | 97.62 |
| 1.50 | 5 | 402 | 180 | 44.84 | 5 | 94.7 | 81.4 | 86.02 |
| 2.00 | 5 | 546 | 149 | 27.22 | 5 | 142 | 123 | 86.43 |
| 3.00 | 5 | 506 | 213 | 42.02 | 5 | 225 | 157 | 69.83 |
| 4.00 | 5 | 412 | 184 | 44.58 | 5 | 255 | 211 | 82.85 |
| 6.00 | 5 | 290 | 136 | 47.07 | 5 | 285 | 189 | 66.23 |
| 8.00 | 5 | 205 | 51.3 | 25.03 | 5 | 208 | 104 | 50.05 |
| 12.00 | 5 | 181 | 45.9 | 25.37 | 5 | 85.9 | 40.7 | 47.35 |

Note:
Plasma samples analyzed using a bioanalytical method with a range 1.00 to 1000 ng/mL;
concentrations reported in ng/mL to 3 significant figures;
concentrations below limit of quantification set to zero (0.00 ng/mL) in the data summarization
NC = Not calculated

TABLE 59
Day 5 Oxymorphone-Glucuronide Concentration-Time Data after
Administration of the Control Article Opana ER 20 mg and
the Test Article Formulation 102 20 mg Twice Daily

| | Control Article | | | | Test Article | | | |
|---|---|---|---|---|---|---|---|---|
| | Opana ER 20 mg BID* | | | | Formulation 102 20 | | | |
| Time | | Mean | SD | CV | mg BID* | | | |
| (hr) | n | (ng/mL) | (ng/mL) | (%) | | Mean | SD | CV |
| 0.00 | 5 | 182 | 64.0 | 35.15 | 5 | 133 | 53.2 | 40.00 |
| 0.17 | 5 | 173 | 65.0 | 37.66 | 5 | 131 | 53.2 | 40.47 |
| 0.33 | 5 | 205 | 66.4 | 32.45 | 5 | 295 | 166 | 56.17 |
| 0.67 | 5 | 324 | 191 | 58.97 | 5 | 935 | 628 | 67.20 |
| 1.00 | 5 | 423 | 268 | 63.31 | 5 | 1120 | 669 | 59.71 |
| 1.50 | 5 | 547 | 382 | 69.92 | 5 | 960 | 455 | 47.33 |
| 2.00 | 5 | 641 | 311 | 48.54 | 5 | 725 | 189 | 26.00 |
| 3.00 | 5 | 649 | 236 | 36.35 | 5 | 603 | 139 | 23.12 |
| 4.00 | 5 | 528 | 190 | 36.00 | 5 | 554 | 140 | 25.28 |
| 6.00 | 5 | 320 | 87.0 | 27.21 | 5 | 381 | 39.8 | 10.43 |
| 8.00 | 5 | 297 | 87.6 | 29.50 | 5 | 258 | 37.6 | 14.56 |
| 12.00 | 5 | 255 | 76.1 | 29.83 | 5 | 177 | 18.5 | 10.45 |
| 16.00 | 5 | 176 | 55.5 | 31.52 | 5 | 156 | 72.0 | 46.21 |
| 24.00 | 5 | 69.7 | 38.3 | 54.98 | 5 | 47.3 | 25.7 | 54.36 |
| 48.00 | 5 | 5.64 | 1.71 | 30.32 | 5 | 6.95 | 2.36 | 33.97 |

*Study drugs administered twice daily on Days 1-4 and once on Day 5
Note:
Plasma samples analyzed using a bioanalytical method with a range 1.00 to 1000 ng/mL;
concentrations reported in ng/mL to 3 significant figures;
concentrations below limit of quantification set to zero (0.00 ng/mL) in the data summarization

TABLE 60
Trough Oxymorphone-Glucuronide Concentration-Time Data on Days
2, 3, and 4 after Administration of the Control Article Opana ER
20 mg and the Test Article Formulation 102 20 mg Twice Daily

| | Control Article | | | | Test Article | | | |
|---|---|---|---|---|---|---|---|---|
| | Opana ER 20 mg BID* | | | | Formulation 102 20 | | | |
| | | Mean | SD | CV | mg BID* | | | |
| Day | n | (ng/mL) | (ng/mL) | (%) | | Mean | SD | CV |
| 2 | 5 | 228 | 114 | 50.10 | 5 | 127 | 76.7 | 60.34 |
| 3 | 5 | 149 | 72.2 | 48.28 | 5 | 149 | 59.1 | 39.70 |
| 4 | 5 | 160 | 76.8 | 47.89 | 5 | 184 | 69.0 | 37.44 |

*Study drugs administered twice daily on Days 1-4 and once on Day 5
Note:
Plasma samples analyzed using a bioanalytical method with a range 1.00 to 1000 ng/mL;
concentrations reported in ng/mL to 3 significant figures;
concentrations below limit of quantification set to zero (0.00 ng/mL) in the data summarization

TABLE 61
Day 1 Total Exposure after Administration of the Control Article
Opana ER 20 mg and Test Article Formulation 102 20 mg

| | Control Article | | | | Test Article | | | |
|---|---|---|---|---|---|---|---|---|
| | Opana ER 20 mg | | | | Formulation | | | |
| Time | | Mean | SD | CV | 102 20 mg | | | |
| (hr) | n | (nmol/L) | (nmol/L) | (%) | | Mean | SD | CV |
| 0.00 | 5 | 0.000 | 0.000 | NC | 5 | 0.000 | 0.000 | NC |
| 0.17 | 5 | 0.000 | 0.000 | NC | 5 | 0.03342 | 0.07472 | 223.61 |
| 0.33 | 5 | 3.017 | 3.797 | 125.88 | 5 | 0.3087 | 0.6016 | 194.90 |
| 0.67 | 5 | 119.1 | 109.4 | 91.86 | 5 | 14.11 | 10.15 | 71.89 |
| 1.00 | 5 | 340.5 | 265.1 | 77.88 | 5 | 68.34 | 63.14 | 92.39 |
| 1.50 | 5 | 856.8 | 370.7 | 43.26 | 5 | 204.7 | 171.9 | 83.95 |
| 2.00 | 5 | 1158 | 312.9 | 27.03 | 5 | 303.5 | 258.8 | 85.29 |
| 3.00 | 5 | 1070 | 448.5 | 41.90 | 5 | 476.5 | 333.5 | 69.98 |
| 4.00 | 5 | 869.0 | 386.4 | 44.46 | 5 | 539.6 | 447.9 | 83.00 |
| 6.00 | 5 | 612.4 | 288.0 | 47.02 | 5 | 601.8 | 398.0 | 66.15 |
| 8.00 | 5 | 433.4 | 107.3 | 24.76 | 5 | 438.9 | 218.4 | 49.77 |
| 12.00 | 5 | 383.3 | 97.91 | 25.54 | 5 | 181.9 | 86.20 | 47.39 |

Note:
Plasma concentrations in ng/mL were converted to nmol/L using Molecular Weight and summed across time points to calculate total exposure
NC = Not calculated

TABLE 62
Day 5 Total Exposure after Administration of the Control Article
Opana ER 20 mg and Test Article Formulation 102 20 mg Twice Daily

| | Control Article | | | | Test Article | | | |
|---|---|---|---|---|---|---|---|---|
| | Opana ER 20 mg BID* | | | | Formulation 102 20 | | | |
| Time | | Mean | SD | CV | mg BID* | | | |
| (hr) | n | (nmol/L) | (nmol/L) | (%) | | Mean | SD | CV |
| 0.00 | 5 | 385.6 | 136.2 | 35.33 | 5 | 281.4 | 112.2 | 39.89 |
| 0.17 | 5 | 366.9 | 137.6 | 37.50 | 5 | 289.9 | 115.2 | 39.73 |
| 0.33 | 5 | 436.4 | 143.1 | 32.80 | 5 | 658.4 | 371.4 | 56.42 |
| 0.67 | 5 | 689.7 | 409.5 | 59.38 | 5 | 2001 | 1342 | 67.04 |
| 1.00 | 5 | 899.8 | 573.4 | 63.73 | 5 | 2380 | 1418 | 59.56 |
| 1.50 | 5 | 1164 | 807.6 | 69.39 | 5 | 2035 | 960.2 | 47.17 |
| 2.00 | 5 | 1362 | 658.1 | 48.32 | 5 | 1535 | 395.5 | 25.76 |
| 3.00 | 5 | 1374 | 498.7 | 36.29 | 5 | 1277 | 296.3 | 23.20 |
| 4.00 | 5 | 1116 | 402.5 | 36.06 | 5 | 1172 | 295.5 | 25.22 |
| 6.00 | 5 | 677.7 | 182.5 | 26.93 | 5 | 806.7 | 84.32 | 10.45 |
| 8.00 | 5 | 630.0 | 185.2 | 29.39 | 5 | 547.3 | 79.95 | 14.61 |
| 12.00 | 5 | 543.1 | 160.5 | 29.55 | 5 | 376.9 | 40.04 | 10.62 |
| 16.00 | 5 | 374.3 | 117.1 | 31.27 | 5 | 332.2 | 155.6 | 46.85 |
| 24.00 | 5 | 148.1 | 81.30 | 54.88 | 5 | 100.3 | 54.57 | 54.40 |
| 48.00 | 5 | 11.89 | 3.663 | 30.82 | 5 | 14.70 | 5.023 | 34.18 |

Pharmacokinetic parameters for oxymorphone, 6β-hydroxymorphone, and oxymorphone-glucuronide are summarized in Tables 63-68 below. Relative bioavailability results are shown in Table 69 and 70 and accumulation factors are shown in Table 71.

TABLE 63
Day 1 Pharmacokinetic Parameters of Oxymorphone after Administration of
the Control Article Opana ER 20 mg and Test Article Formulation 102 20 mg

| | Control Article | | | | Test Article | | | |
|---|---|---|---|---|---|---|---|---|
| | Opana ER 20 mg | | | | Formulation | | | |
| Parameter | n | Mean | SD | CV % | 102 20 mg | | | |
| Tₘₐₓ (hr) | 5 | 1.50 | 0.35 | 23.57 | 5 | 3.00 | 1.97 | 65.62 |
| Cₘₐₓ (ng/mL) | 5 | 4.73 | 2.77 | 58.68 | 5 | 2.39 | 1.24 | 51.72 |
| AUC₀₋₁₂ (hr*ng/mL) | 5 | 19.66 | 5.581 | 28.39 | 5 | 13.55 | 7.171 | 52.91 |
| AUCₗₐₛₜ (hr*ng/mL) | 5 | 19.66 | 5.581 | 28.39 | 5 | 13.55 | 7.171 | 52.91 |
| Tₗₐₛₜ (hr) | 5 | 12.00 | 0.00 | 0.00 | 5 | 12.00 | 0.00 | 0.00 |
| Cₗₐₛₜ (ng/mL) | 5 | 1.13 | 0.641 | 56.72 | 5 | 0.525 | 0.291 | 55.38 |

Note:
Full precision data used in pharmacokinetic analysis

TABLE 64
Day 5 Pharmacokinetic Parameters of Oxymorphone after Administration of the Control
Article Opana ER 20 mg and Test Article Formulation 102 20 mg Twice Daily

| | Control Article | | | | Test Article | | | |
|---|---|---|---|---|---|---|---|---|
| | Opana ER 20 mg BID* | | | | Formulation | | | |
| Parameter | n | Mean | SD | CV % | 102 20 mg BID* | | | |
| Tₘₐₓ (hr) | 5 | 2.90 | 2.88 | 99.34 | 5 | 1.00 | 1.13 | 113.09 |
| Cₘₐₓ (ng/mL) | 5 | 6.64 | 3.26 | 49.07 | 5 | 13.7 | 6.58 | 47.94 |
| AUC₀₋₁₂ (hr*ng/mL) | 5 | 32.45 | 7.285 | 22.45 | 5 | 38.11 | 10.95 | 28.74 |
| AUC₀₋₂₄ (hr*ng/mL) | 5 | 47.27 | 10.39 | 21.98 | 5 | 51.15 | 18.93 | 37.02 |
| AUC₀₋₄₈ (hr*ng/mL) | 5 | 53.71 | 11.32 | 21.08 | 5 | 55.42 | 20.76 | 37.47 |
| AUCₗₐₛₜ (hr*ng/mL) | 5 | 51.32 | 12.93 | 25.20 | 5 | 54.55 | 21.14 | 38.76 |
| AUC_{inf} (hr*ng/mL) | 5 | 53.23 | 11.76 | 22.08 | 5 | 55.41 | 20.98 | 37.87 |
| AUC_{Extrap} (%) | 5 | 3.98 | 6.03 | 151.27 | 5 | 1.77 | 1.00 | 56.10 |
| λ_{z} (hr⁻¹) | 5 | 0.1516 | 0.0705 | 46.53 | 5 | 0.1275 | 0.0548 | 42.97 |
| T_{1/2} (hr) | 5 | 5.39 | 2.22 | 41.16 | 5 | 6.33 | 2.72 | 43.01 |
| Tₗₐₛₜ (hr) | 5 | 33.60 | 13.15 | 39.12 | 5 | 38.40 | 13.15 | 34.23 |
| Cₗₐₛₜ (ng/mL) | 5 | 0.220 | 0.233 | 105.96 | 5 | 0.111 | 0.0720 | 64.64 |

*Study drugs administered twice daily on Days 1-4 and once on Day 5
Note:
Full precision data used in pharmacokinetic analysis

TABLE 65
Day 1 Pharmacokinetic Parameters of 6β-Hydroxyoxymorphone after Administration
of the Control Article Opana ER 20 mg and Test Article Formulation 102 20 mg

| | Control Article | | | | Test Article | | | |
|---|---|---|---|---|---|---|---|---|
| | Opana ER 20 mg | | | | Formulation | | | |
| Parameter | n | Mean | SD | CV % | 102 20 mg | | | |
| Tₘₐₓ (hr) | 5 | 4.40 | 4.28 | 97.22 | 5 | 7.20 | 4.60 | 63.95 |
| Cₘₐₓ (ng/mL) | 5 | 0.0919 | 0.0436 | 47.41 | 5 | 0.0524 | 0.0170 | 32.47 |
| AUC₀₋₁₂ (hr*ng/mL) | 5 | 0.5361 | 0.2461 | 45.90 | 5 | 0.3238 | 0.1049 | 32.39 |
| AUCₗₐₛₜ (hr*ng/mL) | 5 | 0.5309 | 0.2544 | 47.92 | 5 | 0.2966 | 0.1123 | 37.86 |
| Tₗₐₛₜ (hr) | 5 | 10.80 | 2.68 | 24.85 | 5 | 10.40 | 2.19 | 21.07 |
| Cₗₐₛₜ (ng/mL) | 5 | 0.0716 | 0.0479 | 66.84 | 5 | 0.0409 | 0.0162 | 39.67 |

Note:
Full precision data used in pharmacokinetic analysis

TABLE 66
Day 5 Pharmacokinetic Parameters of 6β-Hydroxyoxymorphone
after Administration of the Control Article Opana ER 20
mg and Test Article Formulation 102 20 mg Twice Daily

| | Control Article | | | | Test Article | | | |
|---|---|---|---|---|---|---|---|---|
| | Opana ER 20 mg BID* | | | | Formulation | | | |
| Parameter | n | Mean | SD | CV % | 102 20 mg BID* | | | |
| Tₘₐₓ (hr) | 5 | 9.20 | 7.69 | 83.63 | 5 | 6.70 | 6.85 | 102.27 |
| Cₘₐₓ (ng/mL) | 5 | 0.257 | 0.0263 | 10.23 | 5 | 0.239 | 0.164 | 68.68 |
| AUC₀₋₁₂ (hr*ng/mL) | 5 | 1.783 | 0.1821 | 10.21 | 5 | 1.517 | 0.5217 | 34.39 |
| AUC₀₋₂₄ (hr*ng/mL) | 5 | 3.549 | 0.3648 | 10.28 | 5 | 3.282 | 1.787 | 54.45 |
| AUC₀₋₄₈ (hr*ng/mL) | 5 | 4.601 | 0.9807 | 21.32 | 5 | 4.012 | 2.247 | 56.01 |
| AUCₗₐₛₜ | 5 | 3.549 | 0.3648 | 10.28 | 5 | 3.282 | 1.787 | 54.45 |
| Tₗₐₛₜ (hr) | 5 | 24.00 | 0.00 | 0.00 | 5 | 24.00 | 0.00 | 0.00 |
| Cₗₐₛₜ (ng/mL) | 5 | 0.0876 | 0.0579 | 66.06 | 5 | 0.0609 | 0.0435 | 71.50 |

*Study drugs administered twice daily on Days 1-4 and once on Day 5
Note:
Full precision data used in pharmacokinetic analysis

TABLE 67
Day 1 Pharmacokinetic Parameters of Oxymorphone-Glucuronide
after Administration of the Control Article Opana ER
20 mg and Test Article Formulation 102 20 mg

| | Control Article | | | | Test Article | | | |
|---|---|---|---|---|---|---|---|---|
| | Opana ER 20 mg | | | | Formulation | | | |
| Parameter | n | Mean | SD | CV % | 102 20 mg | | | |
| Tₘₐₓ (hr) | 5 | 2.10 | 0.55 | 26.08 | 5 | 3.40 | 1.67 | 49.22 |
| Cₘₐₓ (ng/mL) | 5 | 592 | 164 | 27.67 | 5 | 334 | 190 | 56.81 |
| AUC₀₋₁₂ (hr*ng/mL) | 5 | 3376 | 747.1 | 22.13 | 5 | 2140 | 999.6 | 46.71 |
| AUCₗₐₛₜ (hr*ng/mL) | 5 | 3376 | 747.1 | 22.13 | 5 | 2140 | 999.6 | 46.71 |
| Tₗₐₛₜ (hr) | 5 | 12.00 | 0.00 | 0.00 | 5 | 12.00 | 0.00 | 0.00 |
| Cₗₐₛₜ (ng/mL) | 5 | 181 | 45.9 | 25.37 | 5 | 85.9 | 40.7 | 47.35 |

Note:
Full precision data used in pharmacokinetic analysis

TABLE 68
Day 5 Pharmacokinetic Parameters of Oxymorphone-Glucuronide
after Administration of the Control Article Opana ER 20
mg and Test Article Formulation 102 20 mg Twice Daily

| | Control Article | | | | Test Article | | | |
|---|---|---|---|---|---|---|---|---|
| | Opana ER 20 mg BID* | | | | Formulation | | | |
| Parameter | n | Mean | SD | CV % | 102 20 mg BID* | | | |
| Tₘₐₓ (hr) | 5 | 3.50 | 2.60 | 74.23 | 5 | 1.50 | 0.87 | 57.74 |
| Cₘₐₓ (ng/mL) | 5 | 756 | 299 | 39.52 | 5 | 1270 | 427 | 33.62 |
| AUC₀₋₁₂ (hr*ng/mL) | 5 | 4616 | 1160 | 25.13 | 5 | 5236 | 731.6 | 13.97 |
| AUC₀₋₂₄ (hr*ng/mL) | 5 | 6462 | 1503 | 23.26 | 5 | 6714 | 1089 | 16.23 |
| AUC₀₋₄₈ (hr*ng/mL) | 5 | 7366 | 1953 | 26.51 | 5 | 7364 | 1307 | 17.75 |
| AUCₗₐₛₜ (hr*ng/mL) | 5 | 7366 | 1953 | 26.51 | 5 | 7364 | 1307 | 17.75 |
| Tₗₐₛₜ (hr) | 5 | 48.00 | 0.00 | 0.00 | 5 | 48.00 | 0.00 | 0.00 |
| Cₗₐₛₜ (ng/mL) | 5 | 5.64 | 1.71 | 30.32 | 5 | 6.95 | 2.36 | 33.97 |

*Study drugs administered twice daily on Days 1-4 and once on Day 5
Note:
Full precision data used in pharmacokinetic analysis

TABLE 69
Day 1 Relative Bioavailability of Oxymorphone, Oxymorphone-
Glucuronide, and 6β-Hydroxyoxymorphone after Administration
of the Control Article Opana ER 20 mg and Test Article
Formulation 102 20 mg

| | | Mean | | Relative |
|---|---|---|---|---|
| | | (hr * ng/mL) | | Bioavailability |
| | | Test | Control | (%) |
| Analyte | Parameter | Formulation 102 | | (Test/Control) |
| Oxymorphone | AUC₀₋₁₂ | 13.55 | 19.66 | 68.92 |
| Oxymorphone- | AUC₀₋₁₂ | 2140 | 3376 | 63.39 |
| Glucuronide | | | | |
| 6β-Hydroxyoxy- | AUC₀₋₁₂ | 0.3238 | 0.5361 | 60.40 |
| morphone | | | | |

TABLE 70
Day 5 Relative Bioavailability of Oxymorphone, Oxymorphone-
Glucuronide, and 6β-Hydroxyoxymorphone after Administration
of the Control Article Opana ER 20 mg and Test Article
Formulation 102 20 mg Twice Daily

| | | Mean | | Relative |
|---|---|---|---|---|
| | | (hr * ng/mL) | | Bioavailability |
| | | Test | Control | (%) |
| Analyte | Parameter | Formulation 102 | | (Test/Control) |
| Oxymorphone | AUC₀₋₁₂ | 38.11 | 32.45 | 117.44 |
| Oxymorphone- | AUC₀₋₁₂ | 5236 | 4616 | 113.43 |
| Glucuronide | | | | |
| 6β-Hydroxyoxy- | AUC₀₋₁₂ | 1.517 | 1.783 | 85.08 |
| morphone | | | | |
| Oxymorphone | AUC₀₋₄₈ | 55.42 | 53.71 | 103.18 |
| Oxymorphone- | AUC₀₋₄₈ | 7364 | 7366 | 99.97 |
| Glucuronide | | | | |
| 6β-Hydroxyoxy- | AUC₀₋₄₈ | 4.012 | 4.601 | 87.20 |
| morphone | | | | |

Note:
Study drugs administered twice daily on Days 1-4 and once on Day 5

TABLE 71
Accumulation of Oxymorphone, Oxymorphone-Glucuronide, and 6β-
Hydroxyoxymorphone during Administration of the Control Article
Opana ER 20 mg and Test Article Formulation 102 20 mg Twice Daily

| | | Mean AUC₀₋₁₂ | | |
|---|---|---|---|---|
| | | (hr * ng/mL) | | Accumulation |
| Treatment | Analyte | Day 1 | Day 5 | (Day 5/Day 1) |
| Formulation 102 | Oxymorphone | 13.55 | 38.11 | 2.81 |
| Formulation 102 | Oxymorphone-Glucuronide | 2140 | 5236 | 2.45 |
| Formulation 102 | 6β-Hydroxyoxymorphone | 0.3238 | 1.517 | 4.68 |
| Opana ER | Oxymorphone | 19.66 | 32.45 | 1.65 |
| Opana ER | Oxymorphone-Glucuronide | 3376 | 4616 | 1.37 |
| Opana ER | 6β-Hydroxyoxymorphone | 0.5361 | 1.783 | 3.33 |

Attainment of Steady State:

As evidenced by the above tables, due to the lack of continuing increases in predose oxymorphone, 6β-hydroxyoxymorphone, and oxymorphone-glucuronide concentrations on successive dosing days, steady-state conditions appear to have been achieved during the five day, twice-daily dosing regimens of Formulation 102 and Opana ER.

Extent of Exposure:

Exposure to oxymorphone constituted only a small fraction of the total drug exposure, combined data for oxymorphone and its two major metabolites, 6β-hydroxyoxymorphone and oxymorphone-glucuronide. Exposure to these analytes had the following rank order: 6β-hydroxyoxymorphone <oxymorphone <<oxymorphone-glucuronide. In general, concentrations of 6β-hydroxyoxymorphone were approximately 50-fold lower and concentrations of oxymorphone-glucuronide were approximately 100-fold greater than those of the parent drug oxymorphone.

Formulation 102:

Mean estimates of oxymorphone Cmax after Formulation 102 during the first dosing interval on Day 1 and following the last dose on Day 5 were 2.39 ng/mL at 3.00 hr and 13.7 ng/mL at 1.00 hr, respectively. Mean estimates of oxymorphone AUC₀₋₁₂ after Formulation 102 on Day 1 and Day 5 were 13.55 hr*ng/mL and 38.11 hr*ng/mL, respectively. Mean T_{1/2} of oxymorphone after Formulation 102, determined during the extended sampling interval on Day 5, was 6.33 hr. The approximate 6-fold increase in oxymorphone Cₘₐₓ at steady state on Day 5 was unexpected and exceeds the degree of accumulation that would be predicted for a twice-daily regimen of a drug product with a half-life of 6 hours (theoretical accumulation factor 1.33) as well as the actual accumulation determined using AUC₀₋₁₂ values (observed accumulation factor 2.81).

On Day 5, the time at which oxymorphone Cₘₐₓ was observed, Tₘₐₓ, was highly variable and ranged from 0.33 to 3.00 hr for individual animals (113.09% CV). As would be expected, shorter Tₘₐₓ values were associated with higher Cₘₐₓ. The animal with the longest Tₘₐₓ of 3.00 hr had a the lowest Cₘₐₓ at 5.68 ng/mL, a value close to that which would be predicted, based on observed accumulation during multiple dosing. The factors that might have caused the unexpected increase in the early release rate for some animals on Day 5 after administration of Formulation 102 are unknown. However, following the initial absorption and subsequent distribution phase, plasma concentrations of oxymorphone remained relatively constant between approximately 8 and 16 hours post-dose.

Opana ER:

Mean estimates of oxymorphone Cₘₐₓ after Opana ER during the first dosing interval on Day 1 and following the last dose on Day 5 were 4.73 ng/mL at 1.50 hr and 6.64 ng/mL at 2.90 hr, respectively. Mean estimates of oxymorphone AUC₀₋₁₂ after Opana ER on Day 1 and Day 5 were 19.66 hr*ng/mL and 32.45 hr*ng/mL, respectively. Mean T_{1/2} of oxymorphone after Opana ER, determined during the extended sampling interval on Day 5, was 5.39 hr. The actual accumulation during twice-daily administration of Opana ER, determined using AUC₀₋₁₂ values, was 1.65.

Relative Bioavailability:

Based on AUC₀₋₁₂ on Day 1, the bioavailability of oxymorphone after Formulation 102 relative to Opana ER was 68.92%; analogous percent ratios for oxymorphone-glucuronide and 6β-hydroxyoxymorphone were 63.39% and 60.40%, respectively. The relative bioavailability estimates on Day 5 were higher than those on Day 1. Based on Day 5 data, the bioavailability of oxymorphone after Formulation 102 relative to Opana ER ranged from 103.18% (AUC₀₋₄₈) to 117.44% (AUC₀₋₁₂). The (Formulation 102/Opana ER) percent ratios for oxymorphone-glucuronide and 6β-hydroxyoxymorphone ranged from 99.97% (AUC₀₋₄₈) to 113.43% (AUC₀₋₁₂) and 85.08% (AUC₀₋₁₂) to 87.20% (AUC₀₋₄₈), respectively.

In addition to the above pharmacokinetic results, the test article, Formulation 102, was generally well tolerated following repeat oral administration in male beagle dogs and no adverse findings were noted regarding mortality, clinical pathology, and macroscopic observations. The animals that received the test article displayed similar clinical observations, decreases in food consumption, and decreases in body weights as did the animals that received the positive control article, Opana ER.

### Example 16

### Capsule Shell Interaction Study

The formulations indicated in Table 72 were prepared and filled into either hard gelatin or HPMC capsules to evaluate the effect of capsule choice on dissolution and storage time dependent change in mean release of active agent. The Formulation 103 placebo was prepared at 1 kg scale using an overhead mixer. A Sucrose Acetate Isobutyrate (SAIB)/Triacetin(TA)=1.5 stock solution was prepared prior to the compounding process, and the temperature of the process was maintained at 60° C.±5° C. throughout. SAIB/TA (1.50) stock solution was added to a glass jar, and placed into the water bath. Isopropyl Myristate (IPM) was added, and mixed at 600 rpm. Colliodal silicon dioxide (Cab-O-Sil) was added to the solution mixed for 20 minutes. The mixture was homogenized using Fisher PowerGen 500 with a setting of 9600 rpm for 5 minutes. Sieved cellulose acetate butyrate (CAB) was added to the jar while mixing at 1000 rpm followed by 1430 rpm for 35 minutes. Finally, sieved hydroxyethyl cellulose (HEC) was added into the jar and mixed for 30 minutes to complete the formulation. The active formulation was prepared in 250 g scale. For Formulation 103, approximately 13 grams of oxycodone base was weighed out and mixed with 240 gram of placebo formulation in a separate bottle until uniform. The Formulation 104 placebo was prepared similarly to the above with the exception of adding Gelucire 44/14 in the formulation. For Formulation 104, approximately 27 grams of oxycodone base was weighed out and mixed with 236 gram of placebo formulation in a separate bottle until uniform.

Placebo and active formulations were manually filled into white opaque hard gelatin capsules (Capsugel Licap size 0) with filling weight of 585 mg. The same fill weight was filled into white HPMC capsules (Qualicaps Quali-V size 0). For Formulation 103, 30 mg capsules were made. For Formulation 104, 60 mg capsules were made.

TABLE 72

| | Formulation | Formulation |
|---|---|---|
| | 103 | 104 |
| | 30 mg | 60 mg |
| Component | % Weight | % Weight |
| Oxycodone Base | 5.13 | 10.26 |
| SAIB | 40.98 | 35.87 |
| Triacetin | 27.32 | 26.57 |
| IPM | 14.23 | 14.36 |
| CAB | 4.74 | 4.94 |
| HEC | 5.69 | 4.49 |
| Cab-O-Sil | 1.90 | 2.15 |
| BHT | 0.02 | 0.02 |
| Gelucire 44/14 | — | 1.35 |

In addition, the water content of the empty capsules was determined by Karl Fischer titration generally as set forth in USP <921> Method 1C using an AquaStar C3000 Karl Fischer Coulometric Titrator. The results of the Karl Fischer titration showing the difference in water content between the empty gelatin and HPMC capsules are provided below in Table 73.

TABLE 73

| Sample | W. (mg) | Ave. | Stdev | % CV |
|---|---|---|---|---|
| Licaps ® Gelatin | 94.44 | 15.28 | 0.02 | 0.1 |
| Capules | 95.30 | 14.86 | 0.07 | 0.5 |
| | 97.91 | 15.44 | 0.11 | 0.7 |
| | Grand = | 15.19 | 0.30 | 2.0 |
| Quali-V ® HPMC | 92.30 | 4.72 | 0.03 | 0.6 |
| Capules | 96.60 | 4.72 | 0.15 | 3.2 |
| | Grand = | 4.72 | 0.00 | 0.1 |

The release rate of oxycodone base was determined from six capsules using a USP Apparatus 2 dissolution tester. Dissolution medium containing 1000 ml 0.1 N HCl with 0.5% (w/w) SDS was maintained at 37° C. with 100 rpm paddle speed over the course of the 24 hour dissolution test. A 20 mesh screen hanging basket was incorporated to hold the test article. The standard sampling time points were 0.5, 2, 3, 6, 12, 18 and 24 hours. A 1 mL sample was taken at each time point and assayed using reverse-phase HPLC at 240 nm wavelength. The mobile phase included 0.35% (w/v) SDS/0.7% (v/v) acetic acid/44% (v/v) acetonitrile in water.

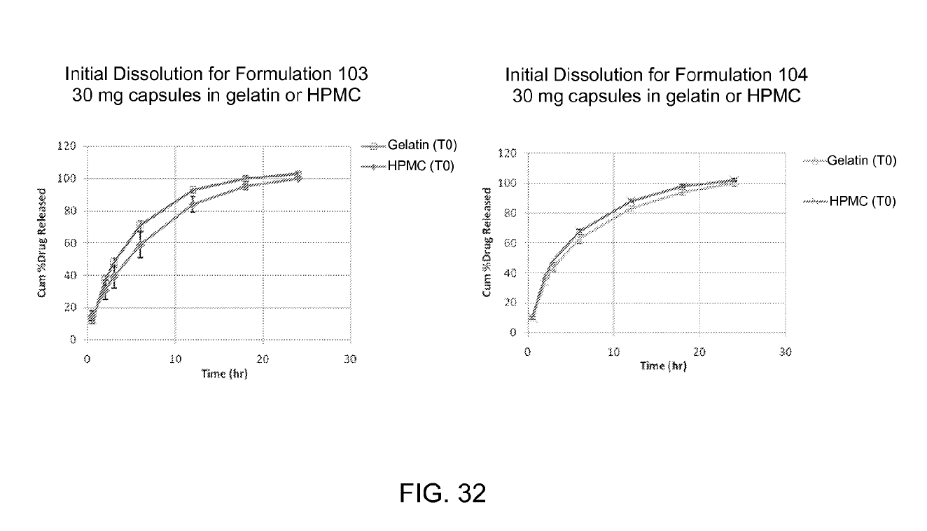

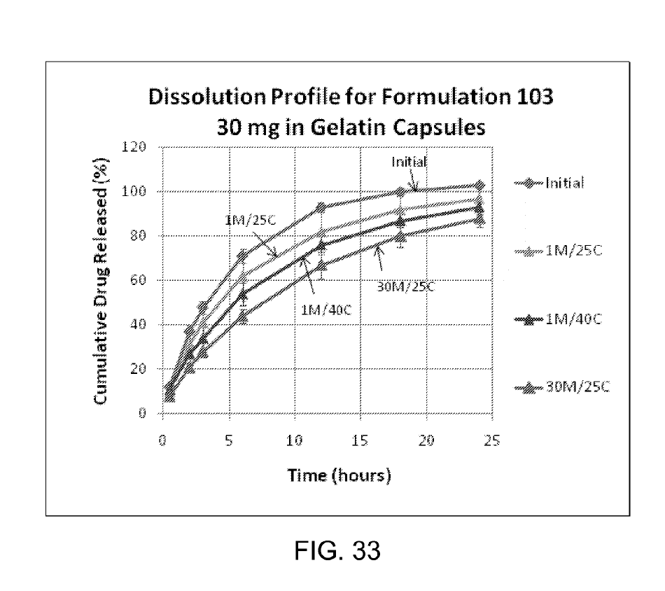

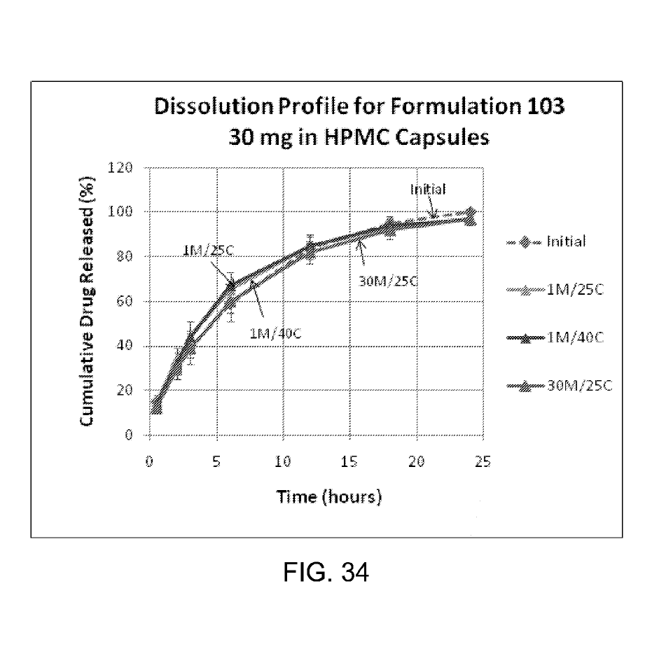

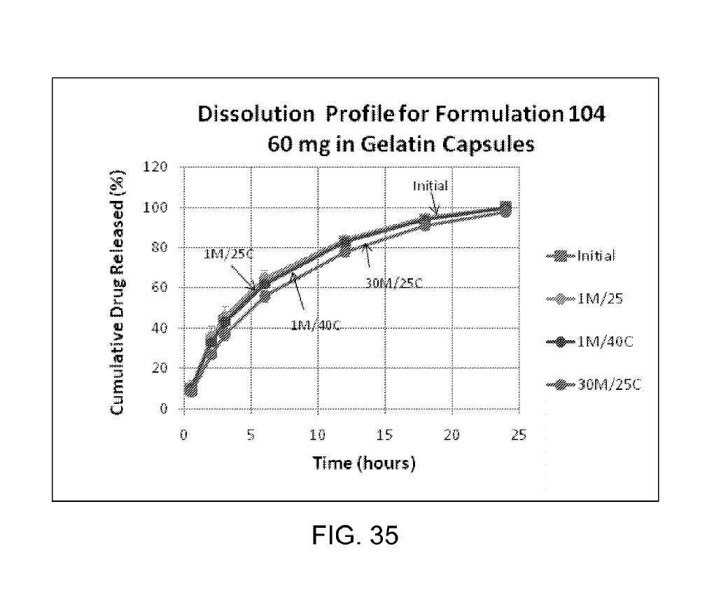

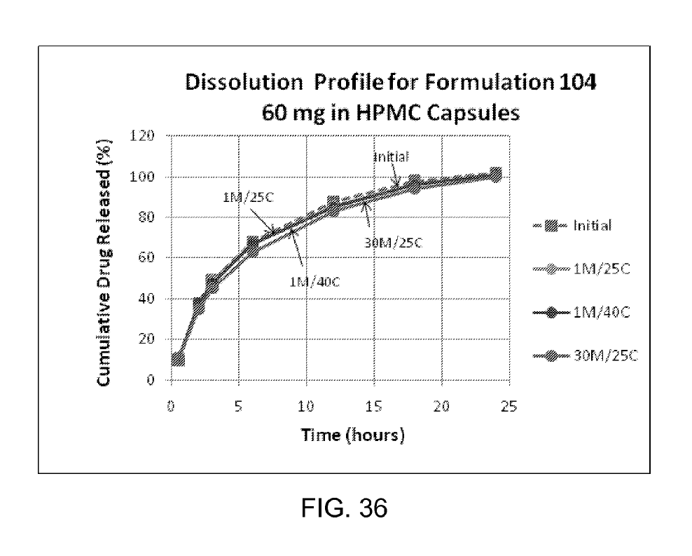

The initial dissolution results at T=0 for Formulations 103 and 104 in gelatin and HPMC capsules are provided in FIG. 32. FIG. 33 shows a storage time-dependent change in mean release of the active agent for Formulation 103 in gelatin capsules when stored for 1 month at 25° C. and 40° C., or for 30 months at 25° C. As shown in FIG. 34, Formulation 103 exhibited greater stability in HPMC capsules as evidenced by a decrease in the storage time-dependent change in mean release of the active agent. FIG. 35 shows dissolution results for Formulation 104 in gelatin capsules when stored for 1 month at 25° C. and 40° C., or for 30 months at 25° C. FIG. 36 shows dissolution results for Formulation 104 in HPMC capsules when stored for 1 month at 25° C. and 40° C., or for 30 months at 25° C. Formulation 104 exhibited good stability in both gelatin and HPMC capsules as evidenced by the absence of a significant storage time-dependent change in mean release of the active agent. Without intending to be bound by any particular theory, it appears that Formulation 103 filled in hard gelatin capsules showed dissolution changes due to potential interaction between the capsule and the formulation. Formulation 104 showed good product stability without dissolution change in both types of capsule shell.

What is claimed is:

1. A composition comprising:
an opioid;
about 15% by weight to about 45% by weight, based on total weight of the composition, of a solvent;
about 1% by weight to about 20% by weight, based on total weight of the composition, of a rheology modifier;
a mineral particle; and
about 1% to about 2.5% by weight, based on total weight of the composition, of water.

2. The composition of claim 1, wherein the opioid is a mu opioid agonist.

3. The composition of any one of claims 1 and 2, wherein the opioid is selected from oxycodone, oxymorphone, hydrocodone, and hydromorphone, either in the free base form or a pharmaceutically acceptable salt form thereof.

4. The composition of any one of claims 1 to 3, wherein the opioid is oxycodone.

5. The composition of any one of claims 1 to 4, wherein the opioid is present in the composition at about 2% by weight to about 50% by weight relative to the total weight of the composition.

6. The composition of any one of claims 1 to 5, wherein the opioid is present in the composition at about 0.1% by weight to about 20% by weight relative to the total weight of the composition.

7. The composition of any one of claims 1 to 6, wherein the solvent is a hydrophilic solvent.

8. The composition of any one of claims 1 to 7, wherein the solvent is triacetin.

9. The composition of any one of claims 1 to 7, wherein the solvent is ethyl lactate.

10. The composition of any one of claims 1 to 9, wherein the rheology modifier is isopropyl myristate (IPM).

11. The composition of any one of claims 1 to 10, wherein the mineral particle comprises silicon dioxide, carnauba wax, or cetyl alcohol.

12. The composition of any one of claims 1 to 11, wherein the mineral particle comprises silicon dioxide.

13. The composition of any one of claims 1 to 12, wherein the mineral particle is present in the composition in an amount from about 1.9% by weight to about 3.0% by weight relative to the total weight of the composition.

14. The composition of any one of claims 1 to 13, further comprising a high viscosity liquid carrier material (HVLCM) having a viscosity of at least 5000 cP at 37° C. that does not crystallize neat at 25° C. and 1 atmosphere.

15. The composition of claim 14, wherein the HVLCM is sucrose acetate isobutyrate (SAIB).

16. The composition of any one of claims 1 to 15, further comprising a network former.

17. The composition of claim 16, wherein the network former comprises cellulose acetate butyrate (CAB).

18. The composition of any one of claims 16 and 17, wherein the composition comprises about 4% by weight to about 5% by weight of the network former relative to the total weight of the composition.

19. The composition of any one of claims 1 to 18, wherein the composition is contained within a capsule.

20. The composition of any one of claims 1 to 19, wherein the composition is contained within a capsule having a water content of less than about 10% by weight.

21. The composition of any one of claims 1 to 20, wherein the composition is within a hydroxypropylmethylcellulose (HPMC) capsule.

22. A composition as defined in any one of claims 1 to 21, for use as a medicament.

23. A composition as defined in any one of claims 1 to 21, for use in a method of treating pain.

24. Use of a composition as defined in any one of claims 1 to 21 for the manufacture of a medicament for treating pain.

25. A method for treating pain in a subject, the method comprising orally administering to the subject a composition as defined in any one of claims 1 to 21, wherein one or more symptoms or signs associated with the subject's pain is alleviated.
